(19) United States
(12) Patent Application Publication
(10) Pub. No.: US 2023/0062391 A1
(43) Pub. Date: Mar. 2, 2023

(54) NUCLEIC ACID MOLECULES COMPRISING CLEAVABLE OR EXCISABLE MOIETIES

(71) Applicant: ULTIMA GENOMICS, INC., Newark, CA (US)

(72) Inventors: Daniel MAZUR, San Diego, CA (US); Florian OBERSTRASS, Menlo Park, CA (US)

(21) Appl. No.: 17/814,780

(22) Filed: Jul. 25, 2022

Related U.S. Application Data
(63) Continuation of application No. PCT/US2021/016081, filed on Feb. 1, 2021.
(60) Provisional application No. 62/969,100, filed on Feb. 2, 2020.

(30) Foreign Application Priority Data
Feb. 10, 2020 (US) PCT/US2020/017491
Nov. 5, 2020 (IB) PCT/IB2020/060420

(51) Int. Cl. C12Q 1/6806; C12Q 1/6853; C12Q 1/6874

(52) U.S. Cl. CPC C12Q 1/6806; C12Q 1/6853; C12Q 1/6874

(57) ABSTRACT

The present disclosure provides compositions comprising nucleic acid molecules coupled to supports and comprising one or more cleavable or excisable moieties. Methods of enriching and sequencing nucleic acid molecules are also provided.

### CROSS REFERENCE TO RELATED APPLICATIONS

This application claims the benefit of priority of International Patent Applications No. PCT/US2020/017491 filed Feb. 10, 2020 and PCT/IB2020/060420 filed Nov. 5, 2020, and U.S. Provisional Patent Application No. 62/969,100, filed Feb. 2, 2020, the contents of which are all incorporated herein by reference in their entirety.

### FIELD OF INVENTION

The present invention is in the field of nucleic acid processing.

### BACKGROUND

With growing interest in patient-specific therapeutic modalities, and the constant ongoing research at the genome level, the ability to rapidly, accurately and cheaply sequence the entire genome both for large and small applications is highly sought. However, technical difficulties still exist that must be overcome. Sequencing is often performed on beads with clonal populations of single-stranded DNA; however, due to the numerous repeats present in the human genome these single-stranded molecules often cross-hybridize, leading to bead clumping, dropping out of material, and loss of full genome coverage. Further, single-stranded molecules inherently fold on themselves, creating secondary structure that can both impede progression of sequencing and generate context bias in base calling. The ability to sequence from double-stranded clonal populations, and indeed to carry out all preparation and processing steps with double-stranded molecules, is thus greatly desired.

### SUMMARY

The present invention provides compositions comprising double-stranded nucleic acid molecules linked to solid supports and comprising cleavable bases. Methods of isolating and/or enriching for complexes comprising double-stranded nucleic acid molecules, comprising excising cleavable bases within the molecules, whether as a pre-enrichment step before or as a post-enrichment step after clonal amplification, are provided. Methods of sequencing double-stranded nucleic acid molecules, comprising excising cleavable bases within the molecules, both in combination and without the enrichment described herein, are also provided.

According to a first aspect, there is provided a method of analyzing a nucleic acid molecule, the method comprising:
a) providing the nucleic acid molecule, wherein the nucleic acid molecule comprises (i) a first strand comprising at least two cleavable or excisable moieties and (ii) a second strand, and wherein the nucleic acid molecule is coupled to a support;
b) subjecting the nucleic acid molecule coupled to the support to conditions sufficient to cleave or excise one or more of the at least two cleavable or excisable moieties, thereby generating a cleaved nucleic acid molecule coupled to the support, wherein the cleaved nucleic acid molecule comprises a nick or gap region;
c) bringing the cleaved nucleic acid molecule into contact with a polymerase enzyme and a labeled nucleotide, wherein the labeled nucleotide is configured to emit a signal;
d) subjecting the cleaved nucleic acid molecule to conditions sufficient to incorporate the labeled nucleotide into the nick or gap region of the cleaved nucleic acid molecule or into a newly synthesized strand complementary to the second strand; and
e) detecting a signal or change in signal from the cleaved nucleic acid molecule, wherein the signal or the change in signal is indicative of incorporation of the labeled nucleotide into the nick or gap region of the cleaved nucleic acid molecule or into the newly synthesized strand complementary to the second strand;
   thereby analyzing the nucleic acid molecule.

According to some embodiments, (b) comprises contacting the nucleic acid molecule with a cleaving agent configured to cleave or excise the one or more of the at least two cleavable or excisable moieties.

According to some embodiments, the cleaving agent is selected from the group consisting of uracil DNA glycosylase (UDG), apyrimidinic/apurinic endonuclease (APE), endonucleases (e.g., endonuclease VIII (EndoVIII) or V (EndoV)), uracil-specific excision reagent (USER) enzyme, formamidopyrimidine DNA glycosylase (Fpg), 8-oxoguanine glycosylase (OGG1), RNase (e.g., RNaseH, such as RNaseHII), ultraviolet light, and a combination thereof.

According to some embodiments, the at least two cleavable or excisable moieties are independently selected from the group consisting of a ribonucleic acid (RNA) base, a uracil base, an inosine base, 2,6-diamino-4-hydroxy-5-formamidopyrimidine (FapyG) base, 8-oxo-7,8-dihydroguanine (8oxoG) base, and a photocleavable base.

According to some embodiments, two or more of the at least two cleavable or excisable moieties are of different types.

According to some embodiments, the nick or gap region comprises a gap region of two or more bases in the first strand of the nucleic acid molecule.

According to some embodiments, the nick or gap region comprises a target nucleic acid sequence, or a complement thereof.

According to some embodiments, the nick or gap region is adjacent to a target nucleic acid sequence, or a complementary nucleic acid sequence thereof.

According to some embodiments, the labeled nucleotide is not terminated.

According to some embodiments, the labeled nucleotide comprises a fluorescent dye.

According to some embodiments, the nick or gap region comprises a free 3′ end capable of priming a polymerizing reaction by the polymerase enzyme.

According to some embodiments, (c) comprises bringing the cleaved nucleic acid molecule into contact with a solution comprising a plurality of nucleotides comprising the labeled nucleotide, wherein each nucleotide of the plurality of nucleotides is of a same type.

According to some embodiments, each nucleotide of the plurality of nucleotides is a labeled nucleotide.

According to some embodiments, an additional nucleotide of the plurality of nucleotides is incorporated into the nick or gap region or into the newly synthesized strand complementary to the second strand.

According to some embodiments, the nucleic acid molecule comprises a deoxyribonucleic acid (DNA).

According to some embodiments, the nucleic acid molecule comprises a ribonucleic acid (RNA).

According to some embodiments, the support is a particle.

According to some embodiments, the particle is immobilized to a surface.

According to some embodiments, prior to (b), the nucleic acid molecule further comprises or is coupled to a capture entity.

According to some embodiments, the capture entity comprises one or more members selected from the group consisting of biotin, avidin, a nucleic acid sequence, a charged particle, and a magnetic particle.

According to some embodiments, the capture entity is configured to couple to a capturing entity.

According to some embodiments, the capturing entity comprises one or more members selected from the group consisting of biotin, avidin, a nucleic acid sequence, an electric field system, and a magnetic field system.

According to some embodiments, the capture entity is coupled to the capturing entity.

According to some embodiments, the capturing entity is coupled to an additional support.

According to some embodiments, the additional support is a particle.

According to some embodiments, the capture entity is proximal to a free end of the nucleic acid molecule and wherein at least one cleavable or excisable moiety is proximal to the capture entity such that excision of the cleavable or excisable moiety dissociates the capture entity from the nucleic acid molecule.

According to some embodiments, the subjecting in (b) decouples the nucleic acid molecule from the additional support, or wherein the method further comprises before step (b) subjecting the nucleic acid molecule coupled to the support to conditions sufficient to cleave or excise the at least one cleavable or excisable moiety proximal to the capture entity.

According to some embodiments, the polymerase enzyme is a strand-displacement polymerase enzyme.

According to some embodiments, prior to (c), the nucleic acid molecule is situated within a partition.

According to some embodiments, prior to (b), the nucleic acid molecule is situated within the partition and step (b) is performed within the partition.

According to some embodiments, (c) is performed outside of the partition.

According to some embodiments, the partition is a droplet of an emulsion.

According to some embodiments, the partition is a well.

According to some embodiments, the method further comprises repeating (c) and (d) with an additional nucleotide, wherein the labeled nucleotide and the additional nucleotide are of different types, optionally wherein different types are different canonical nucleobases.

According to some embodiments, subjecting the cleaved nucleic acid molecule to conditions sufficient to incorporate the additional nucleotide in (d) comprises separating a portion of the first strand and a portion of the second strand.

According to some embodiments, the additional nucleotide is a labeled nucleotide.

According to some embodiments, the method further comprises repeating (e).

According to some embodiments, the method is a method of analyzing a plurality of nucleic acid molecules and wherein the plurality of nucleic acid molecules are coupled to the support.

According to some embodiments, the plurality of nucleic acid molecules comprises a common nucleic acid sequence.

According to some embodiments, the common nucleic acid sequence is a target nucleic acid sequence.

According to some embodiments, the plurality of nucleic acid molecules comprises a clonal population of nucleic acid molecules.

According to some embodiments, the method is a method of pair-end analysis, the second strand comprises at least one additional cleavable or excisable moiety and subsequent to step (e) the method comprises:
f) subjecting the cleaved nucleic acid molecule coupled to the support to conditions sufficient to cleave or excise one or more of the at least one additional cleavable or excisable moiety, thereby generating a twice-cleaved nucleic acid molecule coupled to the support, wherein the twice-cleaved nucleic acid molecule comprises a nick or gap region in the second strand;
g) bringing the twice-cleaved nucleic acid molecule into contact with a polymerase enzyme and a labeled nucleotide, wherein the labeled nucleotide is configured to emit a signal;
h) subjecting the cleaved nucleic acid molecule to conditions sufficient to incorporate the labeled nucleotide into the nick or gap region of the second strand of the twice-cleaved nucleic acid molecule or into a newly synthesized strand complementary to the first strand; and
i) detecting a signal or change in signal from the twice-cleaved nucleic acid molecule, wherein the signal or the change in signal is indicative of incorporation of the labeled nucleotide into the nick or gap region of the second strand of the twice-cleaved nucleic acid molecule or into the newly synthesized strand complementary to the first strand; thereby pair-end analyzing the nucleic acid molecule.

According to some embodiments, the method comprises before step (f) integrating an additional nucleotide than is a terminated nucleotide that does not allow 3′ polymerization.

According to some embodiments, the additional cleavable or excisable moiety of the second strand is cleaved by a different condition than the at least two cleavable or excisable moieties of the first strand.

According to some embodiments, the polymerase enzyme of step (g) is a strand-displacing polymerase enzyme.

According to some embodiments, the nick or gap region on the second strand comprises a gap region of two or more bases.

According to some embodiments, the nick or gap region on the second strand is adjacent to a second target nucleic acid sequence, or a complement thereof.

According to some embodiments, the second strand is directly coupled to the support and the first strand is not directly coupled to the support.

According to some embodiments, the method further comprises repeating (g) and (h) with an additional nucleotide, wherein the labeled nucleotide and the additional nucleotide are of different types, optionally wherein different types are different canonical nucleobases.

According to some embodiments, the additional nucleotide is a labeled nucleotide.

According to some embodiments, the method further comprises repeating (e).

According to another aspect, there is provided a method of analyzing a nucleic acid molecule, the method comprising:
a) providing the nucleic acid molecule, wherein the nucleic acid molecule comprises (i) a first strand comprising at least a first cleavable or excisable moieties and (ii) a second strand comprising at least a second cleavable or excisable moiety, and wherein the nucleic acid molecule is coupled to a support;
b) subjecting the nucleic acid molecule coupled to the support to conditions sufficient to cleave or excise the at least a first cleavable or excisable moiety, thereby generating a cleaved nucleic acid molecule coupled to the support, wherein the cleaved nucleic acid molecule comprises a nick or gap region in the first strand;
c) bringing the cleaved nucleic acid molecule into contact with a polymerase enzyme and a labeled nucleotide, wherein the labeled nucleotide is configured to emit a signal;
d) subjecting the cleaved nucleic acid molecule to conditions sufficient to incorporate the labeled nucleotide into the nick or gap region of the first strand or into a newly synthesized strand complementary to the second strand;
e) detecting a signal or change in signal from the cleaved nucleic acid molecule, wherein the signal or the change in signal is indicative of incorporation of the labeled nucleotide into the nick or gap region of the first strand or into the newly synthesized strand complementary to the second strand;
f) subjecting the nucleic acid molecule coupled to the support or the cleaved nucleic acid molecule to conditions sufficient to cleave or excise the at least a second cleavable or excisable moiety, thereby generating a twice-cleaved nucleic acid molecule coupled to the support, wherein the twice-cleaved nucleic acid molecule comprises a nick or gap region in the second strand;
g) bringing the twice-cleaved nucleic acid molecule into contact with a polymerase enzyme and a labeled nucleotide, wherein the labeled nucleotide is configured to emit a signal;
h) subjecting the twice-cleaved nucleic acid molecule to conditions sufficient to incorporate the labeled nucleotide into the nick or gap region of the second strand or into a newly synthesized strand complementary to the first strand; and
i) detecting a signal or change in signal from the twice-cleaved nucleic acid molecule, wherein the signal or the change in signal is indicative of incorporation of the labeled nucleotide into the nick or gap region of the second strand or into the newly synthesized strand complementary to the first strand;
   thereby analyzing a nucleic acid molecule.

According to some embodiments, step (b) and step (f) are performed simultaneously.

According to some embodiments, step (c) and step (g) are performed simultaneously, step (d) and step (h) are performed simultaneously, and step (e) and step (i) are performed simultaneously.

According to some embodiments, (b), (f) or both comprise contacting the nucleic acid molecule with a cleaving agent configured to cleave or excise the at least first cleavable or excisable moiety, the at least second cleavable or excisable moiety or both.

According to some embodiments, the cleaving agent is selected from the group consisting of uracil DNA glycosylase (UDG), apyrimidinic/apurinic endonuclease (APE), endonucleases (e.g., endonuclease VIII (EndoVIII) or V (EndoV)), uracil-specific excision reagent (USER) enzyme, formamidopyrimidine DNA glycosylase (Fpg), 8-oxoguanine glycosylase (OGG1), RNase (e.g., RNaseH, such as RNaseHII), ultraviolet light, and a combination thereof.

According to some embodiments, the at least first and second cleavable or excisable moieties are independently selected from the group consisting of a ribonucleic acid (RNA) base, a uracil base, an inosine base, 2,6-diamino-4-hydroxy-5-formamidopyrimidine (FapyG) base, 8-oxo-7,8-dihydroguanine (8oxoG) base, and a photocleavable base.

According to some embodiments, the at least a first cleavable or excisable moiety and the at least a second cleavable or excisable moiety are of different types.

According to some embodiments, the at least a first cleavable or excisable moiety and the at least a second cleavable or excisable moiety are of the same type.

According to some embodiments, the nick or gap region of the first strand, the second strand, or both comprise a gap region of two or more bases.

According to some embodiments, the nick or gap region of the first strand, the second strand, or both comprise a target nucleic acid sequence, or a complement thereof.

According to some embodiments, the nick or gap region of the first strand, the second strand, or both are adjacent to a target nucleic acid sequence, or a complement thereof.

According to some embodiments, the labeled nucleotide is not terminated.

According to some embodiments, the labeled nucleotide comprises a fluorescent dye.

According to some embodiments, the nick or gap region of the first strand, the second strand, or both each comprise a free 3′ end capable of priming a polymerizing reaction by the polymerase enzyme.

According to some embodiments, (c), (g) or both comprise bringing the cleaved nucleic acid molecule into contact with a solution comprising a plurality of nucleotides comprising the labeled nucleotide, wherein each nucleotide of the plurality of nucleotides is of a same type.

According to some embodiments, each nucleotide of the plurality of nucleotides is a labeled nucleotide.

According to some embodiments, an additional nucleotide of the plurality of nucleotides is incorporated into the nick or gap region of the first strand, the second strand or both or into the newly synthesized strand complementary to the second strand, the newly synthesized strand complementary to the first strand or both.

According to some embodiments, the nucleic acid molecule comprises a deoxyribonucleic acid (DNA).

According to some embodiments, the nucleic acid molecule comprises a ribonucleic acid (RNA).

According to some embodiments, the support is a particle.

According to some embodiments, the particle is immobilized to a surface.

According to some embodiments, prior to (b), the nucleic acid molecule further comprises or is coupled to a capture entity.

According to some embodiments, the capture entity comprises one or more members selected from the group consisting of biotin, avidin, a nucleic acid sequence, a charged particle, and a magnetic particle.

According to some embodiments, the capture entity is configured to couple to a capturing entity.

According to some embodiments, the capturing entity comprises one or more members selected from the group consisting of biotin, avidin, a nucleic acid sequence, an electric field system, and a magnetic field system.

According to some embodiments, the capture entity is coupled to the capturing entity.

According to some embodiments, the capturing entity is coupled to an additional support.

According to some embodiments, the additional support is a particle.

According to some embodiments, the capture entity is proximal to a free end of the nucleic acid molecule and wherein at least one additional cleavable or excisable moiety is proximal to the capture entity such that excision of the additional cleavable or excisable moiety dissociates the capture entity from the nucleic acid molecule.

According to some embodiments, the subjecting in (b) decouples the nucleic acid molecule from the additional support, or wherein the method further comprises before step (b) subjecting the nucleic acid molecule coupled to the support to conditions sufficient to cleave or excise the at least one additional cleavable or excisable moiety proximal to the capture entity.

According to some embodiments, the polymerase enzyme is a strand-displacement polymerase enzyme.

According to some embodiments, prior to (c), the nucleic acid molecule is situated within a partition.

According to some embodiments, prior to (b), the nucleic acid molecule is situated within the partition and step (b) is performed within the partition.

According to some embodiments, (c) is performed outside of the partition.

According to some embodiments, the partition is a droplet of an emulsion.

According to some embodiments, the partition is a well.

According to some embodiments, the method further comprises repeating (c) and (d), (g) and (h), or both with an additional nucleotide, wherein the labeled nucleotide and the additional nucleotide are of different types, optionally wherein different types are different canonical nucleobases.

According to some embodiments, subjecting the cleaved nucleic acid molecule to conditions sufficient to incorporate the additional nucleotide in (d), (h) or both comprises separating a portion of the first strand and a portion of the second strand.

According to some embodiments, the additional nucleotide is a labeled nucleotide.

According to some embodiments, the method further comprises repeating (e), (i) or both.

According to some embodiments, the method is a method of analyzing a plurality of nucleic acid molecules and wherein the plurality of nucleic acid molecules are coupled to the support.

According to some embodiments, the plurality of nucleic acid molecules comprises a common nucleic acid sequence.

According to some embodiments, the common nucleic acid sequence comprises a first and a second target nucleic acid sequence.

According to some embodiments, the plurality of nucleic acid molecules comprises a clonal population of nucleic acid molecules.

According to some embodiments, the second strand is directly coupled to the support and the first strand is not directly coupled to the support.

According to another aspect, there is provided a method of analyzing a nucleic acid molecule, the method comprising:
a) providing the nucleic acid molecule, wherein the nucleic acid molecule comprises (i) a first strand and (ii) a second strand comprising at least one cleavable or excisable moiety, and wherein the nucleic acid molecule is coupled to a support by a 5′ end of the second strand;
b) subjecting the nucleic acid molecule coupled to the support to conditions sufficient to cleave or excise the at least one cleavable or excisable moiety, thereby generating a cleaved nucleic acid molecule coupled to the support, wherein the cleaved nucleic acid molecule comprises a nick or gap region in the second strand;
c) bringing the cleaved nucleic acid molecule into contact with a polymerase enzyme and a labeled nucleotide, wherein the labeled nucleotide is configured to emit a signal;
d) subjecting the cleaved nucleic acid molecule to conditions sufficient to incorporate the labeled nucleotide into the nick or gap region of the second strand or into a newly synthesized strand complementary to the first strand, wherein the labeled nucleotide is incorporated into a nucleic acid polymer directly coupled to the support;
e) detecting a signal or change in signal from the cleaved nucleic acid molecule, wherein the signal or the change in signal is indicative of incorporation of the labeled nucleotide into the nick or gap region of the second strand or into the newly synthesized strand complementary to the first strand;
f) repeating (c), (d) and (e) with additional nucleotides, wherein the labeled nucleotide and the additional nucleotide are of different types,
g) dissociating the first strand from the second strand, thereby generating the support coupled to the second strand and not the first strand;
h) bringing the support coupled to the second strand and not the first strand into contact with a polymerase enzyme, a labeled nucleotide, and an oligonucleotide primer complementary to a 3′ region of the second strand, wherein the labeled nucleotide is configured to emit a signal;
i) subjecting the support coupled to the second strand and not the first strand to conditions sufficient to incorporate the labeled nucleotide into a newly synthetizing first strand 3′ to the oligonucleotide primer and complementary to the second strand; and
j) detecting a signal or change in signal from the support coupled to the second strand and oligonucleotide primer, wherein the signal or the change in signal is indicative of incorporation of the labeled nucleotide into the newly synthesizing first strand complementary to the second strand; thereby analyzing a nucleic acid molecule.

According to some embodiments. (b) comprises contacting the nucleic acid molecule with a cleaving agent configured to cleave or excise the at least one cleavable or excisable moiety.

According to some embodiments, the cleaving agent is selected from the group consisting of uracil DNA glycosylase (UDG), apyrimidinic/apurinic endonuclease (APE), endonucleases (e.g., endonuclease VIII (EndoVIII) or V (EndoV)), uracil-specific excision reagent (USER) enzyme, formamidopyrimidine DNA glycosylase (Fpg), 8-oxoguanine glycosylase (OGG1), RNase (e.g., RNaseH, such as RNaseHII), ultraviolet light, and a combination thereof.

According to some embodiments, the at least one cleavable or excisable moiety is selected from the group consisting of a ribonucleic acid (RNA) base, a uracil base, an inosine base, 2,6-diamino-4-hydroxy-5-formamidopyrimidine (FapyG) base, 8-oxo-7,8-dihydroguanine (8oxoG) base, and a photocleavable base.

According to some embodiments, the nick or gap region of the second strand comprises a gap region of two or more bases.

According to some embodiments, the polymerase enzyme is a strand-displacing polymerase enzyme.

According to some embodiments, the method further comprises before step (c) bringing the cleaved nucleic acid molecule into contact with an exonuclease enzyme and subjecting the cleaved nucleic acid molecule to conditions sufficient to degrade at least a portion of the second strand 3′ to the nick or gap region.

According to some embodiments, the nick or gap region is a nick of only a single base.

According to some embodiments, the polymerase enzyme is not a strand displacing polymerase enzyme.

According to some embodiments, the exonuclease enzyme is a 5′ to 3′ exonuclease enzyme.

According to some embodiments, the nick or gap region comprises a target nucleic acid sequence, or a complement thereof.

According to some embodiments, the first strand comprises a target nucleic acid sequence, or a complement thereof.

According to some embodiments, the nick or gap region is adjacent to a target nucleic acid sequence, or a complement thereof.

According to some embodiments, the labeled nucleotide is not terminated.

According to some embodiments, the labeled nucleotide comprises a fluorescent dye.

According to some embodiments, the nick or gap region comprises a free 3′ end capable of priming a polymerizing reaction by the polymerase enzyme.

According to some embodiments, (c), (h) or both comprise bringing the cleaved nucleic acid molecule into contact with a solution comprising a plurality of nucleotides comprising the labeled nucleotide, wherein each nucleotide of the plurality of nucleotides is of a same type.

According to some embodiments, each nucleotide of the plurality of nucleotides is a labeled nucleotide.

According to some embodiments, an additional nucleotide of the plurality of nucleotides is incorporated into the nick or gap region of the second strand, or into the newly synthesized strand complementary to the second strand, the newly synthesized strand complementary to the first strand or both.

According to some embodiments, the nucleic acid molecule comprises a deoxyribonucleic acid (DNA).

According to some embodiments, the nucleic acid molecule comprises a ribonucleic acid (RNA).

According to some embodiments, the support is a particle.

According to some embodiments, the particle is immobilized to a surface.

According to some embodiments, prior to (b), the nucleic acid molecule further comprises or is coupled to a capture entity.

According to some embodiments, the capture entity comprises one or more members selected from the group consisting of biotin, avidin, a nucleic acid sequence, a charged particle, and a magnetic particle.

According to some embodiments, the capture entity is configured to couple to a capturing entity.

According to some embodiments, the capturing entity comprises one or more members selected from the group consisting of biotin, avidin, a nucleic acid sequence, an electric field system, and a magnetic field system.

According to some embodiments, the capture entity is coupled to the capturing entity.

According to some embodiments, the capturing entity is coupled to an additional support.

According to some embodiments, the additional support is a particle.

According to some embodiments, the capture entity is proximal to a free end of the nucleic acid molecule and wherein at least one additional cleavable or excisable moiety is proximal to the capture entity such that excision of the additional cleavable or excisable moiety dissociates the capture entity from the nucleic acid molecule.

According to some embodiments, the subjecting in (b) decouples the nucleic acid molecule from the additional support, or wherein the method further comprises before step (b) subjecting the nucleic acid molecule coupled to the support to conditions sufficient to cleave or excise the at least one additional cleavable or excisable moiety proximal to the capture entity.

According to some embodiments, prior to (c), the nucleic acid molecule is situated within a partition.

According to some embodiments, prior to (b), the nucleic acid molecule is situated within the partition and step (b) is performed within the partition.

According to some embodiments, (c) is performed outside of the partition.

According to some embodiments, the partition is a droplet of an emulsion.

According to some embodiments, the partition is a well.

According to some embodiments, the method further comprises repeating (h) and (i), or both with an additional nucleotide, wherein the labeled nucleotide and the additional nucleotide are of different types, optionally wherein different types are different canonical nucleobases.

According to some embodiments, the additional nucleotide is a labeled nucleotide.

According to some embodiments, the method further comprises repeating (j).

According to some embodiments, the method is a method of analyzing a plurality of nucleic acid molecules and wherein the plurality of nucleic acid molecules are coupled to the support.

According to some embodiments, the plurality of nucleic acid molecules comprises a common nucleic acid sequence.

According to some embodiments, the common nucleic acid sequence comprises a first and a second target nucleic acid sequence.

According to some embodiments, the plurality of nucleic acid molecules comprises a clonal population of nucleic acid molecules.

According to another aspect, there is provided a composition comprising a nucleic acid molecule coupled to a support, wherein the nucleic acid molecule comprises a first strand and a second strand, and wherein the first strand (i) comprises at least three cleavable or excisable moieties and (ii) comprises or is coupled to a capture entity.

According to some embodiments, the second strand of the nucleic acid molecule is devoid of cleavable or excisable moieties.

According to some embodiments, the second strand of the nucleic acid molecule is coupled to the support.

According to some embodiments, the first strand of the nucleic acid molecule is coupled to the support.

According to some embodiments, a cleavable or excisable moiety of the at least three cleavable or excisable moieties is selected from a ribonucleic acid (RNA) base, a uracil base, an inosine base, 2,6-diamino-4-hydroxy-5-formamidopyrimidine (FapyG) base, 8-oxo-7,8-dihydroguanine (8oxoG) base, and a photocleavable base.

According to some embodiments, the nucleic acid molecule comprises deoxyribonucleic acid (DNA) and the at least three cleavable or excisable moieties are RNA bases, and wherein the nucleic acid molecule is devoid of RNA bases other than the at least three cleavable or excisable moieties.

According to some embodiments, the first strand of the nucleic acid molecule comprises a target nucleic acid sequence flanked by a first adapter and a second adapter, wherein the first adapter is disposed at a first end of the first strand and the second adapter is disposed at a second end of the first strand, and wherein the first adapter comprises the at least three cleavable or excisable moieties.

According to some embodiments, the second strand comprises a third adapter comprising a sequence complementary to a sequence of the first adapter and a fourth adapter comprising a sequence complementary to a sequence of the second adapter, wherein the third adapter is disposed at a first end of the second strand and the fourth adapter is disposed at a second end of the second strand.

According to some embodiments, the second strand is coupled to the support via the fourth adapter.

According to some embodiments, a first cleavable or excisable moiety of the at least three cleavable or excisable moieties is proximal to an end of the first strand of the nucleic acid molecule.

According to some embodiments, the end is a 5′ end.

According to some embodiments, the end is a 3′ end.

According to some embodiments, the first cleavable or excisable moiety is within 3 bases of the end of the first strand of the nucleic acid molecule.

According to some embodiments, the first cleavable or excisable moiety and a second cleavable or excisable moiety of the at least three cleavable or excisable moieties are separated by at least ten bases.

According to some embodiments, the first cleavable or excisable moiety and a second cleavable or excisable moiety of the at least three cleavable or excisable moieties are separated by fewer than ten bases.

According to some embodiments, cleavage or excision of the first cleavable or excisable moiety and a second cleavable or excisable moiety is configured to induce dissociation of one or more bases from the second strand of the nucleic acid molecule.

According to some embodiments, cleavage or excision of the first cleavable or excisable moiety and a second cleavable or excisable moiety is configured to not induce dissociation of one or more bases from the second strand of the nucleic acid molecule.

According to some embodiments, the first cleavable or excisable moiety and a third cleavable or excisable moiety of the at least three cleavable or excisable moieties are separated by at least ten bases.

According to some embodiments, the second cleavable or excisable moiety and the third cleavable or excisable moiety are separated by at least ten bases.

According to some embodiments, the second cleavable or excisable moiety and the third cleavable or excisable moiety are separated by fewer than ten bases.

According to some embodiments, the first cleavable or excisable moiety and a third cleavable or excisable moiety of the at least three cleavable or excisable moieties are separated by fewer than ten bases.

According to some embodiments, cleavage or excision of any one cleavable or excisable moiety of the at least three cleavable or excisable moieties is configured to not induce dissociation of one or more bases from the second strand of the nucleic acid molecule.

According to some embodiments, the capture entity comprises one or more members selected from the group consisting of biotin, avidin, a nucleic acid sequence, a charged particle, and a magnetic particle.

According to some embodiments, the capture entity comprises biotin.

According to some embodiments, the capture entity comprises a nucleic acid sequence.

According to some embodiments, the capture entity comprises a magnetic particle or a charged particle.

According to some embodiments, the composition further comprises an additional support comprising a capturing entity, wherein the capture entity and the capturing entity are configured to couple to one another.

According to some embodiments, the additional support is a particle.

According to some embodiments, the additional support is or comprises a magnetic moiety.

According to some embodiments, the capturing entity comprises one or more members selected from the group consisting of biotin, avidin, a nucleic acid sequence, an electric field system, and a magnetic field system.

According to some embodiments, the support is a particle.

According to some embodiments, the support is or comprises a magnetic moiety.

According to some embodiments, the nucleic acid molecule is derived from a cell.

According to some embodiments, the nucleic acid molecule is included within a partition.

According to some embodiments, the partition is a droplet of an emulsion.

According to some embodiments, the partition is a well.

According to some embodiments, the nucleic acid molecule comprises a deoxyribonucleic acid (DNA).

According to some embodiments, the nucleic acid molecule comprises a ribonucleic acid (RNA).

According to another aspect, there is provided a composition comprising a nucleic acid molecule coupled to a support, wherein the nucleic acid molecule comprises a first strand and a second strand, and wherein the first strand comprises at least a first cleavable or excisable moiety proximal to a free 5′ end of the first strand and (ii) the second strand comprises at least a second cleavable or excisable moiety proximal to a 5′ end coupled to the support.

According to some embodiments, the first cleavable or excisable moiety, the second cleavable or excisable moiety, or both is selected from a ribonucleic acid (RNA) base, a uracil base, an inosine base, 2,6-diamino-4-hydroxy-5-formamidopyrimidine (FapyG) base, 8-oxo-7,8-dihydroguanine (8oxoG) base, and a photocleavable base.

According to some embodiments, the first and the second cleavable or excisable moieties are cleaved in the same conditions.

According to some embodiments, the first and the second cleavable or excisable moieties are cleaved in different conditions.

According to some embodiments, the nucleic acid molecule comprises deoxyribonucleic acid (DNA) and at least one of the first and second cleavable or excisable moieties are RNA bases, and wherein the nucleic acid molecule is devoid of RNA bases other than the first and second cleavable or excisable moieties.

According to some embodiments, the first strand of the nucleic acid molecule comprises a target nucleic acid sequence flanked by a first adapter and a second adapter, wherein the first adapter is disposed at a first end of the first strand and the second adapter is disposed at a second end of the first strand, and wherein the first adapter comprises the at least a first cleavable or excisable moiety.

According to some embodiments, the second strand comprises a third adapter comprising a sequence complementary to a sequence of the first adapter and a fourth adapter comprising a sequence complementary to a sequence of the second adapter, wherein the third adapter is disposed at a first end of the second strand and the fourth adapter is disposed at a second end of the second strand, and wherein the four adapter comprises the at least a second cleavable or excisable moiety.

According to some embodiments, the at least a first cleavable or excisable moiety is within 30 bases of the free 5′ end of the first strand of the nucleic acid molecule, the at least a second cleavable or excisable moiety is within 30 bases of the 5′ end coupled to the support of the second strand of the nucleic acid molecule.

According to some embodiments, the at least a first cleavable or excisable moiety and a complementary base to the at least a second cleavable or excisable moiety are separated by at least 100 bases.

According to some embodiments, the at least a first cleavable or excisable moiety is a plurality of first cleavable or excisable moieties and cleavage or excision of the first cleavable or excisable moieties is configured to induce dissociation of one or more intervening bases from the second strand of the nucleic acid molecule.

According to some embodiments, the at least a first cleavable or excisable moiety is a plurality of first cleavable or excisable moieties and the first cleavable or excisable moieties are separated by less than ten bases.

According to some embodiments, the at least a second cleavable or excisable moiety is a plurality of second cleavable or excisable moieties and cleavage or excision of the second cleavable or excisable moieties is configured to induce dissociation of one or more intervening bases from the first strand of the nucleic acid molecule.

According to some embodiments, the at least a second cleavable or excisable moiety is a plurality of second cleavable or excisable moieties and the second cleavable or excisable moieties are separated by less than ten bases.

According to some embodiments, the at least a first cleavable or excisable moiety is a plurality of first cleavable or excisable moieties and cleavage or excision of the first cleavable or excisable moieties is configured to not induce dissociation of one or more intervening bases from the second strand of the nucleic acid molecule.

According to some embodiments, the at least a first cleavable or excisable moiety is a plurality of first cleavable or excisable moieties and the first cleavable or excisable moieties are separated by at least ten bases.

According to some embodiments, the nucleic acid molecule further comprises or is coupled to a capture entity.

According to some embodiments, the capture entity comprises one or more members selected from the group consisting of biotin, avidin, a nucleic acid sequence, a charged particle, and a magnetic particle.

According to some embodiments, the capture entity is configured to couple to a capturing entity.

According to some embodiments, the capturing entity comprises one or more members selected from the group consisting of biotin, avidin, a nucleic acid sequence, an electric field system, and a magnetic field system.

According to some embodiments, the capture entity is coupled to the capturing entity.

According to some embodiments, the capturing entity is coupled to an additional support.

According to some embodiments, the additional support is a particle.

According to some embodiments, the capture entity is proximal to a free end of the nucleic acid molecule and wherein at least one additional cleavable or excisable moiety is proximal to the capture entity, such that excision of the additional cleavable or excisable moiety dissociates the capture entity from the nucleic acid molecule.

According to some embodiments, the additional cleavable or excisable moiety is cleaved or excised in the same conditions as the first cleavable or excisable moiety, in the same condition as the second cleavable or excisable moiety or both.

According to some embodiments, the first strand comprises the capture entity proximal to a free 5′ end of the first strand.

According to some embodiments, the additional cleavable or excisable moiety is a base comprising the capture entity or is 3′ to a base comprising the capture entity and cleavage or excision of the additional cleavable or excisable moiety dissociates the base comprising the capture entity from the second strand.

According to some embodiments, the additional cleavable or excisable moiety is less than 10 nucleotides from the free 5′ end of the first strand.

According to some embodiments, the at least a first cleavable or excisable moiety and the additional cleavable or excisable moiety are sufficiently separated such that cleavage of the at least a first cleavable or excisable moiety and the additional cleavable or excisable moiety does not dissociate intervening bases from the second strand.

According to some embodiments, the at least a first cleavable or excisable moiety and the additional cleavable or excisable moiety are separated by at least 10 bases.

According to some embodiments, the nucleic acid molecule is derived from a cell.

According to some embodiments, the nucleic acid molecule is included within a partition.

According to some embodiments, the partition is a droplet of an emulsion.

According to some embodiments, the partition is a well.

According to some embodiments, the nucleic acid molecule comprises a deoxyribonucleic acid (DNA).

According to some embodiments, the nucleic acid molecule comprises a ribonucleic acid (RNA).

According to another aspect, there is provided a method of processing a nucleic acid molecule, the method comprising:
a) providing a solution comprising a nucleic acid molecule coupled to a support, wherein the nucleic acid molecule comprises a first strand and a second strand, wherein (i) the first strand comprises a cleavable or excisable moiety proximal to an end of the first strand and (ii) the first strand comprises or is coupled to a capture entity at or proximal to the end;
b) bringing the solution into contact with a capturing entity under conditions sufficient to couple the capture entity and the capturing entity;
c) separating the nucleic acid molecule coupled to the capturing entity from other components of the solution; and
d) subsequent to (c), subjecting the nucleic acid molecule coupled to the capturing entity to conditions sufficient to cleave or excise the cleavable or excisable moiety, thereby uncoupling the nucleic acid molecule and the capturing entity; thereby processing the nucleic acid molecule.

According to some embodiments, the method further comprises, prior to (a):
(i) providing the support coupled to a single-stranded nucleic acid molecule, wherein the single-stranded nucleic acid molecule comprises the second strand of the nucleic acid molecule, or a portion thereof;
(ii) providing a primer molecule comprising a nucleic acid sequence that is complementary to a nucleic acid sequence at or near an end of the single-stranded nucleic acid molecule distal to the support, wherein the primer molecule comprises the cleavable or excisable moiety and comprises or is coupled to the capture entity; and
(iii) subjecting the single-stranded nucleic acid molecule and the primer molecule to conditions sufficient to hybridize the primer molecule to the single-stranded nucleic acid molecule.

According to some embodiments, the method further comprises, subsequent to (iii), (iv) subjecting the primer molecule hybridized to the single-stranded nucleic acid molecule to conditions sufficient to extend the primer molecule to generate the first strand of the nucleic acid molecule, or a portion thereof.

According to some embodiments, the method further comprises repeating (i)-(iv) for a plurality of single-stranded nucleic acid molecules coupled to the support.

According to some embodiments, the primer molecule is coupled to a particle.

According to some embodiments, the primer molecule is releasably coupled to the particle.

According to some embodiments, the method further comprises subjecting the primer molecule coupled to the particle to conditions sufficient to release the primer molecule from the particle.

According to some embodiments, the method further comprises, prior to (a):
(i) providing the support coupled to a primer molecule comprising a first nucleic acid sequence;
(ii) providing a template nucleic acid molecule comprising a second nucleic acid sequence that is complementary to the first nucleic acid sequence of the primer molecule, w % herein the template nucleic acid molecule comprises a non-cleavable or excisable moiety that is complementary to the cleavable or excisable moiety of the nucleic acid molecule; and
(iii) subjecting the template nucleic acid molecule and the primer molecule to conditions sufficient to hybridize the template nucleic acid molecule to the primer molecule.

According to some embodiments, the method further comprises, subsequent to (iii), (iv) subjecting the primer molecule hybridized to the single-stranded nucleic acid molecule to conditions sufficient to extend the primer molecule to generate the second strand of the nucleic acid molecule, or a portion thereof.

According to some embodiments, the method further comprises (v) subjecting the second strand of the nucleic acid molecule hybridized to the template nucleic acid molecule to conditions sufficient to separate the template nucleic acid molecule and the second strand.

According to some embodiments, the template nucleic acid molecule is coupled to a particle.

According to some embodiments, the template nucleic acid molecule is releasably coupled to the particle.

According to some embodiments, the method further comprises subjecting the template nucleic acid molecule coupled to the particle to conditions sufficient to release the template nucleic acid molecule from the particle.

According to some embodiments, the method further comprises repeating (i)-(v) for a plurality of primer molecules coupled to the support, thereby generating a plurality of second strands coupled to the support.

According to some embodiments, the method further comprises (vi) providing an additional primer molecule comprising (A) a third nucleic acid sequence that is complementary to a fourth nucleic acid sequence of the second strand and (B) the cleavable or excisable moiety, or non-cleavable or excisable analog thereof, wherein the additional primer molecule comprises or is coupled to the capture entity; (vii) subjecting the second strand and the additional primer molecule to conditions sufficient to hybridize the additional primer molecule to the second strand; and (viii) subjecting the additional primer molecule hybridized to the second strand to conditions sufficient to extend the additional primer molecule to generate the first strand of the nucleic acid molecule, or a portion thereof.

According to some embodiments, the additional primer molecule is coupled to a particle.

According to some embodiments, the additional primer molecule is releasably coupled to the particle.

According to some embodiments, the method further comprises subjecting the additional primer molecule coupled to the particle to conditions sufficient to release the additional primer molecule from the particle.

According to some embodiments, the method further comprises repeating (vi)-(viii) for a plurality of additional primer molecules, thereby generating a plurality of first strands.

According to some embodiments, the solution comprises a plurality of supports coupled to a plurality of nucleic acid molecules comprising a plurality of capture entities and a plurality of cleavable or excisable moieties, wherein each nucleic acid molecule of the plurality of nucleic acid molecules comprises a first strand and a second strand, wherein (i) each the first strand comprises a cleavable or excisable moiety of the plurality of cleavable or excisable moieties proximal to a free end of each the first strand and (ii) each the first strand comprises or is coupled to a capture entity of the plurality of capture entities at or proximal to the free end.

According to some embodiments, the solution further comprises a plurality of additional supports, which plurality of additional supports are not coupled to nucleic acid molecules comprising a plurality of capture entities.

According to some embodiments, prior to (b), the support is included within a partition.

According to some embodiments, the nucleic acid molecule is coupled to the support within a partition.

According to some embodiments, the partition is a droplet of an emulsion.

According to some embodiments, the partition is a well.

According to some embodiments, the cleavable or excisable moiety is selected from the group consisting of a ribonucleic acid (RNA) base, a uracil base, an inosine base, 2,6-diamino-4-hydroxy-5-formamidopyrimidine (FapyG) base, 8-oxo-7,8-dihydroguanine (8oxoG) base, and a photocleavable base.

According to some embodiments, the nucleic acid molecule comprises deoxyribonucleic acid (DNA) and the cleavable or excisable moiety is an RNA base, and wherein the nucleic acid molecule is devoid of RNA bases other than the cleavable or excisable moiety.

According to some embodiments, the conditions in (d) comprise bringing the nucleic acid molecule in contact with a cleaving agent configured to cleave or excise the cleavable or excisable moiety.

According to some embodiments, the cleaving agent is selected from the group consisting of uracil DNA glycosylase (UDG), apyrimidinic/apurinic endonuclease (APE), endonucleases (e.g., endonuclease VIII (EndoVIII) or V (EndoV)), uracil-specific excision reagent (USER) enzyme, formamidopyrimidine DNA glycosylase (Fpg), 8-oxoguanine glycosylase (OGG1), RNase (e.g., RNaseH, such as RNaseHII), ultraviolet light, and a combination thereof.

According to some embodiments, the capture entity comprises one or more members selected from the group consisting of biotin, avidin, a nucleic acid sequence, a charged particle, and a magnetic particle.

According to some embodiments, the capture entity comprises biotin.

According to some embodiments, the capture entity comprises a nucleic acid sequence.

According to some embodiments, the capture entity comprises a magnetic or charged particle.

According to some embodiments, the capturing entity comprises one or more members selected from the group consisting of biotin, avidin, a nucleic acid sequence, an electric field system, and a magnetic field system.

According to some embodiments, the capturing entity is coupled to another support.

According to some embodiments, the other support is a particle.

According to some embodiments, the support is a particle.

According to some embodiments, the nucleic acid molecule comprises a deoxyribonucleic acid (DNA).

According to some embodiments, the nucleic acid molecule comprises a ribonucleic acid (RNA).

According to some embodiments, the nucleic acid molecule comprises an additional cleavable or excisable moiety.

According to some embodiments, the additional cleavable or excisable moiety is separated from the cleavable or excisable moiety by at least ten bases.

According to some embodiments, the additional cleavable or excisable moiety is separated from the cleavable or excisable moiety by fewer than ten bases.

According to some embodiments, the nucleic acid molecule comprises or is coupled to an additional capture moiety.

According to some embodiments, the method further comprises: (e) bringing the nucleic acid molecule coupled to the support into contact with an additional capturing entity under conditions sufficient to couple the additional capture entity and the additional capturing entity, thereby coupling the nucleic acid molecule to the additional capturing entity; (f) separating the nucleic acid molecule coupled to the additional capturing entity from other components of a solution comprising the nucleic acid molecule; and (g) subsequent to (f), subjecting the nucleic acid molecule coupled to the additional capturing entity to conditions sufficient to cleave or excise the additional cleavable or excisable moiety, thereby uncoupling the nucleic acid molecule and the additional capturing entity.

According to some embodiments, the additional capturing entity is coupled to a further support.

According to some embodiments, (g) comprises bringing the nucleic acid molecule in contact with a cleaving agent.

According to some embodiments, the cleaving agent is selected from the group consisting of uracil DNA glycosylase (UDG), apyrimidinic/apurinic endonuclease (APE), endonucleases (e.g., endonuclease VIII (EndoVIII) or V (EndoV)), uracil-specific excision reagent (USER) enzyme, formamidopyrimidine DNA glycosylase (Fpg), 8-oxoguanine glycosylase (OGG1), RNase (e.g., RNaseH, such as RNaseHII), ultraviolet light, and a combination thereof.

According to some embodiments, the method further comprises subjecting the nucleic acid molecule to conditions sufficient to cleave or excise the additional cleavable or excisable moiety, thereby generating a nick or gap region in the first strand of the nucleic acid molecule.

According to some embodiments, subjecting the nucleic acid molecule to conditions sufficient to cleave or excise the additional cleavable or excisable moiety comprises bringing the nucleic acid molecule in contact with a cleaving agent.

According to some embodiments, the cleaving agent is selected from the group consisting of uracil DNA glycosylase (UDG), apyrimidinic/apurinic endonuclease (APE), endonucleases (e.g., endonuclease VIII (EndoVIII) or V (EndoV)), uracil-specific excision reagent (USER) enzyme, formanidopyrimidine DNA glycosylase (Fpg), 8-oxoguanine glycosylase (OGG1), RNase (e.g., RNaseH, such as RNaseHII), ultraviolet light, and a combination thereof.

According to some embodiments, cleavage or excision of the additional cleavable or excisable moiety generates a gap region of two or more bases in the first strand of the nucleic acid molecule.

According to some embodiments, the method further comprises bringing the nucleic acid molecule comprising the nick or gap region into contact with a polymerase enzyme and a labeled nucleotide, wherein the labeled nucleotide is configured to emit a signal.

According to some embodiments, the method further comprises subjecting the nucleic acid molecule comprising the nick or gap region to conditions sufficient to incorporate the labeled nucleotide into the nick or gap region.

According to some embodiments, the method further comprises detecting a signal or change in signal from the labeled nucleotide incorporated into the nick or gap region of the nucleic acid molecule, wherein the signal or the change in signal is indicative of incorporation of the labeled nucleotide into the nick or gap region of the nucleic acid molecule.

According to some embodiments, the polymerase enzyme is a strand-displacing polymerase enzyme.

According to some embodiments, the labeled nucleotide comprises a fluorescent dye.

According to another aspect, there is provided a method of analyzing a double-stranded nucleic acid molecule, the method comprising:
a) generating the double-stranded nucleic acid molecule comprising a strand comprising a target nucleic acid sequence and a nick region 5′ to the target nucleic acid sequence from a nucleic acid molecule coupled to a support, wherein the nucleic acid molecule coupled to the support comprises (i) a first strand that comprises at least two cleavable or excisable moieties and comprises or is coupled to a capture entity and (ii) a second strand;
b) contacting the double-stranded nucleic acid molecule with a polymerase enzyme, and a plurality of nucleotides comprising a plurality of labeled nucleotides, under conditions sufficient to incorporate a labeled nucleotide of the plurality of labeled nucleotides into a position adjacent to a 5′ end of the nick region; and
c) detecting a signal or change in signal from the double-stranded nucleic acid molecule, wherein the signal or change in signal is indicative of incorporation of the labeled nucleotide into the nick region.

According to some embodiments, the double-stranded nucleic acid molecule is a ribonucleic acid molecule.

According to some embodiments, the double-stranded nucleic acid molecule is a deoxyribonucleic acid molecule.

According to some embodiments, the nick region comprises a single base gap.

According to some embodiments, the nick region comprises a gap region of two or more bases.

According to some embodiments, the nick region comprises a gap region of five or fewer bases.

According to some embodiments, the polymerase enzyme is a strand-displacement polymerase enzyme.

According to some embodiments, each nucleotide of the plurality of nucleotides comprises a nucleobase of a same type.

According to some embodiments, at least 20% of nucleotides of the plurality of nucleotides are labeled nucleotides.

According to some embodiments, each nucleotide of the plurality of nucleotides is a labeled nucleotide.

According to some embodiments, the labeled nucleotide comprises a fluorescent dye.

According to some embodiments, the plurality of nucleotides is a plurality of non-terminated nucleotides.

According to some embodiments, the labeled nucleotide is configured to emit a signal.

According to some embodiments, the method further comprises repeating (b) with an additional plurality of nucleotides.

According to some embodiments, each nucleotide of the plurality of nucleotides and the additional plurality of nucleotides comprises a nucleobase of a same type.

According to some embodiments, each nucleotide of the additional plurality of nucleotides is an unlabeled nucleotide.

According to some embodiments, a nucleotide of the plurality of nucleotides and a nucleotide of the additional plurality of nucleotides each comprise nucleobases of different types.

According to some embodiments, the additional plurality of nucleotides comprises an additional plurality of labeled nucleotides.

According to some embodiments, at least 20% of nucleotides of the additional plurality of nucleotides are labeled nucleotides.

According to some embodiments, each nucleotide of the additional plurality of nucleotides is a labeled nucleotide.

T According to some embodiments, (b) is repeated at least three times.

According to some embodiments, the method further comprises repeating (c).

According to some embodiments, the method further comprises, subsequent to (b), contacting the double-stranded nucleic acid molecule with a washing solution to remove unincorporated nucleotides of the plurality of nucleotides.

According to some embodiments, the support is a particle.

According to some embodiments, the double-stranded nucleic acid molecule is coupled to an additional support.

According to some embodiments, the additional support is a particle.

According to some embodiments, the additional support comprises a clonal population of double-stranded nucleic acid molecules, wherein each double-stranded nucleic acid molecule of the clonal population comprises a strand comprising the target nucleic acid sequence and a nick 5′ to the target nucleic acid sequence.

According to some embodiments, (b) is performed using a plurality of double-stranded nucleic acid molecules in parallel, wherein the plurality of double-stranded nucleic acid molecules comprises a plurality of different target nucleic acid sequences.

According to some embodiments, (a) comprises contacting the nucleic acid molecule coupled to the support with a cleaving agent configured to cleave or excise the at least two cleavable or excisable moieties.

According to some embodiments, the nucleic acid molecule coupled to the support is coupled to a further support via a capturing entity coupled to the capture entity, wherein generating the double-stranded nucleic acid molecule from the nucleic acid molecule coupled to the support uncouples the nucleic acid molecule from the support.

According to some embodiments, the capturing entity comprises a member selected from the group consisting of biotin, avidin, a nucleic acid sequence, an electric field system, and a magnetic field system.

According to some embodiments, the further support is a particle.

According to some embodiments, the at least two cleavable or excisable moieties are selected from the group consisting of a ribonucleic acid (RNA) base, a uracil base, an inosine base, 2,6-diamino-4-hydroxy-5-formamidopyrimidine (FapyG) base, 8-oxo-7,8-dihydroguanine (8oxoG) base, and a photocleavable base.

According to some embodiments, two or more of the at least two cleavable or excisable moieties are of a same type.

According to some embodiments, two or more of the at least two cleavable or excisable moieties are of different types.

According to some embodiments, two or more of the at least two cleavable or excisable moieties are separated by three or fewer bases.

According to some embodiments, the capture entity comprises a member selected from the group consisting of biotin, avidin, a nucleic acid sequence, a charged particle, and a magnetic particle.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1A shows an exemplary composition including a cleavable base at the 5′-most position. The lower panel shows the composition after excision of the cleavable base.

FIG. 1B shows an exemplary composition including a sequence with a cleavable base proximal to the 5′-most position. Below the solid line is depicted the dissociation of the 5′ proximal bases after excision of the cleavable base.

FIGS. 2A-2B shows exemplary methods for producing a composition provided herein.

FIG. 2C shows an exemplary method for producing duplex template molecules with different adapters at each end.

FIGS. 3A-3C show an exemplary pre-enrichment method provided herein.

FIGS. 4A-4C show an exemplary post-enrichment method provided herein.

FIGS. 4D-4E show exemplary alternatives to FIG. 4A in which a pre-enriched complex is added as the input for amplification.

FIGS. 5A-5B show an example of a combined post-enrichment and sequencing method provided herein.

FIGS. 5C-5E show an example of combined post-enrichment and pair-end sequencing method provided herein.

FIGS. 6A-6D show an example of pair-end sequencing with a single cleavable or excisable base as provided herein.

FIGS. 6E-6G show an example of pair-end sequencing with a plurality of cleavable or excisable bases as provided herein.

FIG. 7 shows an exemplary architecture of a computer system programmed or otherwise configured to implement methods provided herein.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

The invention is based on the surprising finding of ways to improve clonal amplification of template nucleic acid molecules that avoid wasted reagents and lost sample by circumventing the double-Poisson distribution problem inherent is clonal amplification. Amplification performed in partitions requires the distribution of nucleic acid templates and amplification supports (e.g., beads) to the various partitions. Standard amplification calls for a single bead and a single template to be present in a partition to facilitate the production of a clonal bead bound by amplification products homologous or complementary to the template nucleic acid. When a partition contains only a bead, only a nucleic acid, or neither no amplification can occur and the reagents in the partition are wasted. Further, precious nucleic acid templates with no bead are also lost. Partitions with more than one nucleic acid produce a polyclonal bead which cannot be properly analyzed also resulting in wasted reagents and template. For a given case of “N” number of nucleic acid molecules and “B” number of beads randomly distributed among partitions which are greatly in excess, the relative bead population found in partitions with any number of DNAs (0, 1 or >1 nucleic acid molecules) is dependent on the ratio of N/M. When beads and temple nucleic acids are distributed into partitions separately each will follow its own Poisson distribution leading to a double-Poisson problem. The fraction of beads containing N number of nucleic acids, R(N) may be calculated as: R(N)=e\hat{ }−(N/M)×(N/M)\hat{ }N/N!

In order to maximize partitions with only one bead and only one nucleic acid template an N/M ratio of 1 would be selected. In such a case 37% of beads will be alone in a partition, 26% of beads will be in partitions with more than one template and 37% of beads will be in partitions with a single template. This is already a large loss of template. However, due to the double-Poisson issue the situation is even worse. Of those partitions with only a single template molecule some will have multiple beads, so the percentage of nucleic acids in partitions with a single bead is even less than 37%, and indeed approximately 22%. Similarly, only 22% of template molecules will be in partitions with a single bead and single template. Herein is provided a method of pre-enrichment in which a template molecule with a capture entity is linked to an amplification bead. The beads with a bound copy of the template molecule are isolated with a capturing agent and then released. In this way the input for the amplification is always a single bead and a single template. Template which did not bind to a bead (i.e., went to an empty partition) may be readministered to more beads which can be again isolated. This may be repeated as many times as needed so that precious sample is not lost. Since all beads in the amplification have bound nucleic acids before distribution to the partitions one of the Poisson distributions is removed. The use of cleavable or excisable bases provides an elegant way to remove the capturing agent after pre-enrichment and can be combined with amplification process.

In invention is also based on the surprising finding of the superiority of employing double-stranded templates. Single-stranded template is generally used as the input for clonal amplification. However, when single-stranded template was used for pre-enrichment the single-strands tended to hybridize to each other, and to themselves, leading to variable ligation and hybridization efficiency. This leads to dropping out of some targets and overall loss of read quality and sequencing efficiency/accuracy and total genome coverage. Double-stranded templates decrease inter-bead binding, eliminate internal secondary structure formation allow for multiple methods of disengagement from the capturing entity and provide novel methods of sequence analysis (as described herein below). One particular beneficial finding is that with the use of double-stranded molecules a post-enrichment step (post-amplification) can be combined with the sequencing by a single step of removal of cleavable/excisable bases. Double-stranded molecules during post-enrichment are particularly beneficial. Amplified beads with single-stranded molecules tend to clump and aggregate due to inter-bead hybridization of single-stranded molecules. The close proximity of beads due to the enrichment greatly exacerbates this problem. Double-stranded amplification products, however, cannot create these inter-bead linkages, and thus are greatly less prone to clumping.

Where values are described as ranges, it will be understood that such disclosure includes the disclosure of all possible sub-ranges within such ranges, as well as specific numerical values that fall within such ranges irrespective of whether a specific numerical value or specific sub-range is expressly stated.

The terms “about” and “approximately” shall generally mean an acceptable degree of error or variation for a given value or range of values, such as, for example, a degree of error or variation that is within 20 percent (%), within 15%, within 10%, or within 5% of a given value or range of values.

The term “at least partially” as used herein, generally refers to any fraction of a whole amount. For example, “at least partially” may refer to at least about 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 99.9%, or more of a whole amount.

The term “subject,” as used herein, generally refers to an individual or entity from which a biological sample (e.g., a biological sample that is undergoing or will undergo processing or analysis) may be derived. A subject may be an animal (e.g., mammal or non-mammal) or plant. The subject may be a human, dog, cat, horse, pig, bird, non-human primate, simian, farm animal, companion animal, sport animal, or rodent. A subject may be a patient. The subject may have or be suspected of having a disease or disorder, such as cancer (e.g., breast cancer, colorectal cancer, brain cancer, leukemia, lung cancer, skin cancer, liver cancer, pancreatic cancer, lymphoma, esophageal cancer or cervical cancer) or an infectious disease. Alternatively, or in addition to, a subject may be known to have previously had a disease or disorder. The subject may have or be suspected of having a genetic disorder such as achondroplasia, alpha-1 antitrypsin deficiency, antiphospholipid syndrome, autism, autosomal dominant polycystic kidney disease, Charcot-Marie-tooth, cri du chat, Crohn's disease, cystic fibrosis, Dercum disease, down syndrome, Duane syndrome, Duchenne muscular dystrophy, factor V Leiden thrombophilia, familial hypercholesterolemia, familial Mediterranean fever, fragile x syndrome, Gaucher disease, hemochromatosis, hemophilia, holoprosencephaly, Huntington's disease, Klinefelter syndrome, Marfan syndrome, myotonic dystrophy, neurofibromatosis, Noonan syndrome, osteogenesis imperfecta, Parkinson's disease, phenylketonuria, Poland anomaly, porphyria, progeria, retinitis pigmentosa, severe combined immunodeficiency, sickle cell disease, spinal muscular atrophy, Tay-Sachs, thalassemia, trimethylaminuria, Turner syndrome, velocardiofacial syndrome, WAGR syndrome, or Wilson disease. A subject may be undergoing treatment for a disease or disorder. A subject may be symptomatic or asymptomatic of a given disease or disorder. A subject may be healthy (e.g., not suspected of having disease or disorder). A subject may have one or more risk factors for a given disease. A subject may have a given weight, height, body mass index, or other physical characteristic. A subject may have a given ethnic or racial heritage, place of birth or residence, nationality, disease or remission state, family medical history, or other characteristic.

As used herein, the term “biological sample” generally refers to a sample obtained from a subject. The biological sample may be obtained directly or indirectly from the subject. A sample may be obtained from a subject via any suitable method, including, but not limited to, spitting, swabbing, blood draw, biopsy, obtaining excretions (e.g., urine, stool, sputum, vomit, or saliva), excision, scraping, and puncture. A sample may be obtained from a subject by, for example, intravenously or intraarterially accessing the circulatory system, collecting a secreted biological sample (e.g., stool, urine, saliva, sputum, etc.), breathing, or surgically extracting a tissue (e.g., biopsy). The sample may be obtained by non-invasive methods including but not limited to: scraping of the skin or cervix, swabbing of the cheek, or collection of saliva, urine, feces, menses, tears, or semen. Alternatively, the sample may be obtained by an invasive procedure such as biopsy, needle aspiration, or phlebotomy. A sample may comprise a bodily fluid such as, but not limited to, blood (e.g., whole blood, red blood cells, leukocytes or white blood cells, platelets), plasma, serum, sweat, tears, saliva, sputum, urine, semen, mucus, synovial fluid, breast milk, colostrum, amniotic fluid, bile, bone marrow, interstitial or extracellular fluid, or cerebrospinal fluid. For example, a sample may be obtained by a puncture method to obtain a bodily fluid comprising blood and/or plasma. Such a sample may comprise both cells and cell-free nucleic acid material. Alternatively, the sample may be obtained from any other source including but not limited to blood, sweat, hair follicle, buccal tissue, tears, menses, feces, or saliva. The biological sample may be a tissue sample, such as a tumor biopsy. The sample may be obtained from any of the tissues provided herein including, but not limited to, skin, heart, lung, kidney, breast, pancreas, liver, intestine, brain, prostate, esophagus, muscle, smooth muscle, bladder, gall bladder, colon, or thyroid. The methods of obtaining provided herein include methods of biopsy including fine needle aspiration, core needle biopsy, vacuum assisted biopsy, large core biopsy, incisional biopsy, excisional biopsy, punch biopsy, shave biopsy or skin biopsy. The biological sample may comprise one or more cells. A biological sample may comprise one or more nucleic acid molecules such as one or more deoxyribonucleic acid (DNA) and/or ribonucleic acid (RNA) molecules (e.g., included within cells or not included within cells). Nucleic acid molecules may be included within cells. Alternatively, or in addition to, nucleic acid molecules may not be included within cells (e.g., cell-free nucleic acid molecules). The biological sample may be a cell-free sample.

The term “cell-free sample,” as used herein, generally refers to a sample that is substantially free of cells (e.g., less than 10% cells on a volume basis). A cell-free sample may be derived from any source (e.g., as described herein). For example, a cell-free sample may be derived from blood, sweat, urine, or saliva. For example, a cell-free sample may be derived from a tissue or bodily fluid. A cell-free sample may be derived from a plurality of tissues or bodily fluids. For example, a sample from a first tissue or fluid may be combined with a sample from a second tissue or fluid (e.g., while the samples are obtained or after the samples are obtained). In an example, a first fluid and a second fluid may be collected from a subject (e.g., at the same or different times) and the first and second fluids may be combined to provide a sample. A cell-free sample may comprise one or more nucleic acid molecules such as one or more DNA or RNA molecules.

A sample that is not a cell-free sample (e.g., a sample comprising one or more cells) may be processed to provide a cell-free sample. For example, a sample that includes one or more cells as well as one or more nucleic acid molecules (e.g., DNA and/or RNA molecules) not included within cells (e.g., cell-free nucleic acid molecules) may be obtained from a subject. The sample may be subjected to processing (e.g., as described herein) to separate cells and other materials from the nucleic acid molecules not included within cells, thereby providing a cell-free sample (e.g., comprising nucleic acid molecules not included within cells). The cell-free sample may then be subjected to further analysis and processing (e.g., as provided herein). Nucleic acid molecules not included within cells (e.g., cell-free nucleic acid molecules) may be derived from cells and tissues. For example, cell-free nucleic acid molecules may derive from a tumor tissue or a degraded cell (e.g., of a tissue of a body). Cell-free nucleic acid molecules may comprise any type of nucleic acid molecules (e.g., as described herein). Cell-free nucleic acid molecules may be double-stranded, single-stranded, or a combination thereof. Cell-free nucleic acid molecules may be released into a bodily fluid through secretion or cell death processes, e.g., cellular necrosis, apoptosis, or the like. Cell-free nucleic acid molecules may be released into bodily fluids from cancer cells (e.g., circulating tumor DNA (ctDNA)). Cell free nucleic acid molecules may also be fetal DNA circulating freely in a maternal blood stream (e.g., cell-free fetal nucleic acid molecules such as cffDNA). Alternatively, or in addition to, cell-free nucleic acid molecules may be released into bodily fluids from healthy cells.

A biological sample may be obtained directly from a subject and analyzed without any intervening processing, such as, for example, sample purification or extraction. For example, a blood sample may be obtained directly from a subject by accessing the subject's circulatory system, removing the blood from the subject (e.g., via a needle), and transferring the removed blood into a receptacle. The receptacle may comprise reagents (e.g., anti-coagulants) such that the blood sample is useful for further analysis. Such reagents may be used to process the sample or analytes derived from the sample in the receptacle or another receptacle prior to analysis. In another example, a swab may be used to access epithelial cells on an oropharyngeal surface of the subject. Following obtaining the biological sample from the subject, the swab containing the biological sample may be contacted with a fluid (e.g., a buffer) to collect the biological fluid from the swab.

Any suitable biological sample that comprises one or more nucleic acid molecules may be obtained from a subject. A sample (e.g., a biological sample or cell-free biological sample) suitable for use according to the methods provided herein may be any material comprising tissues, cells, degraded cells, nucleic acids, genes, gene fragments, expression products, gene expression products, and/or gene expression product fragments of an individual to be tested. A biological sample may be solid matter (e.g., biological tissue) or may be a fluid (e.g., a biological fluid). In general, a biological fluid may include any fluid associated with living organisms. Non-limiting examples of a biological sample include blood (or components of blood—e.g., white blood cells, red blood cells, platelets) obtained from any anatomical location (e.g., tissue, circulatory system, bone marrow) of a subject, cells obtained from any anatomical location of a subject, skin, heart, lung, kidney, breath, bone marrow, stool, semen, vaginal fluid, interstitial fluids derived from tumorous tissue, breast, pancreas, cerebral spinal fluid, tissue, throat swab, biopsy, placental fluid, amniotic fluid, liver, muscle, smooth muscle, bladder, gall bladder, colon, intestine, brain, cavity fluids, sputum, pus, microbiota, meconium, breast milk, prostate, esophagus, thyroid, serum, saliva, urine, gastric and digestive fluid, tears, ocular fluids, sweat, mucus, earwax, oil, glandular secretions, spinal fluid, hair, fingernails, skin cells, plasma, nasal swab or nasopharyngeal wash, spinal fluid, cord blood, emphatic fluids, and/or other excretions or body tissues. Methods for determining sample suitability and/or adequacy are provided. A sample may include, but is not limited to, blood, plasma, tissue, cells, degraded cells, cell-free nucleic acid molecules, and/or biological material from cells or derived from cells of an individual such as cell-free nucleic acid molecules. The sample may be a heterogeneous or homogeneous population of cells, tissues, or cell-free biological material. The biological sample may be obtained using any method that can provide a sample suitable for the analytical methods described herein.

A sample (e.g., a biological sample or cell-free biological sample) may undergo one or more processes in preparation for analysis, including, but not limited to, filtration, centrifugation, selective precipitation, permeabilization, isolation, agitation, heating, purification, and/or other processes. For example, a sample may be filtered to remove contaminants or other materials. In an example, a sample comprising cells may be processed to separate the cells from other material in the sample. Such a process may be used to prepare a sample comprising only cell-free nucleic acid molecules. Such a process may consist of a multi-step centrifugation process. Multiple samples, such as multiple samples from the same subject (e.g., obtained in the same or different manners from the same or different bodily locations, and/or obtained at the same or different times (e.g., seconds, minutes, hours, days, weeks, months, or years apart)) or multiple samples from different subjects may be obtained for analysis as described herein. In an example, the first sample is obtained from a subject before the subject undergoes a treatment regimen or procedure and the second sample is obtained from the subject after the subject undergoes the treatment regimen or procedure. Alternatively, or in addition to, multiple samples may be obtained from the same subject at the same or approximately the same time. Different samples obtained from the same subject may be obtained in the same or different manner. For example, a first sample may be obtained via a biopsy and a second sample may be obtained via a blood draw. Samples obtained in different manners may be obtained by different medical professionals, using different techniques, at different times, and/or at different locations. Different samples obtained from the same subject may be obtained from different areas of a body. For example, a first sample may be obtained from a first area of a body (e.g., a first tissue) and a second sample may be obtained from a second area of the body (e.g., a second tissue).

A biological sample as used herein (e.g., a biological sample comprising one or more nucleic acid molecules) may not be purified when provided in a reaction vessel. Furthermore, for a biological sample comprising one or more nucleic acid molecules, the one or more nucleic acid molecules may not be extracted when the biological sample is provided to a reaction vessel. For example, ribonucleic acid (RNA) and/or deoxyribonucleic acid (DNA) molecules of a biological sample may not be extracted from the biological sample when providing the biological sample to a reaction vessel. Moreover, a target nucleic acid (e.g., a target RNA or target DNA molecules) present in a biological sample may not be concentrated when providing the biological sample to a reaction vessel. Alternatively, a biological sample may be purified and/or nucleic acid molecules may be isolated from other materials in the biological sample.

A biological sample as described herein may contain a target nucleic acid. As used herein, the terms “template nucleic acid”, “target nucleic acid”, “nucleic acid molecule,” “nucleic acid sequence,” “nucleic acid fragment,” “oligonucleotide,” “polynucleotide,” and “nucleic acid” generally refer to polymeric forms of nucleotides of any length, such as deoxyribonucleotides (dNTPs) or ribonucleotides (rNTPs), or analogs thereof, and may be used interchangeably. Nucleic acids may have any three-dimensional structure, and may perform any function, known or unknown. A nucleic acid molecule may have a length of at least about 10 nucleic acid bases (“bases”), 20 bases, 30 bases, 40 bases, 50 bases, 100 bases, 200 bases, 300 bases, 400 bases, 500 bases, 1 kilobase (kb), 2 kb, 3, kb, 4 kb, 5 kb, 10 kb, 50 kb, or more. An oligonucleotide is typically composed of a specific sequence of four nucleotide bases: adenine (A); cytosine (C); guanine (G); and thymine (T) (uracil (U) for thymine (T) when the polynucleotide is RNA). Oligonucleotides may include one or more nonstandard nucleotide(s), nucleotide analog(s) and/or modified nucleotides. Non-limiting examples of nucleic acids include DNA, RNA, genomic DNA (e.g., gDNA such as sheared gDNA), cell-free DNA (e.g., cfDNA), synthetic DNA/RNA, coding or non-coding regions of a gene or gene fragment, loci (locus) defined from linkage analysis, exons, introns, messenger RNA (mRNA), transfer RNA, ribosomal RNA, short interfering RNA (siRNA), short-hairpin RNA (shRNA), micro-RNA (miRNA), ribozymes, complementary DNA (cDNA), recombinant nucleic acids, branched nucleic acids, plasmids, vectors, isolated DNA of any sequence, isolated RNA of any sequence, nucleic acid probes, and primers. A nucleic acid may comprise one or more modified nucleotides, such as methylated nucleotides and nucleotide analogs. If present, modifications to the nucleotide structure may be made before or following assembly of the nucleic acid. The sequence of nucleotides of a nucleic acid may be interrupted by non-nucleotide components. A nucleic acid may be further modified following polymerization, such as by conjugation or binding with a reporter agent.

A target nucleic acid or sample nucleic acid as described herein may be amplified to generate an amplified product. A target nucleic acid may be a target RNA or a target DNA. When the target nucleic acid is a target RNA, the target RNA may be any type of RNA, including types of RNA described elsewhere herein. The target RNA may be viral RNA and/or tumor RNA. A viral RNA may be pathogenic to a subject. Non-limiting examples of pathogenic viral RNA include human immunodeficiency virus I (HIV I), human immunodeficiency virus n (HIV 11), orthomyxoviruses, Ebola virus. Dengue virus, influenza viruses (e.g., H1N1, H3N2, H7N9, or H5N1), herpesvirus, hepatitis A virus, hepatitis B virus, hepatitis C (e.g., armored RNA-HCV virus) virus, hepatitis D virus, hepatitis E virus, hepatitis G virus, Epstein-Barr virus, mononucleosis virus, cytomegalovirus. SARS virus, West Nile Fever virus, polio virus, and measles virus.

A biological sample may comprise a plurality of target nucleic acid molecules. For example, a biological sample may comprise a plurality of target nucleic acid molecules from a single subject. In another example, a biological sample may comprise a first target nucleic acid molecule from a first subject and a second target nucleic acid molecule from a second subject.

As used herein, a “double-stranded” molecule is a molecule comprising a region of double-stranded nucleic acid molecule. In some embodiments, double-stranded is defined as a molecule that is 100% double-stranded. In some embodiments, double-stranded is defined as a molecule that is at least 50, 55, 60, 65, 70, 75, 80, 85, 90, 92, 95, 97, 99 or 100% double-stranded. Each possibility represents a separate embodiment of the invention. In some embodiments, a double-stranded molecule comprises a region of double-stranded nucleotides that is at least 1, 2, 3, 4, 5, 6, 7, 8, 9, 10, 12, 14, 15, 16, 18, 20, 25, 30, 35, 40, 45 or 50 bases long. Each possibility represents a separate embodiment of the invention. In some embodiments, the double-stranded molecule comprises a single-stranded overhang. In some embodiments, the overhang is not more than 1, 2, 3, 4, 5, 6, 7, 8, 9, 10, 11, 12, 13, 14, 15, 16, 17, 18, 19, or 20 bases in length. Each possibility represents a separate embodiment of the invention. In some embodiments, the overhang is at least 5, 6, 7, 8, 9, 10, 11, 12, 13, 14, 15, 16, 17, 18, 19, or 20 bases in length. Each possibility represents a separate embodiment of the invention.

The term “nucleotide,” as used herein, generally refers to a substance including a base (e.g., a nucleobase), sugar moiety, and phosphate moiety. A nucleotide may comprise a free base with attached phosphate groups. A substance including a base with three attached phosphate groups may be referred to as a nucleoside triphosphate. When a nucleotide is being added to a growing nucleic acid molecule strand, the formation of a phosphodiester bond between the proximal phosphate of the nucleotide to the growing chain may be accompanied by hydrolysis of a high-energy phosphate bond with release of the two distal phosphates as a pyrophosphate. The nucleotide may be naturally occurring or non-naturally occurring (e.g., a modified or engineered nucleotide).

The term “nucleotide analog,” as used herein, may include, but is not limited to, a nucleotide that may or may not be a naturally occurring nucleotide. For example, a nucleotide analog may be derived from and/or include structural similarities to a canonical nucleotide such as adenine- (A), thymine- (T), cytosine- (C), uracil- (U), or guanine- (G) including nucleotide. A nucleotide analog may comprise one or more differences or modifications relative to a natural nucleotide. Examples of nucleotide analogs include inosine, diaminopurine, 5-fluorouracil, 5-bromouracil, 5-chlorouracil, 5-iodouracil, hypoxanthine, xanthine, deazaxanthine, deazaguanine, isocytosine, isoguanine, 4-acetylcytosine, 5-(carboxyhydroxylmethyl)uracil, 5-carboxymethylaminomethyl-2-thiouridine, 5-carboxymethylaminomethyluracil, dihydrouracil, beta-D-galactosylqueosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethvluracil, 5-methoxyuracil, 2-methylthio-D46-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, 2,6-diaminopurine, ethynyl nucleotide bases, 1-propynyl nucleotide bases, azido nucleotide bases, phosphoroselenoate nucleic acids, and modified versions thereof (e.g., by oxidation, reduction, and/or addition of a substituent such as an alkyl, hydroxyalkyl, hydroxyl, or halogen moiety). Nucleic acid molecules (e.g., polynucleotides, double-stranded nucleic acid molecules, single-stranded nucleic acid molecules, primers, adapters, etc.) may be modified at the base moiety (e.g., at one or more atoms that typically are available to form a hydrogen bond with a complementary nucleotide and/or at one or more atoms that are not typically capable of forming a hydrogen bond with a complementary nucleotide), sugar moiety, or phosphate backbone. In some cases, a nucleotide may include a modification in its phosphate moiety, including a modification to a triphosphate moiety. Additional, non-limiting examples of modifications include phosphate chains of greater length (e.g., a phosphate chain having, 4, 5, 6, 7, 8, 9, 10 or more phosphate moieties), modifications with thiol moieties (e.g., alpha-thio triphosphate and beta-thiotriphosphates), and modifications with selenium moieties (e.g., phosphoroselenoate nucleic acids). A nucleotide or nucleotide analog may comprise a sugar selected from the group consisting of ribose, deoxyribose, and modified versions thereof (e.g., by oxidation, reduction, and/or addition of a substituent such as an alkyl, hydroxyalkyl, hydroxyl, or halogen moiety). A nucleotide analog may also comprise a modified linker moiety (e.g., in lieu of a phosphate moiety). Nucleotide analogs may also contain amine-modified groups, such as aminoallyl-dUTP (aa-dUTP) and aminohexhylacrylamide-dCTP (aha-dCTP) to allow covalent attachment of amine reactive moieties, such as N-hydroxysuccinimide esters (NHS). Alternatives to standard DNA base pairs or RNA base pairs in the oligonucleotides of the present disclosure may provide, for example, higher density in bits per cubic mm, higher safety (resistant to accidental or purposeful synthesis of natural toxins), easier discrimination in photo-programmed polymerases, and/or lower secondary structure. Nucleotide analogs may be capable of reacting or bonding with detectable moieties for nucleotide detection. An analog to a cleavable base may be the non-cleavable alternative to the base. For example, thymine is a non-cleavable analog to uracil and adenine is a non-cleavable analog of inosine.

The term “homopolymer,” as used herein, generally refers to a polymer or a portion of a polymer comprising identical monomer units. A homopolymer may have a homopolymer sequence. A nucleic acid homopolymer may refer to a polynucleotide or an oligonucleotide comprising consecutive repetitions of a same nucleotide or any nucleotide variants thereof. For example, a homopolymer can be poly(dA), poly(dT), poly(dG), poly(dC), poly(rA), poly(U), poly(rG), or poly(rC). A homopolymer can be of any length. For example, the homopolymer can have a length of at least 2, 3, 4, 5, 10, 20, 30, 40, 50, 100, 200, 300, 400, 500, or more nucleic acid bases. Each possibility represents a separate embodiment of the invention. The homopolymer can have from 10 to 500, or 15 to 200, or 20 to 150 nucleic acid bases. Each possibility represents a separate embodiment of the invention. The homopolymer can have a length of at most 500, 400, 300, 200, 100, 50, 40, 30, 20, 10, 5, 4, 3, or 2 nucleic acid bases. Each possibility represents a separate embodiment of the invention. A molecule, such as a nucleic acid molecule, can include one or more homopolymer portions and one or more non-homopolymer portions. The molecule may be entirely formed of a homopolymer, multiple homopolymers, or a combination of homopolymers and non-homopolymers. In nucleic acid sequencing, multiple nucleotides can be incorporated into a homopolymeric region of a nucleic acid strand. Such nucleotides may be non-terminated to permit incorporation of consecutive nucleotides (e.g., during a single nucleotide flow).

The terms “amplifying,” “amplification,” and “nucleic acid amplification” are used interchangeably and, as used herein, generally refer to the production of copies of a nucleic acid molecule. For example, “amplification” of DNA generally refers to generating one or more copies of a DNA molecule. In some embodiments, amplification is clonal amplification. An amplicon may be a single-stranded or double-stranded nucleic acid molecule that is generated by an amplification procedure from a starting template nucleic acid molecule. Such an amplification procedure may include one or more cycles of an extension or ligation procedure. The amplicon may comprise a nucleic acid strand, of which at least a portion may be substantially identical or substantially complementary to at least a portion of the starting template. Where the starting template is a double-stranded nucleic acid molecule, an amplicon may comprise a nucleic acid strand that is substantially identical to at least a portion of one strand and is substantially complementary to at least a portion of either strand. The amplicon can be single-stranded or double-stranded irrespective of whether the initial template is single-stranded or double-stranded. Amplification of a nucleic acid may linear, exponential, or a combination thereof. Amplification may be emulsion based or may be non-emulsion based. Non-limiting examples of nucleic acid amplification methods include reverse transcription, primer extension, polymerase chain reaction (PCR), ligase chain reaction (LCR), helicase-dependent amplification, asymmetric amplification, rolling circle amplification, and multiple displacement amplification (MDA). An amplification reaction may be, for example, a polymerase chain reaction (PCR), such as an emulsion polymerase chain reaction (emPCR; e.g., PCR carried out within a microreactor such as a well or droplet). Where PCR is used, any form of PCR may be used, with non-limiting examples that include real-time PCR, allele-specific PCR, assembly PCR, asymmetric PCR, digital PCR, emulsion PCR, dial-out PCR, helicase-dependent PCR, nested PCR, hot start PCR, inverse PCR, methylation-specific PCR, miniprimer PCR, multiplex PCR, nested PCR, overlap-extension PCR, thermal asymmetric interlaced PCR and touchdown PCR Moreover, amplification can be conducted in a reaction mixture comprising various components (e.g., a primer(s), template, nucleotides, a polymerase, buffer components, co-factors, etc.) that participate or facilitate amplification. In some cases, the reaction mixture comprises a buffer that permits context independent incorporation of nucleotides. Non-limiting examples include magnesium-ion, manganese-ion and isocitrate buffers. Additional examples of such buffers are described in Tabor, S. et al. C. C. PNAS, 1989, 86, 4076-4080 and U.S. Pat. Nos. 5,409,811 and 5,674,716, each of which is herein incorporated by reference in its entirety.

Amplification may be clonal amplification. The term “clonal,” as used herein, generally refers to a population of nucleic acids for which a substantial portion (e.g., greater than 50%, 60%, 70%, 80%, 90%, 95%, or 99%) of its members have substantially identical sequences (e.g., have sequences that are at least about 50%, 60%, 70%, 80%, 90%, 95%, or 99% identical to one another). Members of a clonal population of nucleic acid molecules may have sequence homology to one another. Such members may have sequence homology to a template nucleic acid molecule. In some instances, such members may have sequence homology to a complement of the template nucleic acid molecule (e.g., if single stranded). The members of the clonal population may be double-stranded or single-stranded. Members of a population may not be 100% identical or complementary because, e.g., “errors” may occur during the course of synthesis such that a minority of a given population may not have sequence homology with a majority of the population. For example, at least 50% of the members of a population may be substantially identical to each other or to a reference nucleic acid molecule (i.e., a molecule of defined sequence used as a basis for a sequence comparison). At least 60%, at least 70%, at least 80%, at least 90%, at least 95%, at least 99%, or more of the members of a population may be substantially identical to the reference nucleic acid molecule. Two molecules may be considered substantially identical (or homologous) if the percent identity between the two molecules is at least 60%, 70%, 75%, 80%, 85%, 90%, 95%, 98%, 99%, 99.9% or greater. Two molecules may be considered substantially complementary if the percent complementarity between the two molecules is at least 60%, 70%, 75%, 80%, 85%, 90%, 95%, 98%, 99%, 99.9% or greater. A low or insubstantial level of mixing of non-homologous nucleic acids may occur, and thus a clonal population may contain a minority of diverse nucleic acids (e.g., less than 30%, e.g., less than 10%).

Useful methods for clonal amplification from single molecules include rolling circle amplification (RCA) (Lizardi et al., Nat. Genet. 19:225-232 (1998), which is incorporated herein by reference), bridge PCR (Adams and Kron, Method for Performing Amplification of Nucleic Acid with Two Primers Bound to a Single Solid Support, Mosaic Technologies, Inc. (Winter Hill, Mass.); Whitehead Institute for Biomedical Research, Cambridge, Mass., (1997); Adessi et al., Nucl. Acids Res. 28:E87 (2000); Pemov et al., Nucl. Acids Res. 33:e11(2005); or U.S. Pat. No. 5,641,658, each of which is incorporated herein by reference), polony generation (Mitra et al., Proc. Natl. Acad. Sci. USA 100:5926-5931 (2003); Mitra et al., Anal. Biochem. 320:55-65(2003), each of which is incorporated herein by reference), and clonal amplification on beads using emulsions (Dressman et al., Proc. Natl. Acad. Sci. USA 100:8817-8822 (2003), which is incorporated herein by reference) or ligation to bead-based adapter libraries (Brenner et al., Nat. Biotechnol. 18:730-634 (2000); Brenner et al., Proc. Natl. Acad. Sci. USA 97:1665-1670 (2000)); Reinartz, et al., Brief Funct. Genomic Proteomic 1:95-104 (2002), each of which is incorporated herein by reference). The enhanced signal-to-noise ratio provided by clonal amplification more than outweighs the disadvantages of the cyclic sequencing requirement.

The term “polymerizing enzyme” or “polymerase,” as used herein, generally refers to any enzyme capable of catalyzing a polymerization reaction. A polymerizing enzyme may be used to extend a nucleic acid primer paired with a template strand by incorporation of nucleotides or nucleotide analogs. A polymerizing enzyme may add a new strand of DNA by extending the 3′ end of an existing nucleotide chain, adding new nucleotides matched to the template strand one at a time via the creation of phosphodiester bonds. The polymerase used herein can have strand displacement activity or non-strand displacement activity. Examples of polymerases include, without limitation, a nucleic acid polymerase. An example polymerase is a 029 DNA polymerase or a derivative thereof. A polymerase can be a polymerization enzyme. In some cases, a transcriptase or a ligase is used (i.e., enzymes which catalyze the formation of a bond). A polymerase may be naturally occurring or synthesized. A polymerase may have relatively high processivity, namely the capability of the polymerase to consecutively incorporate nucleotides into a nucleic acid template without releasing the nucleic acid template. Examples of polymerases include, but are not limited to, a DNA polymerase, an RNA polymerase, a thermostable polymerase, a wild-type polymerase, a modified polymerase, E. coli DNA polymerase I, T7 DNA polymerase, bacteriophage T4 DNA polymerase, Φ29 (phi29) DNA polymerase, Taq polymerase, Tth polymerase, Tli polymerase, Pfu polymerase, Pwo polymerase, VENT polymerase, DEEPVENT polymerase. EXTaq polymerase. LA-Taq polymerase. Sso polymerase, Poc polymerase, Pab polymerase, Mth polymerase, ES4 polymerase, Tru polymerase, Tac polymerase. Tne polymerase, Tma polymerase, Tea polymerase, Tih polymerase, Tfi polymerase, Platinum Taq polymerases, Tbr polymerase, Tfl polymerase, Pfu-turbo polymerase, Pyrobest polymerase, Pwo polymerase, KOD polymerase, Bst polymerase, Sac polymerase, Klenow fragment, polymerase with 3′ to 5′ exonuclease activity, and variants, modified products and derivatives thereof. A polymerase may be a single subunit polymerase. The polymerase can have high processivity, namely the capability of the polymerase to consecutively incorporate nucleotides into a nucleic acid template without releasing the nucleic acid template. In some cases, a polymerase is a polymerase modified to accept dideoxynucleotide triphosphates, such as for example, Taq polymerase having a 667Y mutation (see e.g., Tabor et al. PNAS, 1995, 92, 6339-6343, which is herein incorporated by reference in its entirety for all purposes). In some cases, a polymerase is a polymerase having a modified nucleotide binding, which may be useful for nucleic acid sequencing, with non-limiting examples that include ThermoSequenas polymerase (GE Life Sciences), AmpliTaq FS (ThermoFisher) polymerase and Sequencing Pol polymerase (Jena Bioscience). In some cases, the polymerase is genetically engineered to have discrimination against dideoxynucleotides, such as for example, Sequenase DNA polymerase (ThermoFisher). In some embodiments, the polymerase is a strand-displacing polymerase. In some embodiments, the polymerase is a polymerase enzyme.

A polymerase may be Family A polymerase or a Family B DNA polymerase. Family A polymerases include, for example, Taq, Klenow, and Bst polymerases. Family B polymerases include, for example, Vent(exo-) and Therminator polymerases. Family B polymerases are known to accept more varied nucleotide substrates than Family A polymerases. Family A polymerases are used widely in sequencing by synthesis methods, likely due to their high processivity and fidelity.

The term “complementary sequence,” as used herein, generally refers to a sequence that hybridizes to another sequence. Hybridization between two single-stranded nucleic acid molecules may involve the formation of a double-stranded structure that is stable under certain conditions. Two single-stranded polynucleotides may be considered to be hybridized if they are bonded to each other by two or more sequentially adjacent base pairings. A substantial proportion of nucleotides in one strand of a double-stranded structure may undergo Watson-Crick base-pairing with a nucleoside on the other strand. Hybridization may also include the pairing of nucleoside analogs, such as deoxyinosine, nucleosides with 2-aminopurine bases, and the like, that may be employed to reduce the degeneracy of probes, whether or not such pairing involves formation of hydrogen bonds.

The term “denaturation,” as used herein, generally refers to separation of a double-stranded molecule (e.g., DNA) into single-stranded molecules. Denaturation may be complete or partial denaturation. In partial denaturation, a single-stranded region may form in a double-stranded molecule by denaturation of the two deoxyribonucleic acid (DNA) strands flanked by double-stranded regions in DNA. Denaturation may be achieved by any method known in the art, including for example, through heating or addition of sodium hydroxide.

The term “melting temperature” or “melting point,” as used herein, generally refers to the temperature at which at least a portion of a strand of a nucleic acid molecule in a sample has separated from at least a portion of a complementary strand. The melting temperature may be the temperature at which a double-stranded nucleic acid molecule has partially or completely denatured. The melting temperature may refer to a temperature of a sequence among a plurality of sequences of a given nucleic acid molecule, or a temperature of the plurality of sequences. Different regions of a double-stranded nucleic acid molecule may have different melting temperatures. For example, a double-stranded nucleic acid molecule may include a first region having a first melting point and a second region having a second melting point that is higher than the first melting point. Accordingly, different regions of a double-stranded nucleic acid molecule may melt (e.g., partially denature) at different temperatures. The melting point of a nucleic acid molecule or a region thereof (e.g., a nucleic acid sequence) may be determined experimentally (e.g., via a melt analysis or other procedure) or may be estimated based upon the sequence and length of the nucleic acid molecule. For example, a software program such as MELTING may be used to estimate a melting temperature for a nucleic acid sequence (Dumousseau M, Rodriguez N, Juty N, Le Novère N, “MELTING, a flexible platform to predict the melting temperatures of nucleic acids.” BMC Bioinformatics. 2012 May 16:13:101. doi: 10.1186/1471-2105-13-101). Accordingly, a melting point as described herein may be an estimated melting point. A true melting point of a nucleic acid sequence may vary based upon the sequences or lack thereof adjacent to the nucleic acid sequence of interest as well as other factors.

The term “sequencing,” as used herein, generally refers to a process for generating or identifying a sequence of a biological molecule, such as a nucleic acid molecule or a polypeptide. Such sequence may be a nucleic acid sequence, which may include a sequence of nucleic acid bases (e.g., nucleobases). Sequencing may be, for example, single molecule sequencing, sequencing by synthesis, sequencing by hybridization, or sequencing by ligation. Sequencing may be performed using template nucleic acid molecules immobilized on a support, such as a flow cell or one or more beads. A sequencing assay may yield one or more sequencing reads corresponding to one or more template nucleic acid molecules.

The term “read,” as used herein, generally refers to a nucleic acid sequence, such as a sequencing read. A sequencing read may be an inferred sequence of nucleic acid bases (e.g., nucleotides) or base pairs obtained via a nucleic acid sequencing assay. A sequencing read may be generated by a nucleic acid sequencer, such as a massively parallel array sequencer (e.g., Illumina or Pacific Biosciences of California). A sequencing read may correspond to a portion, or in some cases all, of a genome of a subject. A sequencing read may be part of a collection of sequencing reads, which may be combined through, for example, alignment (e.g., to a reference genome), to yield a sequence of a genome of a subject.

The term “detector,” as used herein, generally refers to a device that is capable of detecting or measuring a signal, such as a signal indicative of the presence or absence of an incorporated nucleotide or nucleotide analog. A detector may include optical and/or electronic components that may detect and/or measure signals. Non-limiting examples of detection methods involving a detector include optical detection, spectroscopic detection, electrostatic detection, and electrochemical detection. Optical detection methods include, but are not limited to, fluorimetry and UV-vis light absorbance. Spectroscopic detection methods include, but are not limited to, mass spectrometry, nuclear magnetic resonance (NMR) spectroscopy, and infrared spectroscopy. Electrostatic detection methods include, but are not limited to, gel-based techniques, such as, for example, gel electrophoresis. Electrochemical detection methods include, but are not limited to, electrochemical detection of amplified product after high-performance liquid chromatography separation of the amplified products.

The term “support” or “substrate,” as used herein, generally refers to any solid or semi-solid article on which reagents such as nucleic acid molecules may be immobilized. Nucleic acid molecules may be synthesized, attached, ligated, or otherwise immobilized. Nucleic acid molecules may be immobilized on a substrate by any method including, but not limited to, physical adsorption, by ionic or covalent bond formation, or combinations thereof. A substrate may be 2-dimensional (e.g., a planar 2D substrate) or 3-dimensional. In some cases, a substrate may be a component of a flow cell and/or may be included within or adapted to be received by a sequencing instrument. A substrate may include a polymer, a glass, or a metallic material. Examples of substrates include a membrane, a planar substrate, a microtiter plate, a bead (e.g., a magnetic bead), a filter, a test strip, a slide, a cover slip, and a test tube. A substrate may comprise organic polymers such as polystyrene, polyethylene, polypropylene, polyfluoroethylene, polyethyleneoxy, and polyacrylamide (e.g., polyacrylamide gel), as well as co-polymers and grafts thereof. A substrate may comprise latex or dextran. A substrate may also be inorganic, such as glass, silica, gold, controlled-pore-glass (CPG), or reverse-phase silica. The configuration of a support may be, for example, in the form of beads, spheres, particles, granules, a gel, a porous matrix, or a substrate. In some cases, a substrate may be a single solid or semi-solid article (e.g., a single particle), while in other cases a substrate may comprise a plurality of solid or semi-solid articles (e.g., a collection of particles). Substrates may be planar, substantially planar, or non-planar. Substrates may be porous or non-porous and may have swelling or non-swelling characteristics. A substrate may be shaped to comprise one or more wells, depressions, or other containers, vessels, features, or locations. A plurality of substrates may be configured in an array at various locations. A substrate may be addressable (e.g., for robotic delivery of reagents), or by detection approaches, such as scanning by laser illumination and confocal or deflective light gathering. For example, a substrate may be in optical and/or physical communication with a detector. Alternatively, a substrate may be physically separated from a detector by a distance. An amplification substrate (e.g., a bead) can be placed within or on another substrate (e.g., within a well of a second support). In some embodiments, the support is an artificial support. In some embodiments, the support is a non-organic support. In some embodiments, the support is a bead.

The term “solid support” refers to any artificial solid structure, including any solid support or substrate. Examples of solid supports include, but are not limited to, beads, resins, gels, hydrogels, colloids, particles, or nanoparticles. For example, a solid support may be a bead. Alternatively, the solid support may be a surface. For example, a solid support may comprise a bead coupled to a surface. Alternatively, the solid support may be a resin. The solid support may be isolatable. The solid support may be tagged. The solid support may be magnetic and isolatable with a magnet. Alternatively, or in addition to, the solid support may be isolated by centrifugation or some other force that separates by weight, size, or some other measurable quantity.

A support (e.g., a solid support) may be or comprise a particle. A particle may be a bead. A bead may comprise any suitable material such as glass or ceramic, one or more polymers, and/or metals. Examples of suitable polymers include, but are not limited to, nylon, polytetrafluoroethylene, polystyrene, polyacrylamide, agarose, cellulose, cellulose derivatives, or dextran. Examples of suitable metals include paramagnetic metals, such as iron. A bead may be magnetic or non-magnetic. In some embodiments, the bead is magnetic. For example, a bead may comprise one or more polymers bearing one or more magnetic labels. A magnetic bead may be manipulated (e.g., moved between locations or physically constrained to a given location, e.g., of a reaction vessel such as a flow cell chamber) using electromagnetic forces. A bead may have any useful shape, including, for example, a shape that is approximately cubic, spherical, ellipsoidal, dumbbell-shaped, or any other shape. For example, a bead may be approximately spherical in shape. A bead may have one or more different dimensions including a diameter. A dimension of the bead (e.g., a diameter of the bead) may be less than about 1 mm, less than about 0.1 mm, less than about 0.01 mm, less than about 0.005 mm, less than about 1 nm, less than about 1 μm, or smaller. A dimension of the bead (e.g., a diameter of the bead) may be between about 1 nm to about 100 nm, about 1 μm to about 100 μm, about 1 mm to about 100 mm. A collection of beads may comprise one or more beads having the same or different characteristics. For example, a first bead of a collection of beads may have a first diameter and a second bead of the collection of beads may have a second diameter. The first diameter may be the same or approximately the same as or different from the second diameter. Similarly, the first bead may have the same or a different shape and composition than a second bead.

The term “label,” as used herein, generally refers to a moiety that is capable of coupling with a species, such as, for example a nucleotide analog. A label may include an affinity moiety. In some cases, a label may be a detectable label that emits a signal (or reduces an already emitted signal) that can be detected. In some cases, such a signal may be indicative of incorporation of one or more nucleotides or nucleotide analogs. In some cases, a label may be coupled to a nucleotide or nucleotide analog, which nucleotide or nucleotide analog may be used in a primer extension reaction. In some cases, the label may be coupled to a nucleotide analog after a primer extension reaction. The label, in some cases, may be reactive specifically with a nucleotide or nucleotide analog. Coupling may be covalent or non-covalent (e.g., via ionic interactions, Van der Waals forces, etc.). In some cases, coupling may be via a linker, which may be cleavable, such as photo-cleavable (e.g., cleavable under ultra-violet light), chemically-cleavable (e.g., via a reducing agent, such as dithiothreitol (DTT), tris(2-carboxyethyl)phosphine (TCEP), tris(hydroxypropyl)phosphine (THP) or enzymatically cleavable (e.g., via an esterase, lipase, peptidase or protease). In some cases, the label may be luminescent; that is, fluorescent or phosphorescent. For example, the label may be or comprise a fluorescent moiety (e.g., a dye). Dyes and labels may be incorporated into nucleic acid sequences. Dyes and labels may also be incorporated into linkers, such as linkers for linking one or more beads to one another. For example, labels such as fluorescent moieties may be linked to nucleotides or nucleotide analogs via a linker. Non-limiting examples of dyes include SYBR green, SYBR blue, DAPI, propidium iodine, Hoechst, SYBR gold, ethidium bromide, acridine, proflavine, acridine orange, acriflavine, fluorcoumanin, ellipticine, daunomycin, chloroquine, distamycin D, chromomycin, homidium, mithramycin, ruthenium polypyridyls, anthramycin, phenanthridines and acridines, propidium iodide, hexidium iodide, dihydroethidium, ethidium homodimer-1 and -2, ethidium monoazide, ACMA, Hoechst 33258, Hoechst 33342, Hoechst 34580, DAPI, acridine orange, 7-AAD, actinomycin D. LDS751, hydroxystilbamidine, SYTOX Blue, SYTOX Green, SYTOX Orange, POPO-1, POPO-3, YOYO-1, YOYO-3, TOTO-1, TOTO-3, JOJO-1, LOLO-1, BOBO-1, BOBO-3, PO-PRO-1, PO-PRO-3. BO-PRO-1, BO-PRO-3, TO-PRO-1, TO-PRO-3, TO-PRO-5, JO-PRO-1, LO-PRO-1, YO-PRO-1, YO-PRO-3, PicoGreen, OliGreen, RiboGreen, SYBR Gold, SYBR Green I, SYBR Green II, SYBR DX, SYTO labels (e.g., SYTO-40, -41, -42, -43, -44, and -45 (blue); SYTO-13, -16, -24, -21, -23, -12, -11, -20, -22, -15, -14, and -25 (green); SYTO-81, -80, -82, -83, -84, and -85 (orange); and SYTO-64, -17, -59, -61, -62, -60, and -63 (red)), fluorescein, fluorescein isothiocyanate (FITC), tetramethyl rhodamine isothiocyanate (TRITC), rhodamine, tetramethyl rhodamine, R-phycoerythrin, Cy-2, Cy-3, Cy-3.5, Cy-5, Cy5.5, Cy-7, Texas Red, Phar-Red, allophycocyanin (APC), Sybr Green I, Sybr Green II, Sybr Gold. CellTracker Green, 7-AAD, ethidium homodimer I, ethidium homodimer II, ethidium homodimer III, ethidium bromide, umbelliferone, eosin, green fluorescent protein, erythrosin, coumarin, methyl coumarin, pyrene, malachite green, stilbene, lucifer yellow, cascade blue, dichlorotriazinylamine fluorescein, dansyl chloride, fluorescent lanthanide complexes such as those including europium and terbium, carboxy tetrachloro fluorescein, 5 and/or 6-carboxy fluorescein (FAM), VIC, 5- (or 6-) iodoacetamidofluorescein, 5-{[2(and 3)-5-(Acetylmercapto)-succinyl]amino}fluorescein (SAMSA-fluorescein), lissamine diodamine B sulfonyl chloride, 5 and/or 6 carboxy rhodamine (ROX), 7-amino-methyl-coumarin, 7-Amino-4-methylcoumarin-3-acetic acid (AMCA), BODIPY fluorophores, 8-methoxypyrene-1,3,6-trisulfonic acid trisodium salt, 3,6-Disulfonate-4-amino-naphthalimide, phycobiliproteins, AlexaFluor labels (e.g., AlexaFluor 350, 405, 430, 488, 532, 546, 555, 568, 594, 710, 633, 735, 647, 660, 680, 700, 750, and 790 dyes), DyLight labels (e.g., DyLight 350, 405, 488, 550, 594, 633, 650, 680, 755, and 800 dyes), Black Hole Quencher Dyes (Biosearch Technologies) (e.g., BH1-0, BHQ-1, BHQ-3, and BHQ-10), QSY Dye fluorescent quenchers (Molecular Probes/Invitrogen) (e.g., QSY7, QSY9, QSY21, and QSY35), Dabcyl, Dabsy1, Cy5Q, Cy7Q, Dark Cyanine dyes (GE Healthcare), Dy-Quenchers (Dyomics) (e.g., DYQ-660 and DYQ-661). ATTO fluorescent quenchers (ATTO-TEC GmbH) (e.g., ATTO 540Q, ATTO 580Q, ATTO 612Q, Atto532 [e.g., Atto 532 succinimidyl ester], and Atto633), and other fluorophores and/or quenchers. A fluorescent dye may be excited by application of energy corresponding to the visible region of the electromagnetic spectrum (e.g., between about 430-770 nanometers (nm)). Excitation may be done using any useful apparatus, such as a laser and/or light emitting diode. Optical elements including, but not limited to, mirrors, waveplates, filters, monochromaters, gratings, beam splitters, and lenses may be used to direct light to or from a fluorescent dye. A fluorescent dye may emit light (e.g., fluoresce) in the visible region of the electromagnetic spectrum ((e.g., between about 430-770 nm). A fluorescent dye may be excited over a single wavelength or a range of wavelengths. A fluorescent dye may be excitable by light in the red region of the visible portion of the electromagnetic spectrum (about 725-740 nm)(e.g., have an excitation maximum in the red region of the visible portion of the electromagnetic spectrum). Alternatively or in addition to, fluorescent dye may be excitable by light in the green region of the visible portion of the electromagnetic spectrum (about 500-565 nm) (e.g., have an excitation maximum in the green region of the visible portion of the electromagnetic spectrum). A fluorescent dye may emit signal in the red region of the visible portion of the electromagnetic spectrum (about 725-740 nm) (e.g., have an emission maximum in the red region of the visible portion of the electromagnetic spectrum). Alternatively, or in addition to, fluorescent dye may emit signal in the green region of the visible portion of the electromagnetic spectrum (about 500-565 nm) (e.g., have an emission maximum in the green region of the visible portion of the electromagnetic spectrum).

Labels may be quencher molecules. The term “quencher,” as used herein refers to a molecule that may be energy acceptors. A quencher may be a molecule that can reduce an emitted signal. For example, a template nucleic acid molecule may be designed to emit a detectable signal. Incorporation of a nucleotide or nucleotide analog comprising a quencher can reduce or eliminate the signal, which reduction or elimination is then detected. Luminescence from labels (e.g., fluorescent moieties, such as fluorescent moieties linked to nucleotides or nucleotide analogs) may also be quenched (e.g., by incorporation of other nucleotides that may or may not comprise labels). In some cases, as described elsewhere herein, labeling with a quencher can occur after nucleotide or nucleotide analog incorporation. In some cases, the label may be a type that does not self-quench or exhibit proximity quenching. Non-limiting examples of a label type that does not self-quench or exhibit proximity quenching include Bimane derivatives such as Monobromobimane. The term “proximity quenching,” as used herein, generally refers to a phenomenon where one or more dyes near each other may exhibit lower fluorescence as compared to the fluorescence they exhibit individually. In some cases, the dye may be subject to proximity quenching wherein the donor dye and acceptor dye are within 1 nm to 50 nm of each other. Examples of quenchers include, but are not limited to, Black Hole Quencher Dyes (Biosearch Technologies) (e.g., BH1-0, BHQ-1, BHQ-3, and BHQ-10), QSY Dye fluorescent quenchers (Molecular Probes/Invitrogen) (e.g., QSY7, QSY9, QSY21, and QSY35), Dabcyl, Dabsyl, Cy5Q, Cy7Q, Dark Cyanine dyes (GE Healthcare), Dy-Quenchers (Dyomics) (e.g., DYQ-660 and DYQ-661), and ATTO fluorescent quenchers (ATTO-TEC GmbH) (e.g., ATTO 540Q, ATTO 580Q, and ATTO 612Q). Fluorophore donor molecules may be used in conjunction with a quencher. Examples of fluorophore donor molecules that can be used in conjunction with quenchers include, but are not limited to, fluorophores such as Cy3B, Cy3, or Cy5; Dy-Quenchers (Dyomics) (e.g., DYQ-660 and DYQ-661); and ATTO fluorescent quenchers (ATTO-TEC GmbH) (e.g., ATTO 540Q, 580Q, and 612Q).

As used herein, the term “primer” or “primer molecule” generally refers to a polynucleotide which is complementary to a portion of a template nucleic acid molecule. For example, a primer may be complementary to a portion of a strand of a template nucleic acid molecule. The primer may be a strand of nucleic acid that serves as a starting point for nucleic acid synthesis, such as a primer extension reaction which may be a component of a nucleic acid reaction (e.g., nucleic acid amplification reaction such as PCR). A primer may hybridize to a template strand and nucleotides (e.g., canonical nucleotides or nucleotide analogs) may then be added to the end(s) of a primer, sometimes with the aid of a polymerizing enzyme such as a polymerase. Thus, during replication of a DNA sample, an enzyme that catalyzes replication may start replication at the 3′-end of a primer attached to the DNA sample and copy the opposite strand. A primer (e.g., oligonucleotide) may have one or more functional groups that may be used to couple the primer to a support or carrier, such as a bead or particle. In some embodiments, the primer is an oligonucleotide. In some embodiments, a primer comprises at least 5, 8, 10, 12, 14, 15, 16, 18, 20, 21, 22, 23, 24, or 25 bases. Each possibility represents a separate embodiment of the invention. In some embodiments, a primer comprises at most 15, 20, 25, 30, 35, 40, 45, 50, 60, 70, 75, 80, 90 or 100 bases. Each possibility represents a separate embodiment of the invention.

A primer may be completely or partially complementary to a template nucleic acid. A primer may exhibit sequence identity or homology or complementarity to the template nucleic acid. The homology or sequence identity or complementarity between the primer and a template nucleic acid may be based on the length of the primer. For example, if the primer length is about 20 nucleic acids, it may contain 10 or more contiguous nucleic acid bases complementary to the template nucleic acid. In some embodiments, a primer comprises a non-complementary region. In some embodiments, the non-complementary region is a 5′ region. In some embodiments, the non-complementary region is at most 10, 15, 20, 25, 30, 35, 40, 45, 50, 55, 60, 65, 70 or 75% of the primer. Each possibility represents a separate embodiment of the invention. In some embodiments, the non-complementary region is at most 1, 2, 3, 5, 7, 10, 12, 15, 17, 18, 20, 25, or 30 bases in length. Each possibility represents a separate embodiment of the invention.

The complementarity or homology or sequence identity between the primer and the template nucleic acid may be limited. The length of the primer may be between 8 nucleotide bases to 50 nucleotide bases. The length of the primer may be more than 2 nucleotide bases, more than 3 nucleotide bases, 4 nucleotide bases, 5 nucleotide bases, 6 nucleotide bases, 7 nucleotide bases, 8 nucleotide bases, 9 nucleotide bases, 10 nucleotide bases, I1 nucleotide bases, 12 nucleotide bases, 13 nucleotide bases, 14 nucleotide bases, 15 nucleotide bases, 16 nucleotide bases, 17 nucleotide bases, 18 nucleotide bases, 19 nucleotide bases, 20 nucleotide bases, 21 nucleotide bases, 22 nucleotide bases, 23 nucleotide bases, 24 nucleotide bases, 25 nucleotide bases, 26 nucleotide bases, 27 nucleotide bases, 28 nucleotide bases, 29 nucleotide bases, 30 nucleotide bases, 31 nucleotide bases, 32 nucleotide bases, 33 nucleotide bases, 34 nucleotide bases, 35 nucleotide bases, 37 nucleotide bases, 40 nucleotide bases, 42 nucleotide bases, 45 nucleotide bases, 47 nucleotide bases or 50 nucleotide bases. The length of the primer may be less than 50 nucleotide bases, 47 nucleotide bases, 45 nucleotide bases, 42 nucleotide bases, 40 nucleotide bases, 37 nucleotide bases, 35 nucleotide bases, 34 nucleotide bases, 33 nucleotide bases, 32 nucleotide bases, 31 nucleotide bases, 30 nucleotide bases, 29 nucleotide bases, 28 nucleotide bases, 27 nucleotide bases, 26 nucleotide bases, 25 nucleotide bases, 24 nucleotide bases, 23 nucleotide bases, 22 nucleotide bases, 21 nucleotide bases, 20 nucleotide bases, 19 nucleotide bases, 18 nucleotide bases, 17 nucleotide bases, 16 nucleotide bases, 15 nucleotide bases, 14 nucleotide bases, 13 nucleotide bases, 12 nucleotide bases, 11 nucleotide bases, 10 nucleotide bases, 9 nucleotide bases, 8 nucleotide bases, 7 nucleotide bases, 6 nucleotide bases, 5 nucleotide bases, 4 nucleotide bases, 3 nucleotide bases or 2 nucleotide bases.

The term “% sequence identity” may be used interchangeably herein with the term “% identity” and may refer to the level of nucleotide sequence identity between two or more nucleotide sequences, when aligned using a sequence alignment program. As used herein, 80% identity may be the same thing as 80% sequence identity determined by a defined algorithm and means that a given sequence is at least 80% identical to another length of another sequence. The % identity may be selected from, e.g., at least 60%, at least 65%, at least 70%, at least 75%, at least 80%, at least 85%, at least 90%, at least 95%, or at least 99% or more sequence identity to a given sequence. The % identity may be in the range of, e.g., about 60% to about 70%, about 70% to about 80%, about 80% to about 85%, about 85% to about 90%, about 90% to about 95%, or about 95% to about 99%.

The terms “% sequence homology” or “percent sequence homology” or “percent sequence identity” may be used interchangeably herein with the terms “% homology,” “% sequence identity,” or “% identity” and may refer to the level of nucleotide sequence homology between two or more nucleotide sequences, when aligned using a sequence alignment program. For example, as used herein, 80% homology may be the same thing as 80% sequence homology determined by a defined algorithm, and accordingly a homologue of a given sequence has greater than 80% sequence homology over a length of the given sequence. The % homology may be selected from, e.g., at least 60%, at least 65%, at least 70%, at least 75%, at least 80%, at least 85%, at least 90%, at least 95%, or at least 99% or more sequence homology to a given sequence. The % homology may be in the range of, e.g., about 60% to about 70%, about 70% to about 80%, about 80% to about 85%, about 85% to about 90%, about 90% to about 95%, or about 95% to about 99%.

As used herein, the term “primer extension” generally refers to the binding of a primer to a strand of the template nucleic acid, followed by elongation of the primer(s). It may also include, denaturing of a double-stranded nucleic acid and the binding of a primer strand to either one or both of the denatured template nucleic acid strands, followed by elongation of the primer(s). Primer extension reactions may be used to incorporate nucleotides or nucleotide analogs to a primer in template-directed fashion by using enzymes (e.g., polymerizing enzymes such as polymerases). A primer extension reaction may be a process of a nucleic acid amplification reaction.

The term “adapter” or “adaptor,” as used herein, generally refers to a molecule (e.g., polynucleotide) that is adapted to permit a sequencing instrument to sequence a target polynucleotide, such as by interacting with a target nucleic acid molecule to facilitate sequencing (e.g., next generation sequencing (NGS)). The sequencing adapter may permit the target nucleic acid molecule to be sequenced by the sequencing instrument. For instance, the sequencing adapter may comprise a nucleotide sequence that hybridizes or binds to a capture polynucleotide attached to a solid support of a sequencing system, such as a bead or a flow cell. The sequencing adapter may comprise a nucleotide sequence that hybridizes or binds to a polynucleotide to generate a hairpin loop, which permits the target polynucleotide to be sequenced by a sequencing system. The sequencing adapter may include a sequencer motif, which may be a nucleotide sequence that is complementary to a flow cell sequence of another molecule (e.g., a polynucleotide) and usable by the sequencing system to sequence the target polynucleotide. The sequencer motif may also include a primer sequence for use in sequencing, such as sequencing by synthesis. The sequencer motif may include the sequence(s) for coupling a library adapter to a sequencing system and sequence the target polynucleotide (e.g., a sample nucleic acid).

As described herein, an adapter may have a first sub-part and a second sub-part. The first sub-part and the second sub-part may have sequence complementarity. An adapter as described herein may be a paired-end adapter useful for generating paired-end sequence reads. An adapter may comprise a barcode.

The term “barcode” or “barcode sequence,” as used herein, generally refers to one or more nucleotide sequences that may be used to identify one or more particular nucleic acids (e.g., based on their association with a particular sample, derivation from a particular source such as a particular cell, inclusion in a particular partition or other compartment, etc.). A barcode may comprise at least 1, 2, 3, 4, 5, 6, 7, 8, 9, 10, I1, 12, 13, 14, 15, 16, 17, 18, 19, 20, or more nucleotides (e.g., consecutive nucleotides). Each possibility represents a separate embodiment of the invention. A barcode may comprise at least about 10, about 20, about 30, about 40, about 50, about 60, about 70, about 80, about 90, about 100 or more consecutive nucleotides. Each possibility represents a separate embodiment of the invention. All of the barcodes used for an amplification and/or sequencing process (e.g., NGS) may be different. The diversity of different barcodes in a population of nucleic acids comprising barcodes may be randomly generated or non-randomly generated.

A barcode may be comprised of one or more segments. For example, a barcode may comprise a first segment that has a first nucleic acid sequence and a second segment that has a second nucleic acid sequence. The first nucleic acid sequence may be the same or different than the second nucleic acid sequence. Barcode sequences comprising multiple segments may be assembled in a combinatorial fashion according to a split-pool scheme, in which a plurality of different first segments are distributed amongst a plurality of first partitions, the contents which are then pooled and distributed amongst a plurality of second partitions. A plurality of different second segments are then distributed amongst the plurality of second partitions and linked to the plurality of different first segments within the plurality of second partitions, and then the contents of the plurality of second partitions are pooled. The process may be repeated any number of times using any number of different segments and partitions to provide a desired level of barcode diversity. In some cases, the first segment of a barcode sequence may be coupled to a bead. In some embodiments, the barcode comprises a unique molecular identifier (UMI). In some embodiments, a portion of the barcode is an UMI. In some embodiments, the adapter comprises an UMI.

As described herein, the use of barcodes may permit high-throughput analysis of multiple samples using next generation sequencing techniques. A sample comprising a plurality of nucleic acid molecules may be distributed throughout a plurality of partitions (e.g., droplets in an emulsion), where each partition comprises a nucleic acid barcode molecule comprising a unique barcode sequence. The sample may be partitioned such that all or a majority of the partitions of the plurality of partitions include at least one nucleic acid molecule of the plurality of nucleic acid molecules. A nucleic acid molecule and nucleic acid barcode molecule of a given partition may then be used to generate one or more copies and/or complements of at least a sequence of the nucleic acid molecule (e.g., via nucleic acid amplification reactions), which copies and/or complements comprise the barcode sequence of the nucleic acid barcode molecule or a complement thereof. The contents of the various partitions (e.g., amplification products or derivatives thereof) may then be pooled and subjected to sequencing. In some cases, nucleic acid barcode molecules may be coupled to beads. In such cases, the copies and/or complements may also be coupled to the beads. Nucleic acid barcode molecules, and copies and/or complements may be released from the beads within the partitions or after pooling to facilitate nucleic acid sequencing using a sequencing instrument. Because copies and/or complements of the nucleic acid molecules of the plurality of nucleic acid molecules each include a unique barcode sequence or complement thereof, sequencing reads obtained using a nucleic acid sequencing assay may be associated with the nucleic acid molecule of the plurality of nucleic acid molecules to which they correspond. This method may be applied to nucleic acid molecules included within cells divided amongst a plurality of partitions, and/or nucleic acid molecules deriving from a plurality of different samples.

The present disclosure provides a composition comprising a nucleic acid molecule (e.g., a double-stranded nucleic acid molecule) linked to a support (e.g., a solid support, such as a solid particle), which nucleic acid molecule comprises a cleavable or excisable base. The present disclosure also provides methods of isolating and/or enriching for nucleic acid molecules, as well as methods of sequencing nucleic acid molecules.

### Enrichment

The present disclosure provides a composition comprising a nucleic acid molecule (e.g., a double-stranded nucleic acid molecule) comprising a cleavable or excisable moiety (e.g., as described herein), which nucleic acid molecule may be coupled to a support (e.g., a solid support). For example, the nucleic acid molecule may be a double-stranded nucleic acid molecule, which double-stranded nucleic acid molecule may comprise a cleavable or excisable moiety in a single strand. The nucleic acid molecule may also comprise or be coupled to a capture entity (e.g., as described herein). The nucleic acid molecule may be a deoxyribonucleic acid (DNA) molecule. Alternatively, the nucleic acid molecule may be a ribonucleic acid (RNA) molecule. The nucleic acid molecule may be coupled to a support, such as solid support. For example, the nucleic acid molecule may be coupled to a particle (e.g., bead). For example, the nucleic acid molecule may be coupled to a particle (e.g., bead) coupled to a support.

The nucleic acid molecule may be a double-stranded nucleic acid molecule. Alternatively, the nucleic acid molecule may be a single-stranded nucleic acid molecule. Alternatively, the nucleic acid molecule may comprise a ribonucleic acid (RNA). The nucleic acid molecule may comprise a deoxyribonucleic acid (DNA). For example, the nucleic acid molecule may comprise genomic DNA or complementary DNA (cDNA, such as DNA generated by reverse transcribing an RNA molecule). The nucleic acid molecule may comprise a sequence corresponding to a biological sample (e.g., as described herein), or a complement and/or derivative thereof. The nucleic acid molecule may be a product of a ligation reaction. The nucleic acid molecule may be a product of an amplification reaction, such as PCR (e.g., emulsion PCR (ePCR)).

The nucleic acid molecule (e.g., double-stranded nucleic acid molecule) may be included in a solution. For example, the nucleic acid molecule may be immersed in a solution. The solution may comprise a plurality of nucleic acid molecules, such as a plurality of double-stranded nucleic acid molecules or a plurality of single-stranded nucleic acid molecules. A plurality of nucleic acid molecules of a solution may comprise a common nucleic acid sequence. For example, a plurality of nucleic acid molecules of a solution may comprise a same adapter sequence. Alternatively, or in addition, a plurality of nucleic acid molecules of a solution may comprise a same sequence that may be derived from a sample (e.g., a template sequence). For example, a plurality of nucleic acid molecules of a solution may be a clonal population of nucleic acid molecules.

The solution may comprise one or more reagents, including one or more primers, adapters, enzymes (e.g., ligases, polymerases, transcriptases, reverse transcriptases, glycosylases, endonucleases, etc.), nucleotides (e.g., deoxyribonucleotides or ribonucleotides, such as deoxyribonucleotide triphosphates or ribonucleotide triphosphates), ions (e.g., calcium ions, strontium ions, magnesium ions, etc.), buffers, or other reagents. The solution may comprise a buffer, such as a PCR buffer. The solution may comprise a plurality of partitions. For example, the solution may comprise a plurality of droplets (e.g., aqueous droplets in oil, or oil droplets in aqueous solution), such as a plurality of droplets in an emulsion. Alternatively, the solution may be devoid of partitions. The solution may comprise a plurality of supports, such as a plurality of particles. For example, the solution may comprise a plurality of droplets comprising a plurality of particles. The plurality of particles may comprise a plurality of nucleic acid molecules (e.g., a plurality of double-stranded nucleic acid molecules or a plurality of single-stranded nucleic acid molecules) coupled thereto.

The nucleic acid molecule may be coupled to a support, such as a solid support. The support may comprise a plurality of nucleic acid molecules coupled to the support. For example, the support may comprise a plurality of nucleic acid molecules coupled thereto, where each nucleic acid molecule of the plurality of nucleic acid molecules comprises a common nucleic acid sequence. The common nucleic acid sequence may be a sequence of a primer or adapter. Alternatively, or in addition to, the common nucleic acid sequence may be derived from a sample (e.g., a template sequence). For example, the support may comprise a clonal population of nucleic acid molecules coupled thereto.

The nucleic acid molecule may be initially provided in a solution and then coupled (e.g., immobilized) to a support (e.g., a solid support). For example, the support may comprise an adapter or primer molecule comprising a sequence that is at least partially complementary to the nucleic acid molecule, and the support may be contacted with a solution including the nucleic acid molecule under conditions sufficient to couple the nucleic acid molecule to the adapter or primer of the support. For example, the nucleic acid molecule may hybridize to the adapter or primer of the support. The nucleic acid molecule may be a single-stranded nucleic acid molecule prior to coupling to the adapter or primer of the support. In an example, the nucleic acid molecule is a single-stranded nucleic acid molecule comprising a sequence that is at least partially complementary to the adapter or primer of the support. Following hybridization of the nucleic acid molecule to the adapter or primer, the adapter or primer may undergo a primer extension process to generate a second strand that is at least partially complementary to the single-strand region of the nucleic acid molecule.

Alternatively, the nucleic acid molecule is a double-stranded nucleic acid molecule comprising a single-stranded region. In some embodiments, the single-stranded region is a single-stranded overhang. In some embodiments, the single-stranded region is at an end of the double-stranded molecule. In some embodiments, the overhang is a 5′ overhang. In some embodiments, the overhang is a 3′ overhang. In some embodiments, the single-stranded region (e.g., overhang) comprises a sequence that is at least partially complementary to the adapter or primer of the support. In some embodiments, the adapter or primer is conjugated to the support at a 5′ end and the overhang is a 3′ overhang. Following hybridization of the double-stranded nucleic acid molecule to the adapter or primer, the adapter or primer may undergo a primer extension process to generate a new strand that displaces the strand of the double-stranded molecule that does not comprise the overhang. In some embodiments, the new strand is at least partially complementary to the single-strand region (e.g., overhang) of the nucleic acid molecule. Alternatively, following hybridization a nick filling reaction is performed to link the adapter or primer to strand that does not comprise the single-stranded region. In some embodiments, nick filling is ligation.

An end of a single-stranded nucleic acid molecule, or a single-stranded region, may be coupled to a support (e.g., a solid support, as described herein). The single-stranded nucleic acid molecule may comprise a capture entity and/or a cleavable base at or near another end of the nucleic acid molecule (e.g., as described herein). The double-stranded nucleic acid molecule may comprise a capture entity and/or a cleavable base at or near another end (the end opposite the single-stranded region). Of the nucleic acid molecule (e.g., as described herein). In some embodiments, the capture entity and/or cleavable base are at a double-stranded end of the double-stranded molecule. An end of a single strand of a double-stranded nucleic acid molecule may be attached (e.g., directly attached, such as via a covalent or non-covalent interaction) to a support. A second strand of the double-stranded nucleic acid molecule may be considered to be immobilized to the support via the first strand attached (e.g., directly attached) to the support. Alternatively, ends of both strands of a double-stranded molecule may be directly attached to the support. The support and a capture entity may be coupled to different strands of a double-stranded nucleic acid molecule. For example, a first strand of a double-stranded nucleic acid molecule may be attached to a support (e.g., a solid support) and a second strand of the double-stranded nucleic acid molecule may comprise or be coupled to a capture entity. Alternatively, the support and a capture entity may be coupled to a same strand of a double-stranded nucleic acid molecule. For example, a first strand of a double-stranded nucleic acid molecule may be attached to a support (e.g., a solid support) and the first strand may also comprise or be coupled to a capture entity (e.g., at or near another end of the strand). The support and a cleavable or excisable moiety may be coupled to different strands of a double-stranded nucleic acid molecule. For example, a first strand of a double-stranded nucleic acid molecule may be attached to a support (e.g., a solid support) and a second strand of the double-stranded nucleic acid molecule may comprise a cleavable or excisable moiety. Alternatively, the support and a cleavable or excisable moiety may be coupled to a same strand of a double-stranded nucleic acid molecule. For example, a first strand of a double-stranded nucleic acid molecule may be attached to a support (e.g., a solid support) and the first strand may also comprise a cleavable or excisable moiety (e.g., at or near another end of the strand). A given strand of a double-stranded nucleic acid molecule may comprise a cleavable or excisable moiety and comprise or be coupled to a capture entity. The given strand may also be directly attached to a support (e.g., solid support).

The nucleic acid molecule may be coupled to a support (e.g., a solid support) in any useful manner. An end of a nucleic acid molecule (e.g., a double-stranded nucleic acid molecule) may be coupled to a support. For example, a 3′ end of a nucleic acid molecule (e.g., a double-stranded molecule) may be attached to a solid support. Alternatively, a 5′ end of a nucleic acid molecule (e.g., a double-stranded molecule) may be attached to a solid support. An end of a nucleic acid molecule attached to a solid support may not be a “free” end. An end of a nucleic acid molecule not attached to a solid support may be a “free” (e.g., unattached) end. For example, the 5′ end of a strand of a nucleic acid molecule may be a free end (e.g., where the 3′ end of the same strand, or the 5′ end of a second strand, is attached to a support). Alternatively, the 3′ end may be a free end (e.g., where the 5′ end of the same strand, or the 3′ end of a second strand, is attached to a support). A 5′ end of a strand of a double-stranded nucleic acid molecule may be attached to a solid support and the other 5′ end may be a “free” (e.g., unattached) end. Alternatively, a 3′ end of a strand of double-stranded nucleic acid molecule may be attached to a solid support and the other 3′ end may be a free end. A free end may comprise or be linked to a capture entity (e.g., as described herein). A free end may not be complementary to an end attached to a solid support.

A nucleic acid molecule (e.g., a double-stranded nucleic acid molecule) may be covalently attached to a support. For example, a strand of a nucleic acid molecule may be covalently attached to a support. Alternatively, or in addition to, a nucleic acid molecule (e.g., a double-stranded nucleic acid molecule) may be non-covalently attached to a support. For example, a strand of a nucleic acid molecule may be non-covalently attached to a support. A primer may be attached to a support (e.g., covalently attached) and a strand of a double-stranded nucleic acid molecule may be synthesized using that primer (e.g., using a primer extension process). For example, a single-stranded template nucleic acid molecule hybridized to a primer attached to a support may be used to synthesize a double-stranded nucleic acid molecule. The single-stranded template may comprise a target sequence. The primer may be extended to produce a reverse complement to the single-stranded template. The extension reaction may generate the double-stranded nucleic acid molecule.

In some embodiments, the support comprises a primer or adapter. In some embodiments, the primer or adapter is conjugated at its 5′ end to the support. The primer or adapter may be conjugated by any method known in the art. Common conjugation methods include a Click reaction, a copper-free Click reaction, a streptavidin to biotin conjugation, an EDC conjugation, and a DSC conjugation, etc. In some embodiments, the support comprises a plurality of primers or adapters. In some embodiments, the plurality of primers or adapters comprise substantially the same sequence. In some embodiments, the support comprises a first plurality of primers or adapters and a second plurality of primers or adapters. In some embodiments, the primers or adapters of the first plurality comprise substantially the same sequence. In some embodiments, the primers or adapters of the second plurality comprise substantially the same sequence. In some embodiments, the primers or adapters of the first plurality comprise a different sequence than the primers or adapters of the second plurality. In some embodiments, the first and second pluralities are employed for bridge amplification.

In some embodiments, the primer or adapter conjugated to the support comprises at least one cleavable or excisable base. In some embodiments, the primer or adapter conjugated to the support comprises a plurality of cleavable or excisable bases. In some embodiments, cleavage does not comprise removal of a base but merely cleavage of a bond between bases. For example, RNase H cleaves the phosphodiester backbone between an RNA bases or DNA-RNA bases, but may not fully excise the RNA bases unless multiple cleavages occur. In some embodiments, cleavage between bases produces a nick. In some embodiments, a single cleavable or excisable base when excised produces a nick. In some embodiments, a single cleavable or excisable base when excised produces a gap. For example, USER enzyme removes DNA uracil bases, complexly excising the base from the molecule. In some embodiments, the gap is a gap of a single base. In some embodiments, a single cleavable or excisable base when cleaved produces a nick. In some embodiments, the plurality of cleavable or excisable bases are configured to generate a gap region when the bases are excised. In some embodiments, the nick comprises a free 5′ end. In some embodiments, the gap region comprises a free 5′ end. In some embodiments, the nick comprises a free 3′ end. In some embodiments, the gap region comprises a free 3′ end. In some embodiments, the free 3′ end acts as a primer for addition of nucleotides into the gap region by a polymerizing agent. In some embodiments, the free 3′ end is capable of priming a polymerizing reaction by a polymerase enzyme.

A nucleic acid molecule may comprise or be coupled to a capture entity. A capture entity may be disposed at a free end of a nucleic acid molecule (e.g., an end not attached to a support). A capture entity may be linked to a nucleotide of a nucleic acid strand at a free end of the nucleic acid strand of the nucleic acid molecule. A capture entity may be proximal to a free end of a strand of a nucleic acid molecule. For example, a capture entity may be within 0, 1, 2, 3, 4, 5, 6, 7, 8, 9 or 10 bases of an end of a strand of a nucleic acid molecule. Each possibility represents a separate embodiment of the invention. In some embodiments, proximal is within 0, 1, 2, 3, 4, 5, 6, 7, 8, 9 or 10 bases. Each possibility represents a separate embodiment of the invention. In some embodiments, proximal is within 5 bases. In some embodiments, proximal is within 3 bases. In some embodiments, proximal is within 10 bases. For example, a capture entity may be within 3 bases of an end of a strand of a nucleic acid molecule. For example, a capture entity may be 0 bases proximal to an end of a strand of a nucleic acid molecule (e.g., the capture entity may be coupled to the last base of the strand). For example, a capture entity may be 1 base proximal to an end of a strand of a nucleic acid molecule (e.g., the capture entity may be coupled to the base adjacent to the last base of the strand). The free end comprising or coupled to a capture entity may not be complementary to an end of a nucleic acid strand that is attached to a support (e.g., a solid support). A capture entity may be coupled to a strand of a nucleic acid molecule that is not directly attached to a support (e.g., a solid support). For example, a capture entity may be coupled to the last base of a strand of a double-stranded nucleic acid molecule that is not attached to a support (e.g., a solid support) (e.g., the capture entity may be coupled to the base furthest from the solid support).

As used herein, the term “capture entity” refers to any molecule that can specifically be bound by a capturing entity. The capture entity may comprise, for example, biotin, avidin, a nucleic acid sequence, a magnetic moiety, a charged moiety, click chemistry, or any other useful moiety. The capture entity and capturing entity may make a pair in which each specifically binds the other. The capture entity may comprise biotin and the capturing entity may comprise an avidin, such as streptavidin (e.g., the capture entity and capturing entity may comprise a biotin-avidin pair). Alternatively, the capture entity may comprise an avidin (e.g., streptavidin) and the capturing entity may comprise biotin. The capture entity may comprise a capture sequence (e.g., a nucleic acid sequence) and the capturing entity may comprise a sequence complementary to the capture sequence. The capture entity may comprise a magnetic particle and the capturing entity may comprise a magnetic field system. The capture entity may comprise a charged particle and the capturing entity may comprise an electric field system. Any useful capture entity/capturing entity pairing may be used for the methods and compositions provided herein.

A nucleic acid molecule (e.g., a double-stranded nucleic acid molecule) may comprise a cleavable or excisable moiety. For example, a nucleic acid molecule may comprise a cleavable base. A cleavable or excisable moiety may be included in a same strand of a nucleic acid molecule as a capture entity. For example, a cleavable or excisable moiety (e.g., a cleavable base) may be proximal to a capture entity. A cleavable or excisable moiety (e.g., a cleavable base) may be proximal to a free end of a strand of a double-stranded molecule. For example, a 5′ end of a strand of a double-stranded molecule may be attached to a support (e.g., a solid support) and a cleavable or excisable moiety (e.g., a cleavable base) may be proximal to the free 5′ end of the second strand of the double-stranded nucleic acid molecule. In some embodiments, the cleavable or excisable base comprises the capture entity. In another example, a 5′ end of a strand of a double-stranded molecule may be attached to a support (e.g., a solid support) and a cleavable or excisable moiety (e.g., a cleavable base) may be proximal to the free 3′ end of the same strand of the double-stranded nucleic acid molecule. In another example, a 5′ end of a strand of a double-stranded molecule may be attached to a support (e.g., a solid support) and a cleavable or excisable moiety (e.g., a cleavable base) may be proximal to the free 5′ end of the second strand of the double-stranded nucleic acid molecule. In another example, a 3′ end of a strand of a double-stranded molecule may be attached to a support (e.g., a solid support) and a cleavable or excisable moiety (e.g., a cleavable base) may be proximal to the free 3′ end of the second strand of the double-stranded nucleic acid molecule. In another example, a 3′ end of a strand of a double-stranded molecule may be attached to a support (e.g., a solid support) and a cleavable or excisable moiety (e.g., a cleavable base) may be proximal to the free 5′ end of the same strand of the double-stranded nucleic acid molecule. A cleavable or excisable moiety may be disposed in proximity to a capture entity. For example, a strand of a nucleic acid molecule may comprise a cleavable or excisable moiety and a capture entity in proximity to one another (e.g., within 0-5 bases of one another, such as within at most 5, 4, 3, 2, 1, or no bases). Alternatively, a nucleic acid molecule may comprise a cleavable or excisable moiety and a capture entity in proximity to one another but associated with different strands of the nucleic acid molecule. In some embodiments, the cleavable or excisable moiety is proximal to a free end. In some embodiments, proximal is sufficiently close that excision of the cleavable or excisable base dissociates all of the bases between the cleavable or excisable base and the free end. In some embodiments, the capture entity is between the cleavable or excisable base and the free end. In some embodiments, excision of the cleavable or excisable base dissociates the base comprising the capture entity. In some embodiments, excision of the cleavable or excisable base removes the capture entity from the molecule.

A cleavable or excisable moiety disposed proximal to a free end of a nucleic acid molecule may be used in the methods of enrichment provided herein. The positioning of a cleavable or excisable moiety (e.g., a cleavable base) near an end of a nucleic acid molecule and in proximity to a capture entity may allow for capture via a capturing entity (e.g., to couple the nucleic acid molecule to a support coupled to the capturing entity) and subsequent release of the nucleic acid molecule by cleavage or excision of the cleavable or excisable moiety. Such a system may allow for any number of enrichment or purification processes, which processes may be performed at any time during processing of the nucleic acid molecule (e.g., using multiple different cleavable or excisable moieties). Cleavable or excisable moieties (e.g., cleavable bases) and capture entities may be coupled to nucleic acid molecules via primers at any stage of nucleic acid processing. For example, coupling of a cleavable or excisable moiety and/or a capture entity to a nucleic acid molecule may be done using, e.g., a primer molecule and followed by a purification and/or release process, allowing simple and rapid enrichment. Integration of a cleavable or excisable moiety (e.g., a cleavable base) and capture entity into a nucleic acid molecule (e.g., a double-stranded nucleic acid molecule) coupled to a support (e.g., a bead) may reduce or eliminate the problem of inter-bead hybridization due to repeat sequences. This results in minimal loss of beads, minimal dropout of sequences near repeats, and an easily performable purification method.

As used herein, the term “cleavable or excisable moiety” generally refers to any moiety that may be cleaved and/or excised from a nucleic acid molecule. A cleavable or excisable moiety may be a cleavable base. As used herein, the term “cleavable base” generally refers to any base or analog of a base (e.g., nucleobase) that can be specifically cleaved and removed or excised from a nucleic acid molecule. Examples of cleavable bases include, but are not limited to, uracil, 8-oxoguanine (also referred to as 8-hydroxyguanine, 8-oxo-7,8-dihydroguanine, 7,8-dihydro-8-oxoguanine, and 8oxoG herein), inosine, an RNA base, and 2,6-diamino-4-hydroxy-5-formamidopyrimidine (FapyG). In some embodiments, the uracil is a DNA uracil base. In some embodiments, the RNA base is in a DNA backbone. In some embodiments, the DNA is devoid of RNA bases other than the cleavable or excisable bases. In some embodiments, the DNA backbone is devoid of RNA bases other than the cleavable or excisable bases. Cleavage and/or excision of a cleavable or excisable moiety may be carried out by contacting the cleavable or excisable moiety (e.g., cleavable base) with a cleaving agent. Examples of cleaving agents include, but are not limited to, uracil DNA glycosylase (UDG), apyrimidinic/apuriric endonuclease (APE), endonucleases (e.g., endonuclease VIII (EndoVIII) or V (EndoV)), uracil-specific excision reagent (USER) enzyme, formamidopyrimidine DNA glycosylase (Fpg), 8-oxoguanine glycosylase (OGG1), and RNase (e.g., RNaseH, such as RNaseHII). Photocleavable or photoexcisable moieties may be cleaved or excised using appropriate application of energy, such as by contacting the moiety with UV light. One or more cleaving agents may be used in combination to cleave or excise a cleavable or excisable moiety. In an example, the cleavable base may be an RNA base in a DNA backbone, and the cleaving agent may be RNase (e.g., RNaseH or RNaseHII). In such a case, the nucleic acid molecule may not be an RNA molecule. In such a case the nucleic acid molecule may be devoid of RNA bases other than the cleavable or excisable bases. In some embodiments, the first cleavable or excisable base is the only RNA base, the second cleavable or excisable base is the only RNA base, the third cleavable or excisable base is the only RNA base, or a combination thereof. In another example, the cleavable base may be a uracil base and the cleaving agent may be selected from uracil DNA glycosylase (UDG), apyrimidinic/apurinic endonuclease (APE), Endonuclease VIII and uracil-specific excision reagent (USER) enzyme. For example, the cleaving agent may be UDG. For example, the cleaving agent may be APE. For example, the cleaving agent may be USER. In another example, the cleavable base may be an inosine base and the cleaving agent may be Endonuclease V (Endo V). In another example, the cleavable base may be 2,6-diamino-4-hydroxy-5-formamidopyrimidine (FapyG) base and the cleaving agent may be formanidopyrimidine DNA glycosylase (Fpg). In another example, the cleavable base may be 8-oxo-7,8-dihydroguanine (8oxoG) and the cleaving agent may be 8-oxoguanine glycosylase (OGG1). In another example, the cleavable base may be a photo-cleavable base and the cleaving agent may be light, such as laser light. Application of a cleaving agent may generate a “nick” in a strand of a nucleic acid molecule. Alternatively, or in addition to, another enzyme may be added to generate a nick, or otherwise functionalize a nick. For example, T4 polynucleotide kinase may be added to remove a 3′ phosphate. An enzyme may be used to remove a lesion, such as a 3′ lesion.

It will be understood by a skilled artisan that the cleaving agent is used with a specific cleavable or excisable base. For example, excision of a DNA uracil base can be achieved by use of UDG. APE, Endonuclease VIII or USER. Similarly, an RNase can be used to excise an RNA base from a DNA backbone. Thus, different cleavable or excisable bases will be removed by different cleaving agents. This allows for simultaneous or sequential cleavage of cleavable or excisable bases. If at least two cleavable or excisable bases are cleaved by the same cleaving agent, then the at least two cleavable or excisable bases can be cleaved simultaneously. If a first cleavable base is cleaved by a first cleaving agent and a second cleavable base is cleaved by a second cleaving agent, then sequential cleaving can be done by first adding one cleaving agent and then the other. For example, if a nucleic (A acid molecule comprises a DNA uracil base and an RNA base, USER can be added first to remove the uracil and subsequently RNase can be added to remove the RNA base. Alternatively, the RNase could be added before the USER enzyme. The two cleaving agents could be added at the same step of a method or at different steps. For example, during pre-enrichment a first cleavable or excisable base can be removed to free the target molecule from the capturing agent. Then during sequencing a second cleavable or excisable base can be removed to generate a nick of gap region for initiating sequencing (such as is described herein). Alternatively, a first cleavable or excisable base can be removed to create a single-stranded overhang to allow hybridization to a solid support and during pre-enrichment a second cleavable or excisable base can be removed to free the target molecule from the capturing agent (such as is described herein).

A nucleic acid molecule may include one or more cleavable or excisable moieties (e.g., one or more cleavable bases). Where a nucleic acid molecule includes more than one cleavable or excisable moieties, the cleavable or excisable moieties may be the same as or different than one another. For example, a nucleic acid molecule may comprise a first cleavable or excisable moiety and a second cleavable or excisable moiety, where the first cleavable or excisable moiety is different than the second cleavable or excisable moiety. The first cleavable or excisable moiety and the second cleavable or excisable moiety may be configured to be cleaved by the same cleaving agent or combination of cleaving agents. In another example, a nucleic acid molecule may comprise a first cleavable or excisable moiety and a second cleavable or excisable moiety, where the first cleavable or excisable moiety and the second cleavable or excisable moiety are of a same type.

Where a cleavable or excisable moiety (e.g., a cleavable base) is proximal to an end of a strand of a nucleic acid molecule, cleavage or excision of the moiety may induce one or more other bases to dissociate from the nucleic acid molecule. For example, a cleavable or excisable moiety may be disposed proximal to a free end of a first strand of a nucleic acid molecule (e.g., within 0, 1, 2, 3, 4, or 5 bases of the end of the first strand), and cleavage or excision of the cleavable or excisable moiety may induce one or more bases of the second strand of the nucleic acid molecule to dissociate from the second strand (e.g., one or more bases at or proximal to the end of the second strand). Dissociation may result from instability generated in the cleaved or excised strand in the form of, e.g., a nick, gap, or hole. In some embodiments, the gap is a gap region. If there is sufficient base-pairing with the uncut strand all the bases are likely to stay attached. However, when only a few bases in a row are coupled to bases in another strand, such as near an end of a strand of a double-stranded nucleic acid molecule, this instability may be sufficient to cause these few bases to dissociate. As described elsewhere herein, a capture entity may be coupled to a base at or proximal to an end of a nucleic acid molecule, such as at or proximal to an end of a strand of a double-stranded nucleic acid molecule. Accordingly, cleavage or excision of a cleavable or excisable moiety in a first strand of a nucleic acid molecule may lead to the dissociation of a base in a second strand of the nucleic acid molecule that comprises or is coupled to a capture entity, such that cleavage or excision of the cleavable or excisable moiety causes release of the capture entity from the nucleic acid molecule. The nucleic acid molecule may be coupled to a support (e.g., a solid support) and the capture entity may be coupled via a capturing entity to another support (e.g., another solid support). Accordingly, dissociation of a base comprising or coupled to the capture entity induced by application of a cleaving agent may result in separation of the nucleic acid molecule from the other support.

The present disclosure provides methods for enriching a nucleic acid molecule (e.g., a double-stranded nucleic acid molecule), such as a nucleic acid molecule coupled to a support (e.g., a particle). In an aspect, the present disclosure provides a method for processing a nucleic acid molecule that may comprise enriching a nucleic acid molecule (e.g., a double-stranded nucleic acid molecule), e.g., within a solution. The method may comprise providing a solution comprising a nucleic acid molecule (e.g., a double-stranded nucleic acid molecule) coupled to a support (e.g., a particle, such as a bead). The nucleic acid molecule may comprise a deoxyribonucleic acid. Alternatively, or in addition to, the nucleic acid molecule may comprise a ribonucleic acid. The nucleic acid molecule may be a double-stranded nucleic acid molecule comprising a first strand and a second strand. The first strand of the nucleic acid molecule may comprise a cleavable or excisable moiety (e.g., as described herein). The second strand of the nucleic acid molecule may comprise a cleavable or excisable moiety (e.g., as described herein). The cleavable or excisable moiety may comprise, for example, uracil, 8-oxoguanine (also referred to as 8-hydroxyguanine, 8-oxo-7,8-dihydroguanine, 7,8-dihydro-8-oxoguanine, and 8oxoG herein), inosine, 2,6-diamino-4-hydroxy-5-formamidopyrimidine (FapyG), an RNA base or a photocleavable base. The cleavable or excisable moiety may be at or near (e.g., proximal to) an end of the first strand (e.g., at an end distal to the support). For example, the cleavable or excisable moiety may be or be coupled to the last base of the first strand. Alternatively, the cleavable or excisable moiety may be or be coupled to a base that is 10 or fewer bases from an end of the first strand, such as within 10, 9, 8, 7, 6, 5, 4, 3, or fewer bases. The cleavable or excisable moiety may be configured to be cleaved with a cleaving agent, such as, for example, uracil DNA glycosylase (UDG), apyrimidinic/apurinic endonuclease (APE), endonucleases (e.g., endonuclease VIII (EndoVIII) or V (EndoV)), uracil-specific excision reagent (USER) enzyme, formamidopyrimidine DNA glycosylase (Fpg), 8-oxoguanine glycosylase (OGG1), RNase (e.g., RNaseH, such as RNaseHII), ultraviolet light, or a combination thereof.

The first strand of the nucleic acid molecule may also comprise or be coupled to a capture entity (e.g., as described herein). The capture entity may comprise, for example, a charged particle, a magnetic particle, a nucleic acid sequence, biotin, avidin, another moiety, or a combination thereof. The capture entity may be coupled to the cleavable or excisable moiety (e.g., cleavable base). Alternatively, the capture entity may be coupled to another base of the first strand. For example, the cleavable or excisable moiety may be or be coupled to a base proximal to an end of the first strand and the capture entity may be coupled to a base closer to the end of the first strand than the cleavable or excisable moiety. The nucleic acid molecule may be included in a solution, such as an aqueous solution. The solution may comprise one or more reagents (e.g., as described herein), such as one or more reagents for cleaving or excising the cleavable or excisable moiety, performing a primer hybridization or extension process, or performing a nucleic acid amplification reaction.

The method may comprise bringing a solution comprising the nucleic acid molecule coupled to the support into contact with a capturing entity. Bringing the solution comprising the nucleic acid molecule coupled to the support into contact with the capturing entity may have the effect of coupling the capture entity and the capturing entity. In some embodiments, the bringing is contacting the capture entity to the capturing entity. In some embodiments, the bringing or contacting is under conditions sufficient for binding of the capture entity to the capturing entity. The capturing entity may comprise, for example, biotin, avidin (e.g., streptavidin), a magnetic field system, an electric field system, a nucleic acid sequence, or a combination thereof. For example, the capture entity may be a magnetic particle and the capturing entity may comprise a magnetic field system. In another example, the capture entity may comprise a biotin moiety and the capturing entity may comprise an avidin moiety (e.g., streptavidin). In another example, the capture entity may comprise a nucleic acid sequence and the capturing entity may comprise a nucleic acid sequence comprising a sequence complementary to that of the capture entity. The capturing entity may be coupled to an additional support (e.g., via a covalent or non-covalent interaction). The additional support may be a particle, such as a bead. The additional support may be a surface, such as a surface of a flow cell, disk, or other component of a sequencing instrument. In an example, the support is a first particle and the additional support is a second particle. Where the capturing entity is coupled to an additional support, coupling the capture entity and the capturing entity may effectively couple the nucleic acid molecule to the additional support. Accordingly, the present disclosure provides a method for preparing a nucleic acid molecule coupled to a first support and a second support, where the first support and the second support may be of a same or different type.

The method may comprise separating a nucleic acid molecule coupled to a capturing entity (e.g., via a capture entity) from other components of a solution. For example, the nucleic acid molecule coupled to the capturing entity may be separated from other nucleic acid molecules in the solution that are coupled to a capturing entity. Such other nucleic acid molecules may include primer molecules or nucleic acid molecules that do not comprise a target nucleic acid sequence of interest, for example. In another example, the nucleic acid molecule coupled to the capturing entity may be separated from supports that are not coupled to a capturing entity. In some embodiments, the supports not coupled to a capturing entity are supports devoid of the target nucleic acid molecule. Separation of the nucleic acid molecule coupled to the capturing entity from other components in the solution may have the effect of enriching the nucleic acid molecule within the solution. Where the nucleic acid molecule comprises a target nucleic acid sequence, such as a target nucleic acid sequence associated with a sample, such separation may have the effect of enriching the target nucleic acid sequence within the solution. In some embodiments, beads comprising the target nucleic acid sequence are enriched within the solution. Where the solution comprises a plurality of such nucleic acid molecules coupled to one or more capturing entities (e.g., a plurality of nucleic acid molecules comprising a target nucleic acid sequence, such as a plurality of nucleic acid molecules coupled to a same support), such separation may have the effect of enriching the target nucleic acid sequence within the solution and may provide a clonal population of nucleic acid molecules of interest with minimal contaminants, which may allow for streamlined and error-reduced downstream processing including nucleic acid sequencing. Where the solution comprises a plurality of supports, some with and some without target nucleic acids bound, separating only supports with target molecules may streamline downstream amplification. Thus, pre-enrichment will allow for only beads with template entering the amplification reaction, reducing reaction waste, increasing coverage, lowering dropout of rare sequences, and improving overall downstream processing including nucleic acid sequencing. Separation of a nucleic acid molecule coupled to a capturing entity from other components of a solution may comprise isolating the nucleic acid molecule coupled to the capturing entity from other materials. Isolation of amplified beads with double-stranded molecules has the added benefit of reducing clumping/aggregation between beads.

A nucleic acid molecule coupled to a capturing entity may be separated from other components in a solution by, for example, washing away other components. For example, the nucleic acid molecule may be coupled to a capturing entity coupled to an additional support, such as a solid surface, and additional materials may be washed away from the solid surface using, e.g., a washing solution, such as a buffered aqueous solution. In an example, the nucleic acid molecule comprises or is coupled to a capture entity that is a magnetic particle, and the capturing entity is a magnetic field system. The nucleic acid molecule comprising or coupled to the magnetic particle may be separated from other materials in a solution via magnetic separation. In another example, the nucleic acid molecule comprises or is coupled to a capture entity that comprises biotin, and the capturing entity comprises avidin (e.g., streptavidin). The nucleic acid molecule comprising or coupled to the biotin moiety may be coupled to an additional support comprising the capturing entity via a biotin-avidin interaction, and subsequently separated from other materials in a solution by washing away materials that are not coupled to the support.

A capture entity of or coupled to a nucleic acid molecule and a capturing entity may be uncoupled. For example, a capture entity and a capturing entity may be uncoupled following separation of the nucleic acid molecule from other materials (e.g., enrichment or isolation of the nucleic acid molecule). Uncoupling of a capture entity and a capturing entity may be accomplished by reversing the conditions of their coupling. For example, where the capture entity comprises a magnetic particle and the capturing entity comprises a magnetic field system, the magnetic field system may be removed or deactivated, effectively decoupling the capture entity and capturing entity (e.g., capturing entity coupled to an additional support, such as a magnetic support). Alternatively, or in addition to, a capture entity of or coupled to a nucleic acid molecule (e.g., an enriched or isolated nucleic acid molecule) may be separated from the nucleic acid molecule, thereby uncoupling the nucleic acid molecule and a capturing entity with which the capture entity may be coupled. Separation of a capture entity from a nucleic acid molecule may comprise cleaving or excising one or more bases of the nucleic acid molecule. For example, the capture entity may be coupled to a cleavable or excisable moiety of the nucleic acid molecule (e.g., a cleavable base), such that cleavage or excision of the cleavable or excisable moiety effectively decouples the nucleic acid molecule and the capture entity. In another example, the capture entity may be coupled to a base in proximity to a cleavable or excisable moiety of the nucleic acid molecule (e.g., a cleavable base), such that cleavage or excision of the cleavable or excisable moiety effectively decouples the nucleic acid molecule and the capture entity (e.g., upon dissociation of the base to which the capture entity is coupled). Cleavage or excision of a cleavable or excisable moiety of a nucleic acid molecule may introduce a nick or gap region (e.g., an absence of one or more bases) in a strand of the nucleic acid molecule. For example, cleavage or excision of a cleavable or excisable moiety may generate a nick. For example, cleavage or excision of a cleavable or excisable moiety may generate a gap region in a strand of the nucleic acid molecule comprising one or more bases, such as at least 1, 2, 3, 4, 5, or more bases. One or more cleavable or excisable moieties of a nucleic acid molecule may be cleaved or excised (e.g., using the same or different cleaving mechanism or agent). For example, two cleavable or excisable moieties, one of which is coupled to or in proximity to a capture entity, in a same strand of a nucleic acid molecule may be cleaved or excised. Cleavage or excision of these moieties may induce generation of a gap region between the two moieties, which gap region may span at least 2, 3, 4, 5, 6, 7, 8, 9, 10, or more bases. Alternatively, or in addition to, the nucleic acid molecule may be conjugated to a support by a first strand and the capture entity may be on a second strand and dissociation of the two strands decouples the support and a single strand of the target nucleic acid molecule from the capturing entity.

Enrichment of a nucleic acid molecule (e.g., as described herein) may comprise isolation of the nucleic acid molecule. Enrichment or isolation of a nucleic acid molecule may be complete or incomplete (e.g., one or more additional materials, such as one or more additional nucleic acid molecules, may not be removed). For example, enrichment may comprise at least a 70, 75, 80, 85, 90, 92, 95, 97, 99 or 100% enrichment of a nucleic acid molecule or population of nucleic acid molecules (e.g., a clonal population). Enrichment may be performed within a solution (e.g., via a magnetic or charge-based separation method). Alternatively, enrichment may comprise transfer to a new buffer or solution, such as upon usage of a washing solution to remove other materials of an initial solution. Enrichment may comprise depletion of single-stranded nucleic acid molecules in favor of double-stranded nucleic acid molecules. Enrichment may comprise depletion of nucleic acid molecules lacking a cleavable or excisable moiety (e.g., a cleavable base), or a cleavable or excisable moiety coupled to a capture entity (e.g., as described herein).

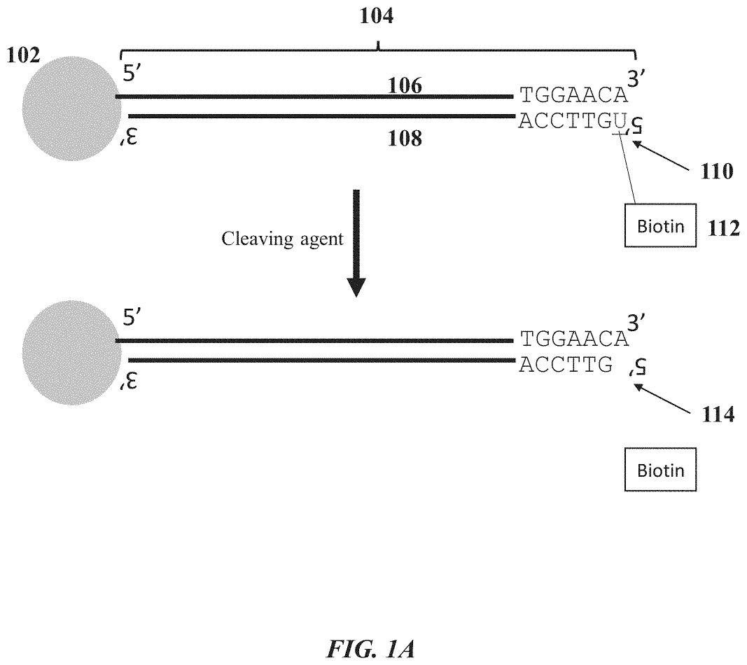

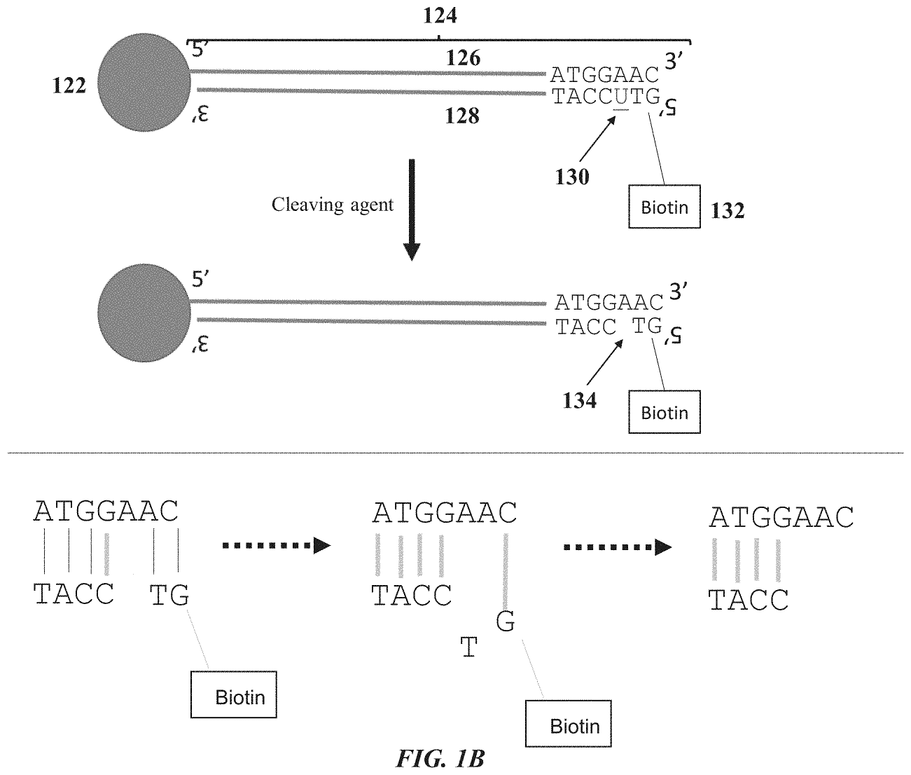

Reference is now made to FIGS. 1A and 1B. FIG. 1A shows a system in which a 5′ end of a double-stranded nucleic acid molecule 104 is coupled to a support 102 (e.g., “solid support” as described herein). Support 102 may be a particle (e.g., a bead). Nucleic acid molecule 104 comprises strands 106 and 108. In some embodiments, strand 106 is a first strand and strand 108 is a second strand. In some embodiments, strand 106 is a second strand and strand 108 is a first strand. In some embodiments, strand 106 is coupled to support 102. In some embodiments, a 5′ end of strand 106 is coupled to solid support 102. In some embodiments, strand 108 is not coupled to solid support 102. In some embodiments, a 3′ end of strand 108 is proximal to support 102 but is not coupled to support 102. Nucleic acid molecule 104 may comprise a cleavable base 110, shown in the figure as a uracil base. In some embodiments, strand 108 comprises the cleavable base 110. Cleavable base 110 is shown at position “zero proximal” to the 5′ end of strand 108 (e.g., it is the terminal base). A capture entity 112, shown as biotin, is coupled to cleavable base 110. It will be understood by a skilled artisan that any capture entity such as is described herein may be employed as capture entity 112. Upon contacting nucleic acid molecule 104 with a cleaving agent (e.g., UDG, APE, etc.), cleavable base 110 is removed, and capture entity (biotin) 112 is released. In some embodiments, the cleaving agent is configured to cleave cleavable base 110. A suitable cleaving agent will be selected based on the cleavable base 110 to be cleaved. Cleavable base 110 is shown as the terminal base of strand 108 and as comprising capture entity 112; however, it will be understood by a skilled artisan that cleavable base 110 need only be sufficiently close to the 5′ end of strand 108 such that excision of cleavable base 110 results in the release of a more 5′ base comprising capture entity 112. In some embodiments, release is dissociation of the base comprising capture entity 112 from strand 106. If, for example, the second base from the 5′ end of strand 108 is cleavable base 110 and the 5′ terminal base of strand 108 comprises capture entity 112, upon excision of cleavable base 110 the terminal base comprising capture entity 112 will dissociate. Such an embodiment is depicted in FIG. 1B. If capture entity (biotin) 112 had been captured by a second support (e.g., a particle, as described herein) comprising a capturing agent such as an avidin (e.g., streptavidin), cleavage or excision of cleavable base 110 would have released the double-stranded nucleic acid molecule coupled to support 102 from the second support. After removal of cleavable base 110, nucleic acid molecule 104 may comprise a nick 114. If cleavable base 110 were disposed one or more bases from the end of strand 108, the nick 114 may be included within strand 108 rather than at an end of strand 108. FIG. 1B shows such an example.

FIG. 1B shows a system similar to that of FIG. 1A including a 5′ end of a double-stranded nucleic acid molecule 124 coupled to a support 122 (e.g., as described herein). Support 122 may be a particle (e.g., a bead). Nucleic acid molecule 124 comprises strands 126 and 128. In some embodiments, strand 126 is a first strand and strand 128 is a second strand. In some embodiments, strand 126 is a second strand and strand 128 is a first strand. In some embodiments, strand 126 is coupled to support 122 (e.g., solid support). In some embodiments, a 5′ end of strand 126 is coupled to solid support 122. In some embodiments, strand 128 is not coupled to solid support 122. In some embodiments, a 3′ end of strand 128 is proximal to support 122 but is not coupled to support 122. Nucleic acid molecule 124 may comprise a cleavable base 130, shown in the figure as a uracil base. In some embodiments, strand 128 comprises the cleavable base 130. Cleavable base 130 is shown at position 2 bases proximal to the 5′ end of strand 128 (e.g., the cleavable base is 2 bases away from the end of the strand/the terminal base of the strand). A capture entity 132, shown as biotin, is coupled to cleavable base 130. It will be understood by a skilled artisan that any capture entity such as is described herein may be employed as capture entity 132. Upon excision of cleavable base 130 (e.g., as described herein), nucleic acid molecule 124 may comprise a nick 134. After removal of cleavable base 130, a region of only two bases is included at the end of strand 128 hybridized to strand 126. This interaction may be unstable, such that these bases may dissociate away from nucleic acid molecule 124 and into solution (see below the gray line). Dissociation of a first base (shown as T) may be followed or occur at the same time as dissociation of the second base (shown as G), which here is coupled to capture entity 132. Therefore, dissociation of these bases induced by removal of cleavable base 130 causes nucleic acid molecule 124 and capture entity (biotin) 132 to be separated. As described above, were there a second support (e.g., particle) coupled to a capturing agent with which capture entity 132 was configured to couple (e.g., avidin), the removal of the cleavable base 130 would uncouple nucleic acid molecule 124 from the second support.

The method may further comprise providing the support coupled to a single-stranded nucleic acid molecule. The single-stranded nucleic acid molecule may comprise all or a portion of the second strand of the nucleic acid molecule. The single-stranded nucleic acid molecule may comprise an end that is not bound to the support (e.g., a “free” end). This end may be a 3′ end. Alternatively, this end may be a 5′ end. The single-stranded nucleic acid molecule may be devoid of cleavable or excisable moieties. A primer molecule may be provided. The primer molecule may comprise a nucleic acid sequence that is complementary to a nucleic acid sequence at or near an end of the single-stranded nucleic acid molecule distal to the support (e.g., a “free” end). The primer molecule may comprise the cleavable or excisable moiety of the nucleic acid molecule and may comprise or be coupled to the capture entity. For example, the capture entity may be coupled to the cleavable or excisable moiety (e.g., cleavable base) or the cleavable or excisable moiety may be proximal and 5′ to a base that comprises the capture entity. The primer molecule may be coupled to a support, such as a particle. The primer molecule may be releasably coupled to the support, and releasable from the support upon application of a stimulus, such as a chemical stimulus. The single-stranded nucleic acid molecule and the primer molecule may be subjected to conditions sufficient to hybridize the primer molecule to the single-stranded nucleic acid molecule. If coupled to a support, the primer molecule may be subjected to conditions sufficient to release the primer molecule from the support to facilitate interaction between the primer molecule and the single-stranded nucleic acid molecule. The primer molecule hybridized to the single-stranded nucleic acid molecule may be subjected to conditions sufficient to extend the primer molecule to generate the first strand of the nucleic acid molecule, or a portion thereof. This process may be repeated for a plurality of single-stranded nucleic acid molecules coupled to the support.

The method may further comprise providing the support coupled to a primer molecule. The primer molecule may be releasably coupled to the support, and releasable from the support upon application of a stimulus, such as a chemical stimulus. In some embodiments, the method further comprises providing a primer molecule. In some embodiments, the method further comprises providing a support capable of capturing the capture moiety. In some embodiments, the method further comprises providing a support comprising a capturing entity. The primer molecule may comprise a first nucleic acid sequence. The primer may comprise at least one cleavable or excisable moiety. The primer may comprise a plurality of cleavable or excisable moieties. The first nucleic acid sequence may comprise at least one cleavable or excisable base. In some embodiments, at least one is a plurality. In some embodiments, at least 1 is at least 2. In some embodiments, at least 1 is at least 3. In some embodiments, a plurality is at least 3. A template nucleic acid molecule comprising a second nucleic acid sequence may be provided, where the second nucleic acid sequence of the template nucleic acid molecule may be complementary to the first nucleic acid sequence of the primer molecule. The template nucleic acid molecule may be coupled to a support, such as a particle. The template nucleic acid molecule may be releasably coupled to the support, and releasable from the support upon application of a stimulus, such as a chemical stimulus. The template nucleic acid molecule may comprise a non-cleavable or excisable moiety that is complementary to the cleavable or excisable moiety of the nucleic acid molecule. The template nucleic acid molecule and the primer molecule may be subjected to conditions sufficient to hybridize the template nucleic acid molecule to the primer molecule. The method may further comprise subjecting the primer molecule hybridized to the template nucleic acid molecule to conditions sufficient to extend the primer molecule to generate the second strand of the nucleic acid molecule, or a portion thereof. The second strand of the nucleic acid molecule hybridized to the template nucleic acid molecule may be subjected to conditions sufficient to separate the template nucleic acid molecule and the second strand. This process may be repeated for a plurality of primer molecules coupled to the support and, optionally, a plurality of template nucleic acid molecules, thereby generating a plurality of second strands coupled to the support.

The method may further comprise providing an additional primer molecule. In some embodiments, the additional primer molecule comprises (A) a third nucleic acid sequence that is complementary to a fourth nucleic acid sequence of the second strand and (B) a cleavable or excisable moiety, or non-cleavable or excisable analog thereof, wherein the additional primer molecule comprises or is coupled to the capture entity. In some embodiments, the additional primer molecule comprises (A) a third nucleic acid sequence that is complementary to a fourth nucleic acid sequence of the first strand and (B) a cleavable or excisable moiety, or non-cleavable or excisable analog thereof, wherein the additional primer molecule comprises or is coupled to the capture entity. The second strand or first strand and the additional primer molecule may be subjected to conditions sufficient to hybridize the additional primer molecule to the second strand or first strand. The additional primer molecule hybridized to the second strand may be subjected to conditions sufficient to extend the additional primer molecule to generate the first strand of the nucleic acid molecule, or a portion thereof. The additional primer molecule hybridized to the first strand may be subjected to conditions sufficient to extend the additional primer molecule to generate the second strand of the nucleic acid molecule, or a portion thereof. In some embodiments, the second strand is a clonal copy of the second strand. In some embodiments, the first strand is a clonal copy of the first strand. The additional primer molecule may be coupled to a particle. For example, the additional primer molecule may be releasably coupled to the particle, and may be released upon application of a stimulus, such as a chemical stimulus. The method may further comprise subjecting the additional primer molecule coupled to the particle to conditions sufficient to release the additional primer molecule from the particle. This process may be repeated for a plurality of additional primer molecules, thereby generating a plurality of first strands.

The solution comprising the nucleic acid molecule coupled to the support may comprise a plurality of such supports. The plurality of supports may be coupled to a plurality of nucleic acid molecules comprising a plurality of capture entities and a plurality of cleavable and excisable moieties. Each nucleic acid molecule of the plurality of nucleic acid molecules may comprise a first strand and a second strand. Each first strand may comprise a cleavable or excisable moiety of the plurality of cleavable or excisable moieties proximal to an end of the first strand. Each second strand may comprise a cleavable or excisable moiety of the plurality of cleavable or excisable moieties proximal to an end of the second strand. Each first strand may also comprise or be coupled to a capture entity of the plurality of capture entities at or proximal to the end of the first strand. The solution may further comprise a plurality of additional supports, which plurality of additional supports may not be coupled to nucleic acid molecules comprising a plurality of capture entities.

The support may be initially included within a partition, such as a well or a droplet of an emulsion. The nucleic acid molecule may be coupled to the support within the partition.

The nucleic acid molecule may comprise an additional cleavable or excisable moiety (e.g., as described herein). The cleavable or excisable moiety may be separated from the additional cleavable or excisable moiety by at least ten bases. Alternatively, the cleavable or excisable moiety may be separated from the additional cleavable or excisable moiety by fewer than ten bases.

The nucleic acid molecule may comprise or be coupled to an additional capture entity. For example, the additional capture entity may be coupled to the additional cleavable or excisable moiety. The method may further comprise bringing the nucleic acid molecule coupled to the support into contact with an additional capturing entity (e.g., as described herein) under conditions sufficient to couple the additional capture entity and the additional capturing entity, thereby coupling the nucleic acid molecule to the additional capturing entity. The additional capturing entity may be coupled to a further support (e.g., particle). The nucleic acid molecule coupled to the additional capturing entity may be separated from other components of a solution comprising the nucleic acid molecule (e.g., as described herein). The nucleic acid molecule coupled to the additional capturing entity may be subjected to conditions sufficient to cleave or excise the additional cleavable or excisable moiety, thereby uncoupling the nucleic acid molecule and the additional capturing entity. This may comprise bringing the nucleic acid molecule in contact with a cleaving agent. The cleaving agent may be selected from the group consisting of uracil DNA glycosylase (UDG), apyrimidinic/apurinic endonuclease (APE), endonucleases (e.g., endonuclease VIII (EndoVIII) or V (EndoV)), uracil-specific excision reagent (USER) enzyme, formamidopyrimidine DNA glycosylase (Fpg), 8-oxoguanine glycosylase (OGG1), RNase (e.g., RNaseH, such as RNaseHII), ultraviolet light, and a combination thereof.

The nucleic acid molecule may be subjected to conditions sufficient to cleave or excise the additional cleavable or excisable moiety, thereby generating a nick or gap region in the first strand of the nucleic acid molecule. In some embodiments, subjecting to conditions sufficient to cleave or excise the addition cleavable or excisable moiety generates a nick or gap region in the second strand of the nucleic acid molecule. The nucleic acid molecule may be subjected to conditions sufficient to cleave or excise the additional cleavable or excisable moiety comprises bringing the nucleic acid molecule in contact with a cleaving agent. The cleaving agent may be selected from the group consisting of uracil DNA glycosylase (UDG), apyrimidinic/apurinic endonuclease (APE), endonucleases (e.g., endonuclease VIII (EndoVIII) or V (EndoV)), uracil-specific excision reagent (USER) enzyme, formamidopyrimidine DNA glycosylase (Fpg), 8-oxoguanine glycosylase (OGG1), RNase (e.g., RNaseH, such as RNaseHII), ultraviolet light, and a combination thereof. Cleavage or excision of the additional cleavable or excisable moiety may generate a gap region of two or more bases in the first strand of the nucleic acid molecule or the second strand of the nuclei acid molecule. The nucleic acid molecule comprising the nick or gap region may be brought into contact with a polymerase enzyme and a labeled nucleotide. The labeled nucleotide may be configured to emit a signal (e.g., a fluorescent signal, such as upon excitation at an appropriate wavelength or range of wavelengths). The labeled nucleotide may comprise a fluorescent dye (e.g., as described herein). The polymerase enzyme may be a strand-displacement polymerase enzyme. The nucleic acid molecule comprising the nick or gap region may be subjected to conditions sufficient to incorporate the labeled nucleotide into the nick or gap region. The nucleic acid molecule comprising the nick or gap region may be subjected to conditions sufficient to incorporate the labeled nucleotide into a newly synthesized strand attached to the free 3′ end of the gap region or nick. A signal or change in signal may be detected from the labeled nucleotide incorporated into the nick or gap region of the nucleic acid molecule or a newly synthesized strand. The signal or change in signal may be indicative of incorporation of the labeled nucleotide into the nick or gap region of the nucleic acid molecule.

In some embodiments, generating a solution comprising a nucleic acid molecule coupled to a support comprises:
a) providing a solution comprising a plurality of first supports (e.g., particles), wherein each support comprises at least one first primer molecule;
b) bringing the solution into contact with at least one template nucleic acid molecule comprising a 3′ region reverse complementary to the first primer molecule and a non-cleavable base analogous to a cleavable base proximal to a 5′ end of the nucleic acid molecule;
c) subjecting the at least one template nucleic acid molecule and the at least one first primer molecule to conditions sufficient to hybridize the at least one first primer molecule to the at least one template nucleic acid molecule;
d) subjecting the at least one first primer molecule hybridized to the at least one template nucleic acid molecule to conditions sufficient to extend the at least one first primer molecule to generate a nucleic acid molecule comprising a nucleic acid sequence that is reverse-complementary to a sequence of at least one template nucleic acid molecule; and
e) subjecting the nucleic acid molecule to conditions sufficient to dissociate the at least one template nucleic acid molecule from the reverse-complementary nucleic acid sequence to produce the solution comprising a first support coupled to at least one first single-stranded nucleic acid molecule.

In some embodiments, generating a solution comprising a nucleic acid molecule coupled to a support comprises:
a) providing a solution comprising a plurality of first supports (e.g., particles), wherein each support comprises at least one first primer molecule;
b) bringing the solution into contact with at least one double-stranded template nucleic acid molecule, wherein the double-stranded template nucleic acid molecule comprises i) a first strand comprising a 3′ single-stranded region that is reverse complementary to the first primer molecule, and a cleavable base proximal to a 5′ end of the first strand and ii) a second strand;
c) subjecting the at least one double-stranded template nucleic acid molecule and the at least one first primer molecule to conditions sufficient to hybridize the at least one first primer molecule to the single-stranded region of the double-stranded template nucleic acid molecule; and
d) subjecting the at least one first primer molecule and the second strand at least one double-stranded template nucleic acid molecule to conditions sufficient to seal the nick between the at least one first primer molecule and the second strand of the at least one double-stranded template nucleic acid molecule, to produce the solution comprising a first support coupled to at least one first double-stranded nucleic acid molecule.

In some embodiments, the at least one double-stranded template nucleic acid molecule is generated by providing a double-stranded template molecule and adhering an L-shaped adapter to both sides of the at least one double-stranded template nucleic acid molecule. As used herein, an “L-shaped adapter” refers to an adapter comprising a double-stranded annealed region comprising complementarity between a first strand and a second strand and wherein the second strand consists essentially of the region of complementarity; and an overhang portion on the first strand of the polynucleotide adapter. In some embodiments, an L-shaped adapter is an adapter provided in International Patent Application PCT/IB2020/060420.

With respect to the L-shaped adapter, in some embodiments, at least one strand is 3′ blocked. As used herein, the term “3′ blocked” refers to a nucleotide that cannot be extended at its 3′ end by a polymerase enzyme. In some embodiments, a 3′ blocked strand comprises a 3′ modification or modified base. In some embodiments, the modification is a blocking modification. In some embodiments, the modified base is a blocked base. In some embodiments, a blocked base is a base to which polymerase cannot link a new base. In some embodiments, linking is polymerizing on a new base. In some embodiments, a blocked base is selected from a monophosphate nucleotide, a dideoxynucleotide and a 3′ hexanediol modified base. In some embodiments, a blocked base is a monophosphate nucleotide. In some embodiments, a blocked base is dideoxynucleotide. In some embodiments, a blocked base is a 3′ hexanediol modified base. In some embodiments, the overhang portion is a 5′-end overhang. In some embodiments, the first nucleotide at the 5′-end of the second strand is a monophosphate nucleotide. In some embodiments, the first nucleotide at the Y-end of the second strand is a monophosphate nucleotide. In some embodiments, the overhang portion is a 5′-end overhang, and the first nucleotide at the 5′-end of the second strand is a monophosphate nucleotide. In some embodiments, the overhang portion is a 5-end overhang, and the first nucleotide at the 3′-end of the second strand is a monophosphate nucleotide. In some embodiments, the overhang portion is a 3′-end overhang. In some embodiments, the first nucleotide from the 3′-end of the second strand is a dideoxynucleotide. In some embodiments, the first nucleotide from the 5′-end of the second strand is a dideoxynucleotide. In some embodiments, the overhang portion is a 3′-end overhang, and the first nucleotide from the 5′-end of the second strand is a dideoxynucleotide. In some embodiments, the overhang portion is a 3′-end overhang, and the first nucleotide from the 3-end of the second strand is a dideoxynucleotide. In some embodiments, the overhang portion is a 5′-end overhang, and the first nucleotide from the 3′-end of the second strand is a dideoxynucleotide. In some embodiments, the overhang portion is a 5-end overhang, and the first nucleotide from the 5′-end of the second strand is a dideoxynucleotide. In some embodiments, the first nucleotide from the 3′-end of the second strand is a 3′ hexanediol modified base. In some embodiments, the first nucleotide from the 5′-end of the second strand is a 3′ hexanediol modified base. In some embodiments, the overhang portion is a 3′-end overhang, and the first nucleotide from the 5-end of the second strand is a 3′ hexanediol modified base. In some embodiments, the overhang portion is a 3′-end overhang, and the first nucleotide from the 3′-end of the second strand is a 3′ hexanediol modified base. In some embodiments, the overhang portion is a 5′-end overhang, and the first nucleotide from the 3′-end of the second strand is a 3′ hexanediol modified base. In some embodiments, the overhang portion is a 5-end overhang, and the first nucleotide from the 5′-end of the second strand is a 3′ hexanediol modified base.

With respect to the L-shaped adapters, in some embodiments, the second strand is un-extendable. The terms “un-extendable”, “non-extendable” or “blocked” are interchangeable and refer to that a polynucleotide cannot be further polymerized by formation of phosphodiester bonds. In some embodiments, polymerization is template dependent or independent. In some embodiments, polymerization is enzyme dependent or independent. An un-extendable polynucleotide which can be used according to the method of the invention can be produced or comprise chemically modified nucleotides according to any method known in the art of molecular biology. In some embodiments, a 3′ hexanediol modified base renders a polynucleotide “un-extendable”. In some embodiments, dideoxynucleotide renders a polynucleotide “un-extendable”. In some embodiments, an un-extendable polynucleotide comprises a dideoxynucleotide. In some embodiments, an un-extendable polynucleotide comprises a 3′ hexanediol modified base. In some embodiments, the chemically modified nucleotides, e.g., a dideoxynucleotide or 3′ hexanediol modified base, is located at the 3′-end of the un-extendable polynucleotide. In some embodiments, the first strand comprises a 5′ overhang and the second strand is 3′ blocked.

The precise nucleotide sequences of the annealed regions are generally not material to the invention and may be selected by the user. In some embodiments, one strand of the annealed region at least comprises “primer-binding” sequences which enable specific annealing of amplification primers when the templates are in use in a solid-phase amplification reaction. In some embodiments, the annealed region of the first strand comprises the primer binding sequence. In some embodiments, the annealed region of the second strand comprises the primer binding sequence. The primer-binding sequences are thus determined by the sequence of the primers to be ultimately used for solid-phase amplification. The sequence of these primers in turn is advantageously selected to avoid or minimize binding of the primers to the target portions of the templates within the library under the conditions of the amplification reaction but is otherwise not particularly limited. By way of example, if the target portions of the templates are derived from human genomic DNA, then the sequences of the primers to be used in solid phase amplification should ideally be selected to minimize non-specific binding to any human genomic sequence. In some embodiments, the primers do not bind to a sequence found in nature. In some embodiments, the primers do not bind to a sequence found in a target cell. In some embodiments, the cell is a mammalian cell. In some embodiments, the mammal is a human.

The precise nucleotide sequence of the adapters is generally not material to the invention and may be selected by the user such that the desired sequence elements are ultimately included in the common sequences of the library of templates derived from the adapters, for example to provide binding sites for particular sets of universal amplification primers and/or sequencing primers. Additional sequence elements may be included, for example to provide binding sites for sequencing primers which will ultimately be used in sequencing of template molecules in the library, or products derived from amplification of the template library, for example on a solid support. The adapters may further include “tag” sequences, which can be used to tag, or mark template molecules derived from a particular source. In some embodiments, the tag is a barcode.

In some embodiments, the annealed region of the first strand, second strand, or both, comprises a barcode. In some embodiments, the barcode is a nucleotide barcode. In some embodiments, the annealed region of the first strand, second strand, or both, comprises a barcode nucleotide sequence. In some embodiments, the annealed region of the first strand, second strand, or both, comprises a portion of a barcode nucleotide sequence. In some embodiments, the annealed region of the first strand, second strand, or both, comprises a sequence complementary to a barcode nucleotide sequence. In some embodiments, the annealed region of the first strand, second strand, or both, comprises a portion of a sequence complementary to a barcode nucleotide sequence. In some embodiments, the first strand comprises a barcode nucleotide sequence, and the barcode nucleotide sequence extends from the annealed portion into the overhang portion. In some embodiments, the second strand comprises a barcode nucleotide sequence. In some embodiments, the second strand comprises a reverse complement of a barcode nucleotide sequence. Barcode sequences are well known in the art and any such barcode may be used. In some embodiments, the barcode is a sequence not expressed in a target cell. In some embodiments, the barcode is a sequence not expressed in the template nucleic acid molecules. In some embodiments, the barcode is a sequence not expressed in nature.

In some embodiments, a portion is at least 25, 30, 40, 50, 60, 70, 75, 80, 90, 95, 97, 99 or 100%. Each possibility represents a separate embodiment of the invention. In some embodiments, a portion is at least 50%. In some embodiments, a portion is at least 70%. In some embodiments, a portion is at least 90%. In some embodiments, a portion is less than 100%.

In one embodiment, the barcode is one or more nucleic acid molecules. In some embodiments, the barcode is a unique molecular identifier (UMI). In some embodiments, the first strand comprises an UMI. In some embodiments, the second strand comprises an UMI. In some embodiments, the second strand comprises a reverse complement of an UMI. In some embodiments, the annealed region comprises an UMI. In some embodiments, the overhang region comprises an UMI. In some embodiments, the overhang region comprises a barcode. In some embodiments, the UMI extends from the annealed region to the overhang region. In some embodiments, the barcode extends from the annealed region to the overhang region. Nucleic acid molecules, such as DNA strands, present an unlimited number of barcoding options. As used throughout the invention “barcode”, and “DNA barcode”, are interchangeable with each other and have the same meaning. The nucleic acid molecule serving as a DNA barcode is a polymer of deoxynucleic acids or ribonucleic acids or both and may be single-stranded or double-stranded, optionally containing synthetic, non-natural, or altered nucleotide bases. In some embodiments, the nucleic acid molecule is labeled, for instance, with biotin, a radiolabel, or a fluorescent label. Barcodes are well known in the art, and any such barcodes may be used for the performance of the invention.

As will be appreciated by a person skilled in the art, incorporation of unique DNA barcodes into the polynucleotide of the invention (e.g., the adapter) which is ligated to a pool or pools of nucleic acid, such as comprising nucleic acid molecules from different sources, allows the identification of individual or particular nucleic acid source without having to individually sorting each nucleic acid source from the pool, while using assays including, but not limited to, microarray systems. PCR, nucleic acid hybridization (including “blotting”) or high throughput sequencing.

In some embodiments, the barcode comprises or consists of a sequence not found in nature. In another embodiment, the barcode comprises or consists of a sequence which is not substantially identical or complementary to a cell's genomic material (such as to prevent non-specific amplification of an endogenous nucleic acid molecule within a cell's genomic material, e.g., preventing false positive amplification results). In some embodiments, the cell is a mammalian cell. In some embodiments, the mammal is a human. In some embodiments, the barcode is not a full genome. In some embodiments, the barcode is not a chromosome. In some embodiments, the barcode does not have equal to or more than 60%, 65%, 70%, 75%, 80%, 85%, 90%, 95%, 97%, 99% or 100% complementarity to a naturally occurring sequence, or any value and range therebetween. Each possibility represents a separate embodiment of the invention. In some embodiments, the barcode comprises less than 60%, 55%, 50%, 45%, 40%, 35%, 30%, 25%, 20%, 15%, 10%, 5%, 3%, or 1% complementarity to a naturally occurring sequence, or any value and range therebetween. Each possibility represents a separate embodiment of the invention.

In some embodiments, a unique barcode is suitable for identifying a specific or particular subpopulation of nucleic acid molecules within a heterogenous pool of different nucleic acid molecules implementing the methods disclosed by the present the invention. Methods for the detection of the presence and identification of a nucleic acid molecule or sequence are known to a skilled artisan and include sequencing and array (e.g., microarray) systems capable of enhancing the presence of multiple barcodes.

In some embodiments, the overhang region comprises a sequence complementary to a 3′ region of a nucleic acid primer. In some embodiments, the first strand annealed region comprises a sequence complementary to a 3′ region of a nucleic acid primer. In some embodiments, the second strand annealed region comprises a sequence complementary to a 3′ region of a nucleic acid primer.

In some embodiments, the overhang region comprises at least one cleavable or excisable moiety. In some embodiments, the overhang region comprises a plurality of cleavable or excisable moieties. In some embodiments, the method comprises dissociating the first and second strands of the double-stranded template coupled to two adapters. In some embodiments, the single strands are contacted with a second primer. In some embodiments, the second primer comprises a cleavable or excisable moiety. In some embodiments, the cleavable or excisable moiety of the primer is a different cleavable or excisable moiety than the cleavable or excisable moiety of the adapter. In some embodiments, the second primer comprises a capture entity. In some embodiments, the method further comprises subjecting the second primer and the single strand to conditions sufficient to anneal the primer to a 3′ end of the single strand. In some embodiments, the method further comprises extending the second primer to produce a new strand complementary to the single strand thereby producing a fully double-stranded template molecule. In some embodiments, the fully double-stranded template molecule comprises a first fully double-stranded adapter at one end and a second fully double-stranded adapter at the other end. In some embodiments, the first and second fully double-stranded adapters are different. In some embodiments, the second primer is the same primer as is used for amplification.

In some embodiments, the method further comprises contacting the fully double-stranded template molecule with conditions sufficient to cleave or excise the cleavable or excisable moieties in the L-shaped adapters. In some embodiments, excision of the cleavable or excisable moieties from the L-shaped adapter produces a 3′ single-stranded (e.g., overhang) region. In some embodiments, the method produces a double-stranded template molecule such as is described herein.

The at least one template nucleic acid molecule may comprise one or more adapters. In some embodiments, the adapters are single-stranded adapters. In some embodiments, the adapters are double-stranded adapters. In some embodiments, the adapters are fully double-stranded adapters. In some embodiments, the adapters are L-shaped adapters. For example, the at least one template nucleic acid molecule may comprise a first adapter that has been ligated or otherwise attached to the template nucleic acid molecule. The first adapter may comprise a nucleic acid sequence that is a reverse complement to that of the at least one primer coupled to the support of the plurality of supports. The first adapter may comprise the single-stranded region. The first adapter may comprise a double-stranded region comprising a first strand comprising the sequence of the single-stranded region and a second strand comprising a sequence complementary to the sequence of the single-stranded region. In some embodiments, the sequence complementary to the sequence of the single-stranded region comprises at least one cleavable or excisable bases. In some embodiments, excision of the at least one cleavable or excisable bases from the second strand of the adapter produces the single-stranded region. In some embodiments, excision of the at least one cleavable or excisable base from the second strand dissociates the sequence complementary to the sequence of the single-stranded region from the first strand of the adapter, thereby producing the single-stranded region. The solution may comprise a plurality of second supports that is devoid of a template nucleic acid molecule. These supports will be left empty with only the primer coupled thereto and no template and will thus remain empty during subsequent processing. A method of pre-enrichment will remove these empty second supports.

The template nucleic acid molecule may comprise a second adapter that has been ligated or otherwise attached to the template nucleic acid molecule. The second adapter may be disposed at an end of the template nucleic acid molecule that is not complementary to the primer molecule. The second adapter may comprise a base analogous to a cleavable base. The second adapter may comprise a cleavable or excisable base. Alternatively, or in addition to, the template nucleic acid molecule may comprise a non-cleavable base analogous to a cleavable base. A non-cleavable analog may be proximal to an end (e.g., a “free” end) of the template nucleic acid molecule or second adapter thereof. The end may be the 5′ end. The non-cleavable analog may be at the proximal position intended for a cleavable base, which cleavable base may be later incorporated into that position. The cleavable or excisable base may be proximal to an end (e.g., a “free” end) of the template nucleic acid molecule or second adapter thereof. The end may be the 5′ end of a first strand of the template nucleic acid molecule or second adapter thereof. In some embodiments, the first strand of the first adapter and the first strand of the second adapter are a single strand. In some embodiments, the second strand of the first adapter and the second strand of the second adapter are a single strand.

Providing the solution may comprise providing a second nucleic acid molecule comprising a region reverse-complementary to an end (e.g., a “free” end) of the at least one first single-stranded molecule. The second nucleic acid molecule may be a primer molecule. The second nucleic acid molecule may be single-stranded. The second nucleic acid molecule may be double-stranded. The second nucleic acid molecule may be an adapter. The reverse-complementary region may be single-stranded. A second region may be double-stranded. The second nucleic acid molecule may comprise a first cleavable or excisable moiety (e.g., cleavable base). The cleavable or excisable moiety may be proximal to an end of the second nucleic acid molecule. The cleavable or excisable moiety may be proximal to the end that may be reverse-complementary to the free end of the first single-stranded molecule. The second nucleic acid molecule may comprise a capture entity (e.g., as described herein). The capture entity may be at or linked to an end (e.g., a “free” end), such as the 5′ end. The cleavable base may be proximal to the capture entity.

Providing the solution may further comprise subjecting the second nucleic acid molecule to conditions sufficient to hybridize the at least one first single-stranded nucleic acid molecule to the second nucleic acid molecule to produce at least one double-stranded nucleic acid molecule attached to a first support. Such conditions may comprise particular annealing temperatures, which temperatures may be discerned based on the nucleotide content and length of the second nucleic acid molecule and in particular its GC content, for example.

The method may further comprises subjecting the second nucleic acid molecule to conditions sufficient to extend from the second nucleic acid molecule a sequence that is reverse-complementary to the first nucleic acid molecule. This extension may produce a fully double-stranded nucleic acid molecule that may be coupled to the first support. Extension may comprise adding free nucleotides, including labeled nucleotides (e.g., as described herein). Extension may comprise the use of a polymerase enzyme (e.g., as described herein). In some cases, only one round of extension may be performed. In some cases, only one extension reaction may be performed. In some cases, a plurality of rounds of extension may be performed. In some embodiments, a few rounds of extension may be performed. In some embodiments, a few is less than 2, 3, 4, 5, 6, 7, 8, 9, 10, 11, 12, 13, 14 or 15 rounds of extension. Each possibility represents a separate embodiment of the invention. In some embodiments, a few is less than 10. In some embodiments, a few is less than 15. In some embodiments, contacting with the second nucleic acid molecule is clonally amplifying the template nucleic acid molecule.

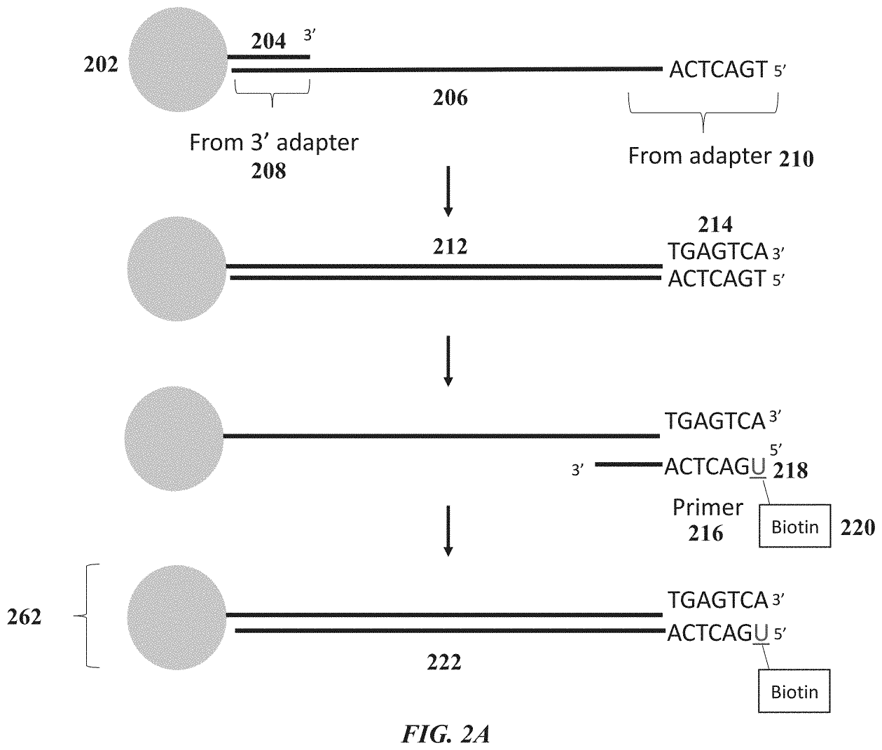

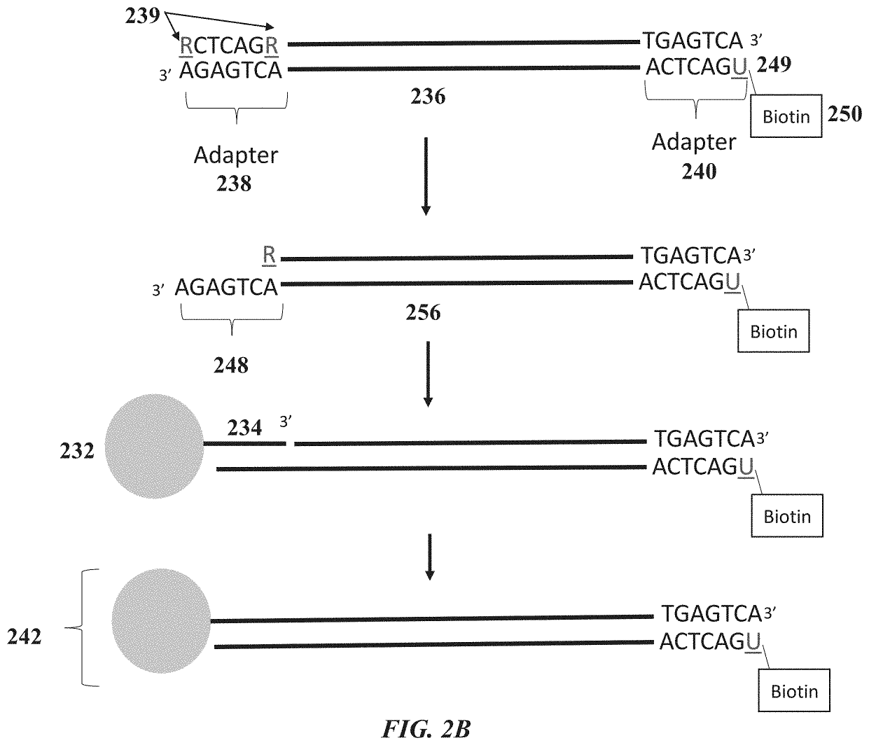

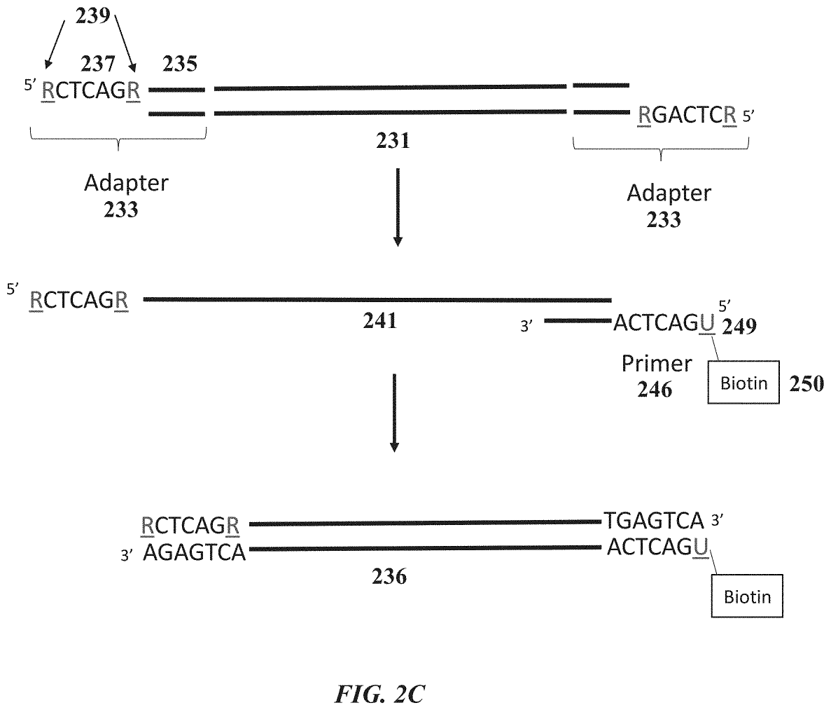

Reference is now made to FIG. 2A, which shows a method for producing a nucleic acid molecule coupled to a support. A support (e.g., bead) 202 may be coupled to primer molecule 204. Primer molecule 204 may comprise at least one cleavable or excisable moiety. A target nucleic acid molecule 206 comprising a 3′ adapter 208 with a nucleic acid sequence complementary to a nucleic acid sequence of primer molecule 204 and a 5′ adapter 210 may be provided. Target nucleic acid molecule 206 may be generated using a library preparation method. Target nucleic acid molecule 206 may be generated using ligation and/or amplification techniques. Adapter sequence 208 of target nucleic acid molecule 206 may be subjected to conditions sufficient to couple (e.g., hybridize) adapter sequence 208 to primer molecule 204, thereby coupling target nucleic acid molecule 206 to support 202. Primer molecule 204 may be subjected to conditions sufficient to perform a primer extension process to extend primer molecule 204 to generate a strand 212 that is complementary to the target nucleic acid molecule, thereby providing a double-stranded nucleic acid molecule coupled to support 202. The double-stranded nucleic acid molecule may comprise a portion 214 having a sequence complementary to that of adapter 210. This double-stranded nucleic acid molecule may not comprise a cleavable or excisable moiety. The double-stranded nucleic acid molecule may, however, comprise a non-cleavable analog to a cleavable or excisable moiety, such as in the second (not coupled to the support) strand. The strand not coupled to the support 202 (e.g., the target nucleic acid molecule 206) may be decoupled from support 202. Primer molecule 216 may be introduced. Primer molecule 216 may comprise a cleavable or excisable moiety 218 (shown here as a uracil base) coupled to a capture entity 220 (shown here as biotin). Primer molecule 216 may comprise a sequence complementary to sequence 214. Primer molecule 216 may be subjected to conditions sufficient to hybridize to sequence 214 and, subsequently, to conditions sufficient to extend primer molecule 216 to generate strand 222. Strand 222 coupled to support 202 may be ligated to support 202 or may not be ligated to support 202; the final product is support-bound duplex target molecule 262. Strand 222 comprising cleavable or excisable moiety 218 and capture entity 220 may undergo an enrichment and/or isolation process, as described herein. Strand 222 may also undergo nucleic acid sequencing, for example after undergoing an enrichment and/or isolation process. Support-bound duplex target molecule 262 comprising cleavable or excisable moiety 218 and capture entity 220 may undergo an enrichment and/or isolation process, as described herein. Support-bound duplex target molecule 262 may also undergo nucleic acid sequencing, for example after undergoing an enrichment and/or isolation process.

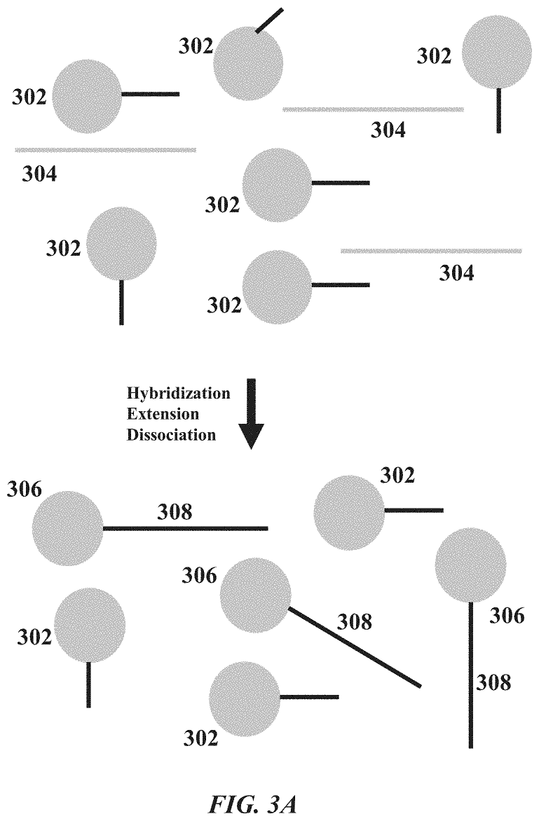

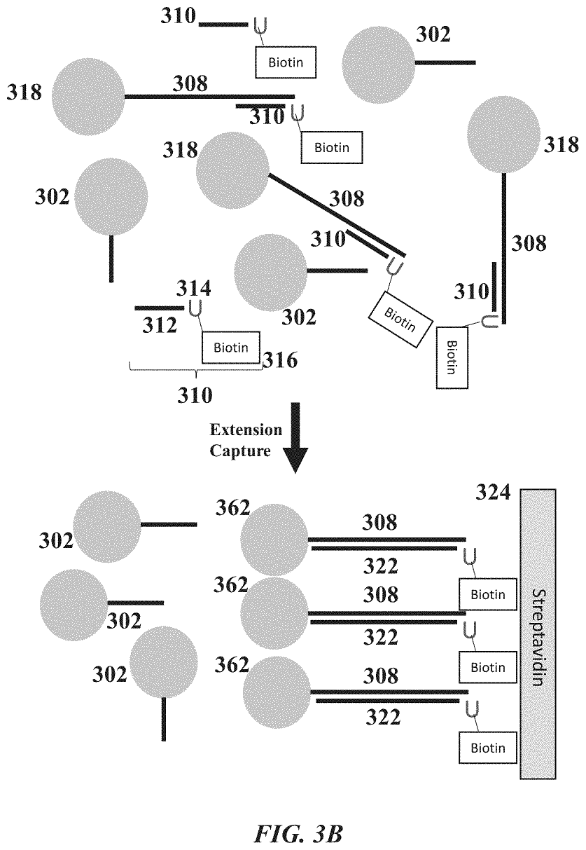

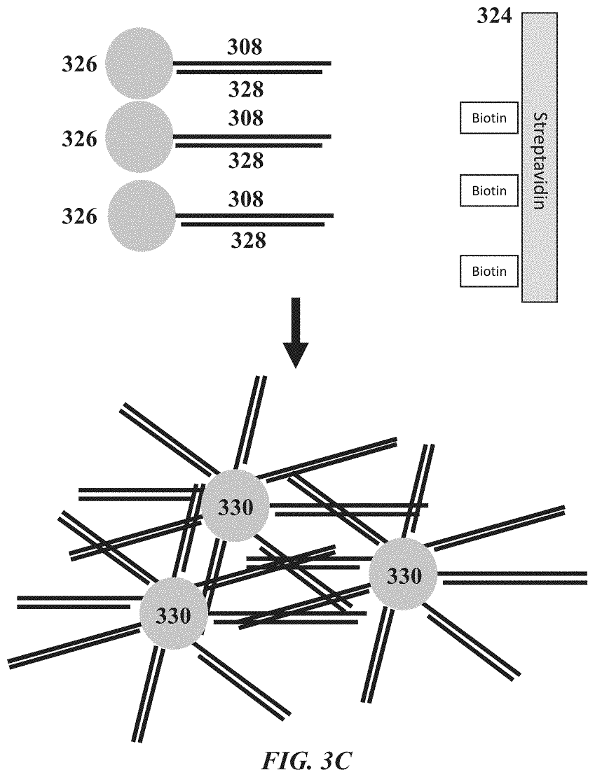

Reference is now made to FIGS. 2B and 2C, which shows an alternative method for producing a nucleic acid molecule coupled to a support. A double-stranded target nucleic acid molecule 236 can be generated according to methods known in the art. Duplex template molecules such as those deriving from a biological sample can be annealed with double-stranded adapters for example or alternatively a method such as is described in PCT/IB2020/060420, herein incorporated by reference in its entirety, may be employed. FIG. 3C shows an exemplary method of producing double-stranded (e.g., duplex) target nucleic acid molecule 236. Double-stranded input template molecule 231 from a biological sample is ligated to L-shaped adapters 233 on both ends. These L-shaped adapters comprise a double-stranded region 235 and a 5′ overhang 237. The 5′ overhang 237 contains at least one cleavable or excisable base 239 and, in the embodiments depicted in FIGS. 2B and 2C, contains two cleavable or excisable bases 239. Cleavable or excisable bases 239 are, in this embodiment, RNA bases within a DNA backbone. This RNA base can be any of an RNA uracil, adenine, guanine, and cytosine. After adapters 238 are ligated on both ends, the resultant duplex molecule is dissociated into two separate single strands 241. These single strands 241 have identical 5′ and 3′ ends and the template region in the middle of one strand is the reverse complement of the template region in the middle of the other. For simplicity only a one single strand 241 is shown. A primer 246 is introduced that contains (i) a 3′ region that is complementary to the 3′ end of single strands 241 and (ii) a 5′ region that is not complementary to any region in either single strand and which comprises another cleavable or excisable moiety 249 wherein the second cleavable or excisable moiety 249 is different than the at least one cleavable or excisable base 239. In this context, different is cleaved by different conditions. As shown in this embodiment, the second cleavable or excisable moiety 249 is a DNA uracil. DNA uracil will not be cleaved by an RNA specific nuclease but can be independently removed from a DNA backbone. The primer also comprises a 5′ capture entity 250 (shown here as biotin). In this example the second cleavable moiety 249 and the capture entity 250 are comprises in the 5′ terminal base; however, it will be understood that an alternative configured in which the cleavable or excisable moiety 249 is proximal to the 5′ end is 3′ to the capture entity 250 is also envisioned. In some embodiments, primer 246 is equivalent or the same as primer molecule 216. Primer 246 is hybridized to single strand 241 and then extended to produce a duplex target nucleic acid molecule 236 (e.g., a duplex template). During extension, the 3′ end of single strand 241 is also extended to produce a region complementary to 5′ end of primer 246. In some embodiments, the 3′ end of the second strand of adapter 233 is blocked and cannot be extended. In such embodiments, the primer is extended to produce a complement to single strand 241, but single strand 241 cannot be extended to include a region that is complementary to the 5′ end of primer 246.

As shown in FIG. 2B, duplex target nucleic acid molecule 236 is provided. Duplex target nucleic acid molecule 236 may be produced by a method such as is shown in FIG. 2C or may be generated by another method known in the art. Duplex target nucleic acid molecule 236 may be generated using a library preparation method. Duplex target nucleic acid molecule 236 may be generated using ligation and/or amplification techniques. Duplex target nucleic acid molecule 236 comprising an adapter 238 at one end and an adapter 240 at the other end. Adapter 238 of duplex target nucleic acid molecule 236 comprises a first strand that comprises at least one cleavable or excisable base 239. In this embodiment, two cleavable or excisable bases 239 are shown and the bases are RNA bases within a DNA backbone. Adapter 240 comprises a first strand comprising another cleavable or excisable moiety 249 and a capture entity 250 (shown here as biotin). Cleavable or excisable bases 239 are configured such that excision of the bases results in the formation of a single-stranded region at a 3′ end of duplex target nucleic acid molecule 236. Cleavable or excisable moiety 249 is configured such that cleavage results in dissociation of capture entity 250 from duplex target nucleic acid molecule 236. In this embodiments, cleavable or excisable moiety 249 is a DNA uracil base. Cleavable or excisable bases 239 and cleavable or excisable moiety 249 are different, such that they are cleaved/excised under different conditions. As cleavable or excisable moiety 249 is a DNA base is will not be cleaved in the presence of an RNA-specific nuclease. Duplex target nucleic acid molecule 236 is now placed in a condition that results in the excision of cleavable or excisable bases 239, but not cleavable or excisable moiety 249. For example, the condition may be addition of an RNA-specific nuclease. Cleavage of cleavable or excisable bases 239 results in the generation of 3′ overhang region 248 within cleaved duplex target molecule 256. A support (e.g., bead) 232 coupled to primer molecule 234 is introduced. Primer molecule 234 is complementary to overhang region 248. Overhang region 248 of cleaved duplex target molecule 256 may be subjected to conditions sufficient to couple (e.g., hybridize) overhang region 248 to primer molecule 234, thereby coupling target nucleic acid molecule 256 to support 232. A gap filling reaction is performed to remove the 5′ ribonucleotide and close the gap region between the 3′ end of primer molecule 234 and the 5′ end of the shorter strand of cleaved duplex molecule 256 to generate support-coupled duplex target 242. Support-coupled duplex target molecule 242 comprises cleavable or excisable moiety 288 and capture entity 250 on a strand that is not directly conjugated to support 232. Support-coupled duplex target molecule 242 is equivalent to support-coupled duplex target molecule 262. Support-coupled duplex target molecule 242 may undergo an enrichment and/or isolation process, as described herein. Support-coupled duplex target molecule 242 may also undergo nucleic acid sequencing, for example after undergoing an enrichment and/or isolation process.

### Pre-Enrichment

The methods provided herein may comprise clonally amplifying a sequence of a nucleic acid molecule (e.g., double-stranded nucleic acid molecule). The clonal population produced may be a double-stranded population. The method may comprise clonally amplifying a sequence of a single-stranded nucleic acid molecule (e.g., the first single-stranded nucleic acid molecule). The clonal population produced may be a single-stranded population. A nucleic acid molecule (e.g., a double-stranded nucleic acid molecule) may be clonally amplified subsequent to undergoing an enrichment or isolation process (e.g., an input population of nucleic acid molecules may be enriched and/or isolated, as provided herein). A nucleic acid molecule (e.g., a double-stranded nucleic acid molecule) may be clonally amplified prior to undergoing an enrichment or isolation process (e.g., a clonal population of nucleic acid molecules may be enriched and/or isolated, as provided herein). Alternatively, or in addition, a nucleic acid molecule (e.g., a single-stranded nucleic acid molecule) may be clonally amplified after undergoing an enrichment or isolation process (e.g., as described herein). Clonal amplification may be performed using a plurality of first primer molecules, which plurality of first primer molecules may be attached to a support (e.g., a particle). A first primer molecule of a plurality of first primer molecules may be a first adapter (e.g., as described herein). Clonal amplification may further comprise the use of a plurality of second primer molecules. A second primer molecule of the plurality of second primer molecules may be homologous to a sequence of the nucleic acid molecule, or a precursor or derivative thereof. A second primer molecule of a plurality of second primer molecules may be a second adapter (e.g., as described herein). A first primer molecule and/or a second primer molecule may comprise one or more cleavable or excisable moieties (e.g., as described herein), which one or more cleavable or excisable moieties may be incorporated into a nucleic acid molecule during an amplification process (e.g., as described herein). A first primer molecule and/or a second primer molecule may comprise or be coupled to a capture entity (e.g., as described herein), which capture entity may be incorporated into or coupled to a nucleic acid molecule during an amplification process (e.g., as described herein). A majority of nucleic acid molecules of a clonal population of nucleic acid molecules may comprise a cleavable or excisable moiety and/or comprise or be coupled to a capture entity. For example, each nucleic acid molecule of a clonal population of nucleic acid molecules may comprise a cleavable or excisable moiety and/or comprise or be coupled to a capture entity. A first primer molecule and/or second primer molecule may comprise a non-cleavable analog of a cleavable or excisable moiety (e.g., cleavable base). Preparation of a clonal population of nucleic acid molecules using such a primer molecule may provide a plurality of nucleic acid molecules comprising the cleavable or excisable moiety.

Amplification of a nucleic acid molecule, including clonal amplification, may be performed within a partition of a plurality of partitions. The partition may be a droplet (e.g., an aqueous droplet) of an emulsion. The partition may be a well (e.g., a microwell of a microwell plate). The partition may comprise a support (e.g., such as a particle, as described herein), which support may be coupled to a nucleic acid molecule or primer molecule (e.g., as described herein). The method may comprise providing a nucleic acid molecule (e.g., a nucleic acid molecule coupled to a support) within a partition. Alternatively, or in addition to, a nucleic acid molecule coupled to a support may be prepared in a bulk solution (e.g., as described herein) and may undergo one or more additional processing steps including, for example, enrichment and/or amplification outside within a partition. Alternatively. or in addition to, a nucleic acid molecule coupled to a support may undergo an amplification process outside of a partition. For example, a nucleic acid molecule coupled to a support may undergo an amplification process in a bulk solution. Alternatively or in addition to, a nucleic acid molecule coupled to a support may be prepared within a partition (e.g., as described herein) and may undergo one or more additional processing steps including, for example, enrichment and/or amplification outside of the partition (e.g., after recovery from the partition). An amplification process (e.g., clonal amplification process) may comprise emulsion PCR (emPCR or ePCR).

Clonal amplification may be performed after performance of an enrichment and/or isolation process described herein. Such an enrichment process may be referred to as a “pre-enrichment” process. A pre-enrichment process may be a pre-PCR enrichment process. A pre-enrichment process may ensure that only complexes comprising a support coupled to a nucleic acid molecule of interest are processed using an amplification process, for example.

Reference is now made to FIGS. 3A-3C, which shows an example of a pre-enrichment process. As shown in FIG. 3A, a plurality of supports (e.g., beads) 302 comprising primer molecules and a plurality of nucleic acid molecules 304 are provided. A support of the plurality of supports may comprise a plurality of primer molecules coupled thereto. The plurality of nucleic acid molecules 304 may comprise target nucleic acid sequences or complements thereof. The plurality of nucleic acid molecules 304 may comprise adapters, such as a first adapter at a first end and a second adapter at a second end. The plurality of nucleic acid molecules 304 may be a plurality of single-stranded nucleic acid molecules. Alternatively, the plurality of nucleic acid molecules 304 may be a plurality of double-stranded nucleic acid molecules. The plurality of nucleic acid molecules 304 may comprise a deoxyribonucleic acid (DNA) and/or a ribonucleic acid (RNA). The supports 302 coupled to primer molecules may be in excess of the plurality of nucleic acid molecules 304 to maximize capture of nucleic acid molecules of the plurality of nucleic acid molecules 304 by supports of the plurality of supports 302. The solution comprising the plurality of supports 302 comprising primer molecules and the plurality of nucleic acid molecules 304 may be subjected to conditions sufficient to hybridize nucleic acid molecules of the plurality of nucleic acid molecules 304 to primer molecules coupled to the plurality of supports 302 and to extend coupled primer molecules to generate a complex 306 comprising a support coupled to a strand 308. Strand 308 may be at least partially complementary to the sequence of a nucleic acid molecule of the plurality of nucleic acid molecules 304. Complex 306 may initially comprise a double-stranded nucleic acid molecule comprising strand 308 and a strand of the plurality of nucleic acid molecules 304 coupled thereto, the strand of the plurality of nucleic acid molecules 304, which may not be directly coupled to the support, may be dissociated to provide complex 306 coupled to strand 308. As the plurality of supports 302 comprising primer molecules were provided in excess, supports of the plurality of supports may remain in solution after the generation of complexes 306. A plurality of primer molecules 310 may be provided, as shown in FIG. 3B. A primer molecule 310 of the plurality of primer molecules may comprise a nucleic acid sequence 312, a cleavable or excisable moiety (shown as a uracil base) 314, and a capture entity (shown as biotin) 316. Nucleic acid sequence 312 of primer molecule 310 may be complementary to a sequence of strand 308 of complex 306. The solution comprising the plurality of complexes 306, supports 302 comprising primer molecules, and the plurality of primer molecules 310 may be subjected to conditions sufficient to hybridize primer molecules 310 to strands 308 to provide complexes 318. A complex 318 may comprise a support coupled to a strand 308 hybridized to a primer molecule 310. The solution may be subjected to conditions sufficient to extend hybridized primer molecules 310 to generate strands 322 having sequences complementary to strands 308. Complexes 362 may each comprise a support coupled to a strand 308 hybridized to a strand 322 comprising a cleavable or excisable moiety 314 and a capture entity 316. Complex 362 is equivalent to support-coupled target duplex molecule 262 and support-coupled target duplex 242. A capturing entity 324, shown in FIG. 3B as a surface comprising a plurality of avidin moieties (streptavidin) coupled thereto, may be introduced. Capture entities 316 may couple to capturing entity 324. Uncomplexed moieties including supports 302 coupled to primer molecules may be separated from captured complexes 362 (e.g., by washing away excess supports 302), thereby enriching and/or isolating complexes 362. A cleaving agent (e.g., UDG, APE, etc.) or combination of cleaving agents may be used to separate complexes 326 from capturing entity 324, as shown in FIG. 3C. A complex 326 may comprise a support coupled to a strand 308 hybridized to a complementary strand 328, which complementary strand may not include the cleavable or excisable moiety 314 and the capture entity 316. Complexes 326 may be enriched within a solution in which they are included (e.g., as described herein). Capturing entity 324 may be removed. Strands 328 may be dissociated from complexes 326. Complexes 326 may be subjected to additional processing including amplification (e.g., clonal amplification, such as amplification by emulsion PCR) to provide clonal supports (e.g., clonal beads) 330 and/or nucleic acid sequencing. The use of double-stranded nucleic acid molecules throughout an enrichment/isolation reduces the risk of bead clumping by hybridization of repeats from one bead to another.

It will be understood by a skilled artisan that the methods shown in FIGS. 2B-2C may be used to generate a support-coupled duplex target molecule 242, which is equivalent to complex 362. Support-coupled duplex target molecule 242 is contacted with capturing entity 324, thereby enriching and/or isolating support-coupled duplex target molecule 242. A cleaving agent (e.g., UDG, APE, etc.) or combination of cleaving agents may be used to separate support-coupled duplex target molecule 242 from capturing entity 324; however, support-coupled duplex target molecule 242 will have lost cleavable or excisable moiety 249 and capture entity 250. Support-coupled duplex target molecule 242 may be enriched within a solution in which they are included (e.g., as described herein). Capturing entity 324 may be removed. Support-coupled duplex target molecule 242 may be subjected to additional processing including amplification (e.g., clonal amplification, such as amplification by emulsion PCR) to provide clonal supports (e.g., clonal beads) 330 and/or nucleic acid sequencing. The use of double-stranded nucleic acid molecules throughout an enrichment/isolation reduces the risk of bead clumping by hybridization of repeats from one bead to another.

### Post-Enrichment

The method may further comprise clonally amplifying a sequence of a nucleic acid molecule (e.g., a double-stranded nucleic acid molecule) using a plurality of first primer molecules attached to a support (e.g., a particle). The clonal amplification may produce a support attached to a clonal population of first nucleic acid molecules. Clonal amplification may be performed prior to an enrichment and/or isolation process described herein. Such an enrichment process may be referred to as a “post-enrichment” process. A post-enrichment process may be a post-PCR enrichment process. Both pre- and post-enrichment processes (e.g., enrichment before and after an amplification process) may be performed. A post-enrichment process may serve to remove “empty” supports (e.g., beads), which may be supports that lack a clonal population of nucleic acid molecules of interest. Both pre- and post-enrichment processes may save reagents and produce a higher percentage of end products of interest.

As described above, a clonal amplification process may be performed with a plurality of primer molecules, which primer molecules may be adapters. An additional plurality of primer molecules (e.g., an additional plurality of adapters) may also be used. Primer molecules and/or additional primer molecules may comprise cleavable or excisable moieties and/or non-cleavable analogs of cleavable or excisable moieties and/or comprise or be coupled to capture entities (e.g., as described herein). Upon incorporation into a nucleic acid molecule coupled to a support, such moieties may be disposed at or near an end of the nucleic acid molecule distal to the support, such as into a 3′ or 5′ end of a strand that is not directly coupled to the support. Primer molecules and/or additional primer molecules may comprise sequences complementary to sequences of a nucleic acid molecule of interest. Additional details and examples of clonal amplification are described elsewhere herein.

A process comprising a post-enrichment process may further comprise contacting a solution comprising a nucleic acid molecule coupled to a first support (e.g., particle, such as a particle comprising a plurality of nucleic acid molecules [e.g., a clonal population of nucleic acid molecules] coupled thereto) with a second support (e.g., particle). The second solid support may comprise or be coupled to a capturing entity (e.g., as described herein). Contacting the solution with the second support may couple the capture entity to the capturing entity, thereby effectively coupling a nucleic acid molecule comprising or coupled to the capture entity to the second support.

A method may further comprise enriching or isolating the second support (e.g., the second support coupled to the nucleic acid molecule). Enrichment or isolating the second support may comprise, for example, using an electric field system (e.g., for a system comprising a charged particle) or a magnetic field system (e.g., for a system comprising a magnetic particle), filtering (e.g., for a system comprising a large particle), centrifugation (e.g., for a system comprising a heavy particle), or a combination thereof. Enrichment or isolation may be less than 100% enrichment or isolation (e.g., as described herein). Such a process may not enrich or isolate a first support that may be not coupled to a second support. Such a process may not enrich or isolate a nucleic acid molecule that is not bound to a second support.

A method may further comprise subjecting a cleavable or excisable moiety of a nucleic acid molecule to conditions sufficient to cleave or excise the cleavable or excisable moiety. Cleaving or excising a cleavable or excisable moiety may release a first support coupled to a clonal population of nucleic acid molecules from a second support to which the first support may be coupled (e.g., via an interaction between a capture entity and capturing entity, as described herein).

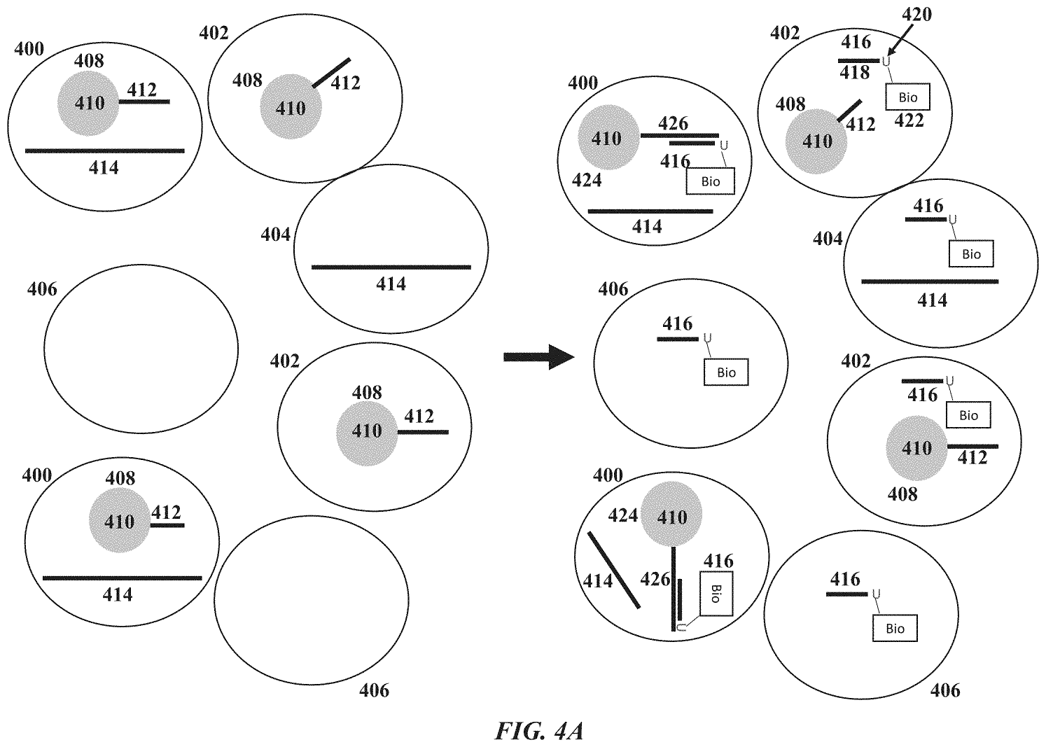

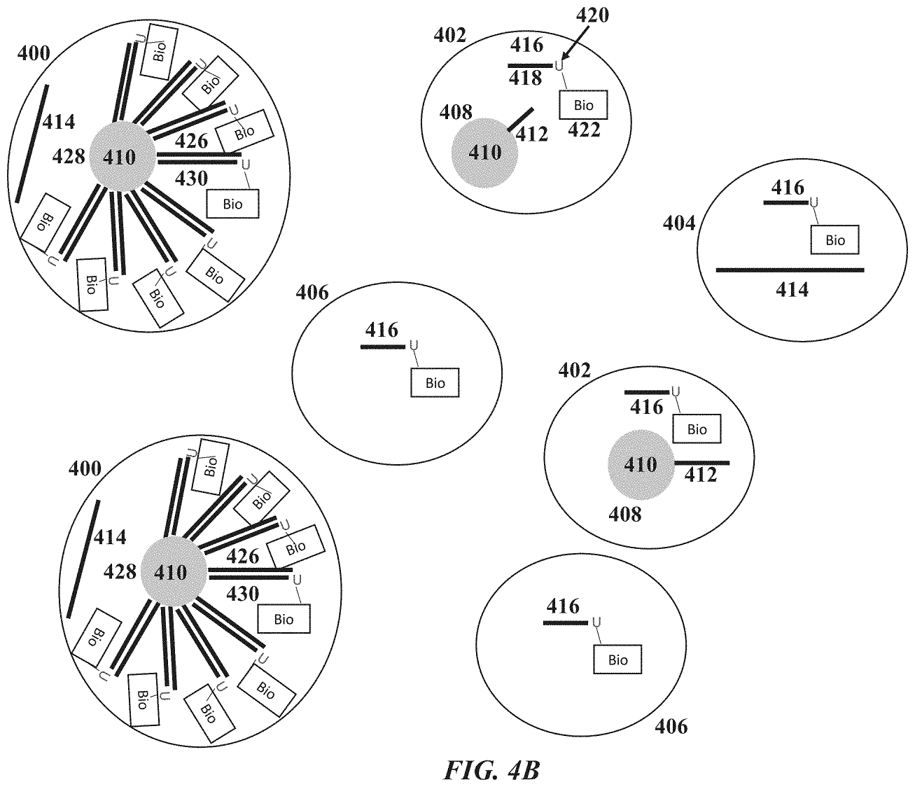

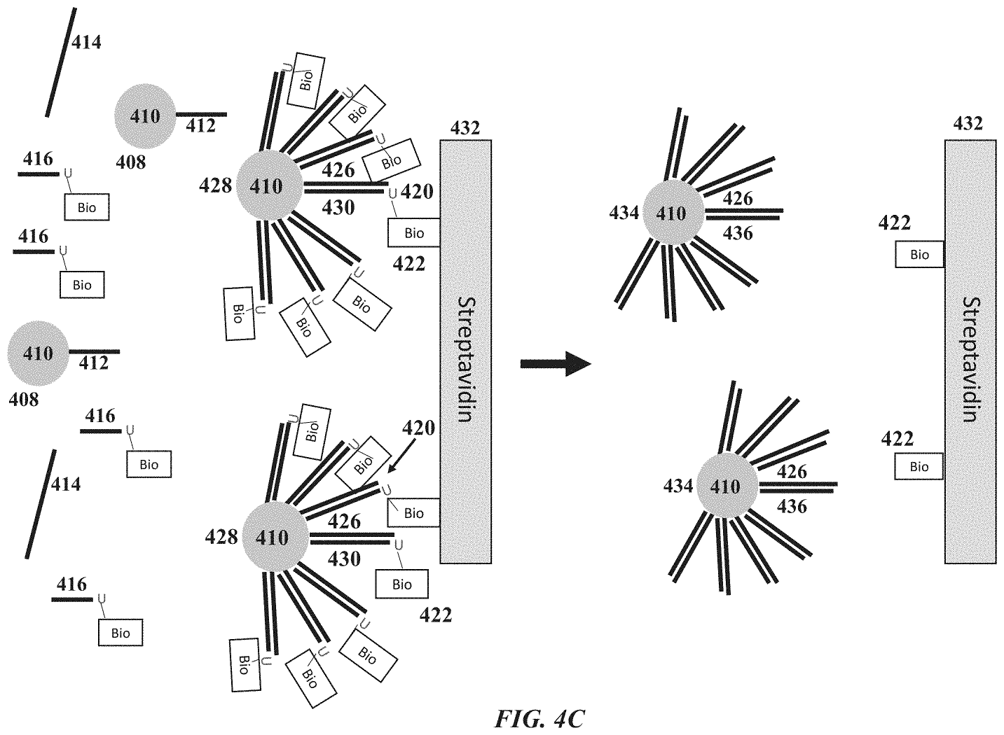

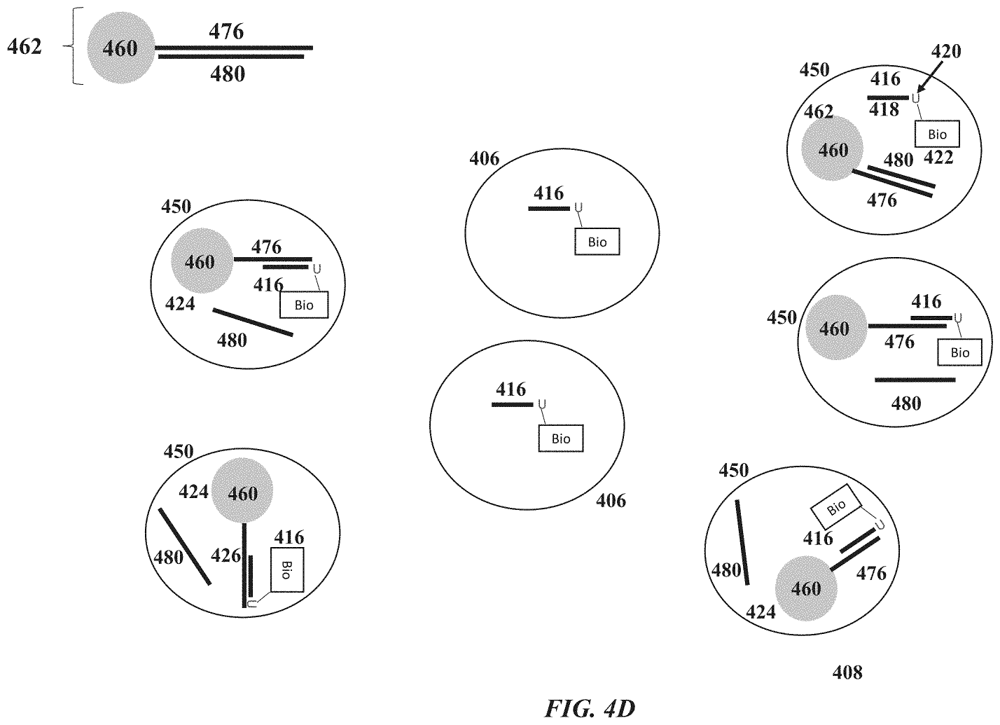

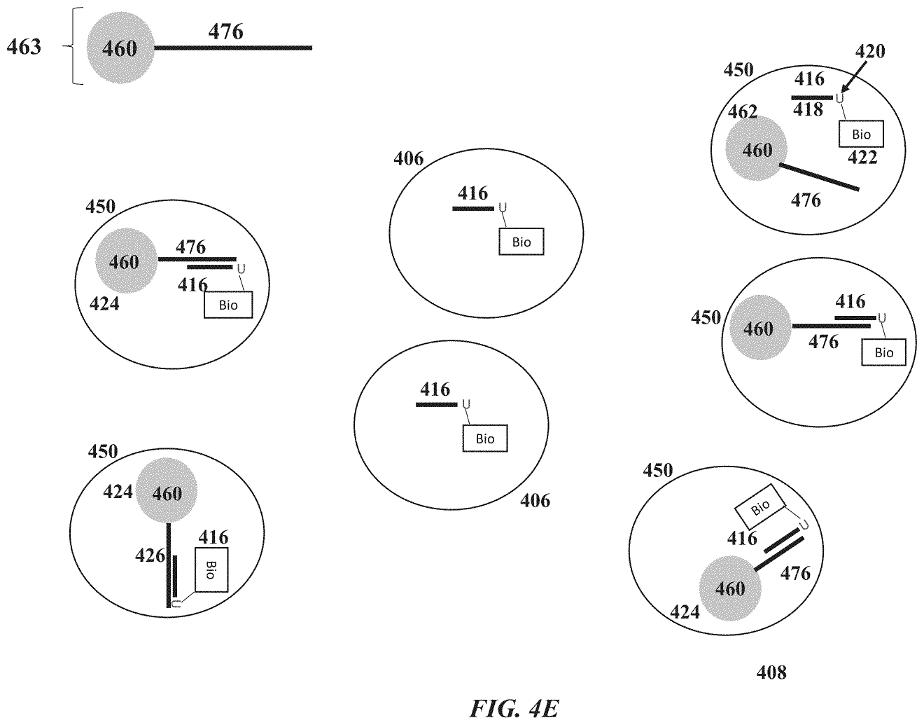

Reference is now made to FIGS. 4A-4C, which show an example of a post-enrichment process. A plurality of partitions (e.g., droplets of an emulsion) are provided. These partitions may be considered to be “microreactors”. Partitions of the plurality of partitions may include one or more different components. For example, partitions 402 may each include a complex 408 comprising a support 410 (e.g., particle) coupled to a primer molecule 412. Partitions 404 may each include a nucleic acid molecule 414 that may comprise a target nucleic acid sequence, or a complementary nucleic acid sequence thereof. A nucleic acid molecule 414 may comprise a sequence that is complementary to all or a portion of a sequence of a primer molecule 412. Partitions 400 may each comprise at least one complex 408 and at least one nucleic acid molecule 414. Partitions 406 may not comprise a complex 408 or a nucleic acid molecule 414 (e.g., such partitions may be empty). Reagents including, for example, enzymes (e.g., polymerase enzymes) and nucleotides may also be included in partitions of the plurality of partitions. As shown in the right panel of FIG. 4A, partitions of the plurality of partitions may also comprise a plurality of primer molecules 416. A primer molecule 416 may comprise a nucleic acid sequence 418, a cleavable or excisable moiety 420 (shown as a uracil base), and a capture entity 422 (shown as a biotin moiety). Nucleic acid sequence 418 may be complementary to a sequence of nucleic acid molecule 414, or a complementary nucleic acid sequence thereof. Nucleic acid sequence 418 may be identical or homologous to a sequence of nucleic acid molecule 414. In a partition 400, complex 408 may be subjected to conditions sufficient to hybridize primer molecule 412 to nucleic acid molecule 414 and to extend primer molecule 412 to generate strand 426 having a sequence complementary to the sequence 418 of nucleic acid molecule 414. Nucleic acid molecule 414 may then be dissociated from strand 426.

As shown in the right panel of FIG. 4A, in a partition 400, nucleic acid sequence 418 of primer molecule 416 and strand 426 coupled to support 410 may be subjected to conditions sufficient to hybridize nucleic acid sequence 418 to a sequence of strand 426. The resultant complex 424 may comprise support 410 coupled to strand 426, which strand is hybridized to primer molecule 416 comprising a cleavable or excisable moiety 420 and a capture entity 422. Complexes 424 cannot be generated in partitions 402, 404, or 406 that did not include complexes 408 and nucleic acid molecules 414.

As shown in FIG. 4B, in a partition 400, a clonal amplification process may be performed to provide a complex 428 comprising a support 410 coupled to a plurality of double-stranded nucleic acid molecules, where a double-stranded nucleic acid molecule of the plurality of double-stranded nucleic acid molecules may comprise a strand 426 and a strand 430 comprising a nucleic acid sequence 418, a cleavable or excisable moiety 420, and a capture entity 422. One or more double-stranded nucleic acid molecules of the plurality of double-stranded nucleic acid molecules may not comprise a cleavable or excisable moiety or a capture entity (not shown). Generation of complex 428 may comprise extension of primer molecule 416 coupled to strand 426, as well as subsequent processing involving one or more additional primer molecules (e.g., as described herein). A given support 410 may comprise a clonal population of such double-stranded nucleic acid molecules, which clonal population may comprise, for example, at least about 100, 1,000, 10,000, 100,000, 500,000, 1,000,000, or more such nucleic acid molecules. The amplification process may comprise emulsion PCR.

As shown in FIG. 4C, contents may be recovered from the plurality of partitions (e.g., an emulsion may be broken). Such contents, including complexes 408, nucleic acid molecules 414, primer molecules 416, and complexes 428 may be provided in a solution, such as a buffered solution. A capturing entity 432 may be provided. The capturing entity 432 may comprise a plurality of avidin (e.g., streptavidin) moieties coupled to a surface. The solution may be subjected to conditions sufficient to couple a capture entity 422 of a complex 428 to capturing entity 432. Complexes 428 coupled to capturing entity 432 may be separated by other components of the solution (e.g., as described herein), such as by washing away the other components including “empty” supports (e.g., as described herein). Complexes 428 may be enriched in a solution and/or isolated (e.g., as described herein). A cleaving agent (e.g., as described herein) may then be used to uncouple capture entities 422 and capturing entity 432, thereby releasing complexes 434 comprising supports 410 coupled to a plurality of double-stranded nucleic acid molecules comprising strands 426 and 436. Such complexes may then be used in downstream processing including nucleic acid sequencing (e.g., as described herein).

Methods of pre-enrichment and post-enrichment may also be combined. As shown in FIG. 4D, a complex 462 is used as the input into a plurality of partitions. Complex 462 is equivalent to complex 326 and is a support-coupled duplex target molecule. As the input automatically has a 1:1 ratio of support (beads) to template (duplex molecules), there is no risk of polyclonally in the beads. This allows a higher ratio of input template to partitions and results in fewer empty partitions and few waste reagents. Partitions of the plurality of partitions may include one or more different components. Reagents including, for example, enzymes (e.g., polymerase enzymes) and nucleotides may also be included in partitions of the plurality of partitions. A plurality of primer molecules 416 are also added to the partitions. Primer molecules 416 may be in such abundance that all partitions receive primers. A primer molecule 416 may comprise a nucleic acid sequence 418, a cleavable or excisable moiety 420 (shown as a uracil base), and a capture entity 422 (shown as a biotin moiety). Nucleic acid sequence 418 may be complementary to a sequence at or near the 3′ end of strand 476. Within partitions 450 containing complex 462, strand 480 which is not coupled to support 460 is dissociated from strand 476 which is coupled. Strand 480 may hybridize to other primers coupled to support 460 or strand 480 may stay in solution within the partition. In a partition 450, complex 462 may be subjected to conditions sufficient to hybridize primer molecule 416 to strand 476 to generate complex 424 At this same time, strand 480 may hybridize to other primers coupled to support 460. Partitions 406 did not receive the complex 462 and contain only primer molecule 416. Amplification and post-enrichment then proceed as detailed in FIGS. 4B and 4C. Alternatively, strand 480 can be dissociated from strand 476 before contact with the partitions. In such an embodiment, complex 463 comprising support 460 and conjugated strand 476 is loaded into the partitions as is shown in FIG. 4E. Once again, the ratio of bead to template molecule is 1:1 ensuring that only clonal amplification occurs. After primer molecule 416 is extended to produce strand 430, strand 430 is made to dissociate and then can hybridize to a free primer on support 460. Amplification and post-enrichment then proceed as detailed in FIGS. 4B and 4C.

### Sequencing

The present disclosure also provides methods of analyzing nucleic acid molecules. A method of analyzing a nucleic acid molecule may comprise nucleic acid sequencing. A method of analyzing a nucleic acid molecule may comprise providing a nucleic acid molecule (e.g., a double-stranded nucleic acid molecule). The nucleic acid molecule may comprise a deoxyribonucleic acid (DNA) and/or a ribonucleic acid (RNA). The nucleic acid molecule may comprise a first strand comprising at least two cleavable or excisable moieties and a second strand. The at least two cleavable or excisable moieties may be independently selected from the group consisting of a ribonucleic acid (RNA) base, a uracil base, an inosine base, 2,6-diamino-4-hydroxy-5-formamidopyrimidine (FapyG) base, 8-oxo-7,8-dihydroguanine (8oxoG) base, and a photocleavable base. Two or more of the at least two cleavable or excisable moieties may be of different types. Alternatively, or in addition, two or more of the at least two cleavable or excisable moieties may be of a same type. In some embodiments, at least two cleavable or excisable moieties is at least three cleavable or excisable moieties. In some embodiments, a moiety is a base.

The nucleic acid molecule may be coupled to a support, such as a particle. The nucleic acid molecule may comprise or be coupled to a capture entity. The capture entity may comprise one or more members selected from the group consisting of biotin, avidin, a nucleic acid sequence, a charged particle, and a magnetic particle. The capture entity may be configured to couple to a capturing entity. The capturing entity may comprise one or more members selected from the group consisting of biotin, avidin, a nucleic acid sequence, an electric field system, and a magnetic field system. The capture entity may be coupled to the capturing entity. The capturing entity may be coupled to an additional support, such as a particle.

The nucleic acid molecule may have undergone an enrichment and/or isolation process (e.g., a pre-enrichment process), as described herein. The nucleic acid molecule (e.g., nucleic acid molecule coupled to a support) may be subjected to conditions sufficient to cleave or excise one or more of the at least two cleavable or excisable moieties, thereby generating a cleaved nucleic acid molecule coupled to the support. Subjecting the nucleic acid molecule to conditions sufficient to cleave or excise one or more of the at least two cleavable or excisable moieties may comprise contacting the nucleic acid molecule with a cleaving agent configured to cleave or excise the one or more cleavable or excisable moieties. The cleaving agent may be selected from the group consisting of, for example, uracil DNA glycosylase (UDG), apyrimidinic/apurinic endonuclease (APE), endonucleases (e.g., endonuclease VIII (EndoVIII) or V (EndoV)), uracil-specific excision reagent (USER) enzyme, formamidopyrimidine DNA glycosylase (Fpg), 8-oxoguanine glycosylase (OGG1), RNase (e.g., RNaseH, such as RNaseHII), ultraviolet light, and a combination thereof. Subjecting the nucleic acid molecule to conditions sufficient to cleave or excise one or more of the at least two cleavable or excisable moieties may decouple the nucleic acid molecule from an additional support where the nucleic acid molecule comprises or is coupled to a capture entity that is coupled to a capturing entity coupled to the additional support.

The cleaved nucleic acid molecule may comprise a nick or gap region. For example, the cleaved nucleic acid molecule may comprise a nick comprising a single base. For example, the cleaved nucleic acid molecule may comprise a gap region comprising two or more bases, such at least 2, 3, 4, 5, 6, 7, 8, 9, 10, or more bases. The nick or gap region may comprise a target nucleic acid sequence, or a complement thereof. Alternatively, or in addition, the nick or gap region may be adjacent to a target nucleic acid sequence, or a complement thereof. In some embodiments, a complement thereof is a complementary nucleic acid sequence thereof.

The method may comprise bringing the cleaved nucleic acid molecule into contact with a polymerase enzyme (e.g., as described herein) and a labeled nucleotide (e.g., as described herein). The labeled nucleotide may comprise a fluorescent dye, such as a fluorescent dye coupled to the nucleotide via a linker. The labeled nucleotide may be configured to emit a signal (e.g., a fluorescent signal). The labeled nucleotide may not be terminated. The labeled nucleotide may be a nucleotide of a plurality of nucleotides. The plurality of nucleotides may comprise a plurality of labeled nucleotides. For example, each nucleotide of the plurality of nucleotides may be a labeled nucleotide. Each nucleotide of the plurality of nucleotides may be of a same type (e.g., comprise a same canonical base, such as A, C, G, and U/T). The cleaved nucleic acid molecule may be subjected to conditions sufficient to incorporate the labeled nucleotide into the nick or gap region of the cleaved nucleic acid molecule. The cleaved nucleic acid molecule may be subjected to conditions sufficient to incorporate the labeled nucleotide into the strand comprising the nick or gap region. The cleaved nucleic acid molecule may be subjected to conditions sufficient to incorporate the labeled nucleotide into a newly synthesized strand. In some embodiments, a newly synthesized strand is a newly synthesizing strand. In some embodiments, the newly synthesized strand is complementary to the strand that does not comprise the nick or gap region. In some embodiments, the strand that does not comprises the nick or gap region is the second strand. In some embodiments, the strand that does not comprise the nick or gap region is the first strand. An additional nucleotide may also be incorporated into the nick or gap region or a position adjacent thereto (i.e., into the newly synthesizing strand). The additional nucleotide may be a labeled nucleotide of the plurality of nucleotides. Where multiple nucleotides are incorporated, such nucleotides may be incorporated into a homopolymeric region of the cleaved nucleic acid molecule. Incorporation of the labeled nucleotide and, optionally, an additional nucleotide may be accomplished using a polymerase enzyme (e.g., as described herein), such as a strand-displacement polymerase enzyme. A signal or change in signal may be detected from the cleaved nucleic acid molecule. The signal or the change in signal may be indicative of incorporation of the labeled nucleotide into the nick or gap region of the cleaved nucleic acid molecule.

The nucleic acid molecule or cleaved nucleic acid molecule may be situated within a partition. The support may be situated within a partition. For example, cleavage or excision of a cleavable or excisable moiety may be performed within a partition. Alternatively, or in addition to, incorporation of a nucleotide (e.g., a labeled nucleotide) may be performed within a partition. A partition may be, for example, a droplet of an emulsion. Alternatively, a partition may be a well.

All or a portion of the sequencing method described above may be repeated one or more times. For example, the support coupled to the cleaved nucleic acid molecule may be brought into contact with an additional plurality of nucleotides (e.g., an additional plurality of nucleotides comprising a plurality of additional labeled nucleotides) and an additional nucleotide of the additional plurality of nucleotides may be incorporated into the cleaved nucleic acid molecule. The additional nucleotide may be of a different type than a previously incorporated labeled nucleotide. The plurality of nucleotides and a nucleotide of the additional plurality of nucleotides may each be nucleobases of different types. The additional nucleotide may be a labeled nucleotide or an unlabeled nucleotide. The additional nucleotide may be non-terminated. Prior to contacting the support with the additional plurality of nucleotides, reagents and nucleotides from the previous sequencing process may be washed away using, e.g., a wash flow. One of more additional solution flows, such as a flow comprising a reagent to remove a label from a labeled nucleotide, or a flow comprising a plurality of unlabeled nucleotides, may also be used. Incorporation of an additional nucleotide into a cleaved nucleic acid molecule may comprise separating a portion of the first strand and a portion of the second strand of the cleaved nucleic acid molecule. Additional signals or changes in signals may be detected after contacting the cleaved nucleic acid molecule with additional nucleotides (e.g., using imaging).

The process described above may be applied to a plurality of nucleic acid molecules, such as a plurality of nucleic acid molecules coupled to a given support or a plurality of nucleic acid molecules coupled to a plurality of supports. All or a portion of a sequencing process may take place within a partition (e.g., as described herein). A method of analyzing a plurality of nucleic acid molecules (e.g., a plurality of deoxyribonucleic acid molecules or a plurality of ribonucleic acid molecules) may comprise providing a support (e.g., particle) comprising the plurality of nucleic acid molecules coupled thereto. The support may be immobilized to another support. For example, the support may be a particle immobilized to a surface. Each nucleic acid molecule of the plurality of nucleic acid molecules may comprise (i) a first strand comprising at least two cleavable or excisable moieties and (ii) a second strand. The at least two cleavable or excisable moieties may be independently selected from the group consisting of a ribonucleic acid (RNA) base, a uracil base, an inosine base, 2,6-diamino-4-hydroxy-5-formamidopyrimidine (FapyG) base, 8-oxo-7,8-dihydroguanine (8oxoG) base, and a photocleavable base. Two or more of the at least two cleavable or excisable moieties may be of different types. Alternatively, or in addition, two or more of the at least two cleavable or excisable moieties may be of a same type. The cleavable or excisable moieties may vary across the plurality of nucleic acid molecules coupled to the support.

The plurality of nucleic acid molecules may comprise or be coupled to a plurality of capture entities. For example, each nucleic acid molecule of the plurality of nucleic acid molecules may comprise or be coupled to a capture entity of the plurality of capture entities. Alternatively, at least one nucleic acid molecule of the plurality of nucleic acid molecules may not comprise or be coupled to a capture entity.

The plurality of nucleic acid molecules may comprise a common nucleic acid sequence. The common nucleic acid sequence may be a target nucleic acid sequence. The plurality of nucleic acid molecules may comprise a clonal population of nucleic acid molecules.

A nucleic acid molecule coupled to the support may have undergone an enrichment and/or isolation process (e.g., a pre-enrichment process), as described herein. The plurality of nucleic acid molecules may have undergone an enrichment and/or isolation process (e.g., a post-enrichment process), as described herein. The support coupled to the plurality of nucleic acid molecules may be subjected to conditions sufficient to cleave or excise one or more of the at least two cleavable or excisable moieties of nucleic acid molecules of the plurality of nucleic acid molecules, thereby generating a plurality of cleaved nucleic acid molecules coupled to the support. Subjecting the support to conditions sufficient to cleave or excise one or more of the at least two cleavable or excisable moieties of nucleic acid molecules of the plurality of nucleic acid molecules may comprise contacting the support with a cleaving agent or a plurality of cleaving agents configured to cleave or excise the one or more cleavable or excisable moieties. Subjecting the support to conditions sufficient to cleave or excise one or more of the at least two cleavable or excisable moieties may decouple one or more nucleic acid molecules from an additional support (e.g., particle) where the one or more nucleic acid molecules comprise or are coupled to a capture entity that is coupled to a capturing entity coupled to the additional support.

The present disclosure may provide a method of sequencing a target nucleic acid sequence. The method may comprise providing a solution comprising a double-stranded nucleic acid molecule, wherein the double-stranded nucleic acid molecule may comprise a target sequence and at least two cleavable or excisable moieties (e.g., cleavable bases) on a first strand; cleaving the at least two cleavable or excisable moieties from the double-stranded nucleic acid molecule to produce a nicked double-stranded nucleic acid molecule; contacting the nicked double-stranded nucleic acid molecule with a polymerase enzyme and labeled nucleotides; and measuring integration of the labeled nucleotides into a newly synthesized nucleic acid molecule complementary or homologous to the target nucleic acid molecule, thereby sequencing a target nucleic acid sequence.

The present disclosure may provide a method of sequencing a double-stranded nucleic acid molecule. The method may comprise isolating at least one support (e.g., particle) attached to a clonal population of double-stranded nucleic acid molecules (e.g., as described herein); contacting the population of double-stranded nucleic acid molecules with a polymerase enzyme and labeled nucleotides; and measuring integration of the labeled nucleotides into a newly synthesized nucleic acid molecule complementary or homologous to a double-stranded nucleic acid molecule of the population of double-stranded nucleic acid molecules, thereby sequencing a double-stranded nucleic acid molecule.

The present disclosure may provide a method of determining a target nucleic acid sequence. The method may comprise providing a double-stranded nucleic acid molecule comprising a first strand comprising the target sequence and a nick 5′ to the target sequence; contacting the nicked double-stranded nucleic acid molecule with a polymerase enzyme and labeled nucleotides; and measuring integration of the labeled nucleotides into a newly synthesized nucleic acid molecule complementary or homologous to the target nucleic acid molecule, thereby determining a target nucleic acid sequence.

The nucleic acid molecule (e.g., double-stranded nucleic acid molecule) may be a DNA molecule. The double-stranded nucleic acid molecule may be coupled to a first support (e.g., first solid support, such as a particle). The double-stranded nucleic acid molecule attached to a first support may be a composition as described herein and may be prepared as described herein. The double-stranded nucleic acid molecule may comprise at least two cleavable or excisable moieties (e.g., cleavable bases) on a first strand. The at least two cleavable or excisable moieties (e.g., cleavable bases) may be a plurality of cleavable or excisable moieties.

The double-stranded nucleic acid molecule may comprise a capture entity (e.g., as described herein) disposed at or near an end of the double-stranded nucleic acid molecule (e.g., an end distal to the support, such as a “free” end). The end may be a 5′ end. The double-stranded nucleic acid molecule may be linked to a second support (e.g., second solid support) comprising a capturing entity (e.g., as described herein) via the capture entity. In such instances, cleaving or excising a cleavable or excisable moiety may release the nicked double-stranded nucleic acid molecule for the second support. The cleaving may release the nicked double-stranded nucleic acid molecule linked to a first support from the second support. This may allow for simultaneous enrichment and nicking of the target molecule, e.g., for sequencing. Thus, a single process can be employed both for purification and beginning a sequencing process which may streamline nucleic acid analysis and allow for isolating and sequencing of a double-stranded template all at once.

The support may comprise a clonal population of double-stranded nucleic acid molecules. Each double-stranded nucleic acid molecule may comprise a first strand comprising the target sequence and a nick 5′ to the target sequence. The nick may be a gap region, such as a gap region of at least 2, 3, 4, 5, 6, 7, 8, 9, 10, or more bases.

The at least two cleavable or excisable moieties may be sufficiently downstream from an end such that the intervening bases may not dissociate from the other strand. The at least two cleavable or excisable moieties may be at a sufficient distance from the end such that excision does not dissociate intervening bases. As discussed hereinabove, nicking of a double-stranded nucleic acid molecule may create instability on the nicked strand. If an insufficient number of bases are linked to each other, the base pairing with the second strand may not be sufficient to keep the bases from dissociating. The cleavable or excisable moieties may be positioned such that the 5′ end of the double-stranded nucleic acid molecule may be not lost. The cleavable or excisable moieties may be positioned such that a 3′ end may be generated at the nick.

A sufficient distance that excision does not dissociate intervening bases may be at least 3, 4, 5, 6, 7, 8, 9 or 10 bases. A sufficient distance that excision does not dissociate intervening bases may be more than 3 bases. A sufficient distance that excision does not dissociate intervening bases may be at least 10 bases. A sufficient distance that excision does not dissociate intervening bases may be more than 10 bases.

A first cleavable or excisable moiety may be proximal to the 5′ end and a second cleavable or excisable moiety may be a sufficient distance from the first cleavable or excisable moiety such that excision does not dissociate intervening bases. A third cleavable or excisable moiety may be also at a sufficient distance from the first cleavable or excisable moiety such that excision does not dissociate intervening bases. The second and third cleavable or excisable moieties may be sufficiently close that excision does dissociate intervening bases.

The at least two cleavable or excisable moieties may be both sufficiently downstream from an end or an end-proximal cleavable or excisable moiety such that excision does not dissociate intervening bases. The at least two cleavable or excisable moieties may be sufficiently close to each other that excision does dissociate intervening bases. The two cleavable or excisable moieties may be proximal to each other. Cleavage of the cleavable or excisable moieties may generate a gap region rather than a nick. A “gap” may be a stretch of at least 2 missing bases. A nick may comprise a gap. A gap region may be at least 2 nucleotides in length. A gap region may be not more than 5, 6, 7, 8, 9 or 10 nucleotides in length. A gap region may be not more than 5 nucleotides in length. A sufficient distance such that excision does dissociate intervening bases may be less than 10, 9, 8, 7, 6, 5, 4, 3, 2 or 1 bases. A sufficient distance such that excision does dissociate intervening bases may be less than 10 bases. A sufficient distance such that excision does dissociate intervening bases may be not more than 3 bases.

Cleavage of these two cleavable or excisable moieties may generate a gap region in one strand. As the gap region may be not proximal to the 5′ end, there may be generated an available (e.g., “free”) 3′ end at the gap region. This end may act as a primer for an incoming polymerase enzyme that may begin elongation of a nucleic acid sequence at the gap region, thus synthesizing a new strand or portion thereof. The polymerase enzyme may use an end (e.g., 3′ end) upstream of a removed cleavable or excisable moiety as a template for strand synthesis. The polymerase enzyme may use an end (e.g., 3′ end) upstream of a nick as a template for strand synthesis. The polymerase enzyme may use an end (e.g., a 3′ end) upstream of a gap region as a template for strand synthesis. The nick may be generated by cleaving a cleavable or excisable moiety.

The double-stranded molecule may comprise at least 3 cleavable or excisable moieties, wherein a first cleavable or excisable moiety may be proximal to a 5′ capture entity. At least two of the at least 3 cleavable or excisable moieties may be at a sufficient distance from the first cleavable or excisable moiety such that excision may not induce dissociation of the intervening bases, and the at least two of the at least 3 cleavable or excisable moieties may be sufficiently close to each other such that excision may induce dissociation of the intervening bases. In such a system, a single step of contacting a cleaving agent may both free an isolated complex as well as ready a double-stranded nucleic acid molecule for sequencing.

A double-stranded nucleic acid molecule may be produced by providing a double-stranded nucleic acid molecule comprising a first strand comprising a target sequence and at least one cleavable or excisable moiety (e.g., cleavable base) 5′ to the target sequence and cleaving the at least one cleavable base. Providing a double-stranded nucleic acid molecule comprising at least one cleavable or excisable moiety (e.g., cleavable base) may comprise providing a single-stranded nucleic acid molecule comprising a sequence reverse complementary to the target sequence; providing a second nucleic acid molecule reverse-complementary to a 3′ end of the at least one single-stranded molecule, and comprising at least one cleavable or excisable moiety (e.g., cleavable base), and subjecting the second nucleic acid molecule to conditions sufficient to hybridize the at least one first single-stranded nucleic acid molecule to produce a double-stranded nucleic acid molecule.

The second nucleic acid molecule may be a second primer molecule. The second nucleic acid molecule may be an adapter. The adapter may be a double-stranded adapter. The method may further comprise extending from the second nucleic acid molecule a sequence reverse-complementary to the single-stranded nucleic acid molecule that comprises a target sequence. The method may further comprise extending from the second nucleic acid molecule a sequence reverse-complementary to the single-stranded nucleic acid molecule that comprises a target sequence. The second nucleic acid molecule may be devoid of the target sequence. The second nucleic acid molecule may be a universal primer. The second nucleic acid molecule may be reverse-complementary to a 3′ adapter region of the at least one single-stranded molecule. The 3′ region may be a reverse complement of a 5′ adapter region.

The single-stranded nucleic acid molecule may be attached to a first support. Providing the single-stranded nucleic acid molecule may comprise providing a solution comprising a plurality of first supports, each support comprising at least one first primer molecule; contacting the solution with at least one template nucleic acid molecule comprising the target sequence, and a 3′ region reverse complementary to the first primer molecule; subjecting the at least one template nucleic acid molecule to conditions sufficient to hybridize the at least one template nucleic acid molecule to the at least one first primer molecule; subjecting the at least one template nucleic acid molecule hybridized to the at least one first primer molecule to conditions sufficient to extend from the at least one first primer molecule a nucleic acid sequence reverse-complementary to the at least one template nucleic acid molecule; and subjecting the template nucleic acid molecule to conditions sufficient to dissociate the template nucleic acid molecule from the reverse-complementary molecule to produce a solution comprising at least one first support coupled to at least one first single-stranded nucleic acid molecule.

The 3′ region may be a 3′ primer adapter. The adapter may be a universal adapter and may be reverse-complementary to a support primer. The first primer molecule may be a support primer molecule. The template nucleic acid molecule may further comprise a 5′ adapter sequence. The at least one first single-stranded nucleic acid molecule may comprise a 3′ region reverse-complementary to the 5′ adapter sequence. The second nucleic acid molecule may be homologous to the 5′ adapter and thus reverse-complementary to a 3′ end of the at least one single-stranded molecule.

A plurality of second primer molecules and/or third primer molecules may also be used. A second primer molecule and/or a third primer molecule comprise at least two cleavable or excisable moieties (e.g., cleavable bases). The second primer molecule may comprise at least two cleavable or excisable moieties. The third primer molecule may comprise at least two cleavable or excisable moieties. The second nucleic acid molecule may comprise at least two cleavable or excisable moieties. At least one of the at least two cleavable or excisable moieties may be downstream of an end by a sufficient distance such that excision of the cleavable or excisable moieties does not induce dissociation of the intervening bases. One of the at least two cleavable or excisable moieties may be proximal to a 5′ capture entity of a primer molecule. At least two cleavable or excisable moieties may be downstream from a 5′ end at a sufficient distance such that excision of the cleavable or excisable moieties may not induce dissociation of the intervening bases. The at least two cleavable bases sufficiently downstream may be proximal to one another. The at least two cleavable bases sufficiently downstream may be proximal to the 3′ end of the primer molecule. By positioning the cleavable or excisable moieties at the 3′ end they will be proximal to the start of a target sequence, which may allow for a polymerase enzyme to immediately begin synthesizing the target sequence. The at least two cleavable or excisable moieties sufficiently downstream may be sufficiently close to each other such that excision may induce dissociation of intervening bases. The primer molecule may comprise at least 3 cleavable or excisable moieties, wherein a first cleavable or excisable moiety may be proximal to a 5′ capture entity, at least two of the cleavable or excisable moieties may be at a sufficient downstream distance of the first cleavable or excisable moiety such that excision may not induce dissociation of intervening bases, and sufficiently downstream at least two bases may be sufficiently close to each other such that excision may induce dissociation of intervening bases. Cleavage or excision of any one cleavable or excisable moiety of the at least three cleavable or excisable moieties may be configured to not induce dissociation of intervening bases. The dissociation or lack of dissociation may be of one or more bases (e.g., intervening bases) from the second strand of the nucleic acid molecule. Use of such a primer molecule in the enrichment methods provided herein may result in isolation of a double-stranded nucleic acid molecule such as can be used in the sequencing methods provided herein.

Non-limiting examples of possible primer sequences are provided in Table 1 below. Cleavable uracil bases are underlined. SEQ ID NO: 1 and SEQ ID NO: 2 provide primers with 5′ biotin moieties with a proximal cleavable base, so that the molecule can be freed after capture. The primers may also be used without the 5′ biotin. SEQ ID NO: 3 may be devoid of the capture entity and thus also has no 5′ proximal cleavable base. SEQ ID NO: 2 and SEQ ID NO: 3 have only a single cleavable 3′ uracil and thus only a nick may be generated upon excision. SEQ ID NO: 1 has two cleavable bases proximal to one another, such that excision may also remove the intervening bases (CAG)

| Seq ID | | 5′ |
|---|---|---|
| Number | Sequence | Biotin |
| 1 | GTUCCATCTCATCCCTGCGTGTCTCCGACUCAGU | Yes |
| 2 | GTUCCATCTCATCCCTGCGTGTCTCCGACUCAG | Yes |
| 3 | CCATCTCATCCCTOCGTGTCTCCGACUCAG | No |

Examples of cleaving agents include, but are not limited to, UDG, APE, USER. EndoVIII, RNase, EndoV, Fpg, OGG1, and light (e.g., ultraviolet light). The light may be laser light. A cleaving agent may be selected based on the cleavable or excisable moiety or moieties included in a given system such that the cleaving agent may be configured to cleave or excise the cleavable or excisable moiety or moieties. One or more different cleaving agents may be used in combination with one another. A method may further comprise contacting a nucleic acid molecule with another enzymatic agent to generate a functional nick. A functional nick may be a nick from which a polymerase enzyme can begin strand synthesis. Such an enzymatic agent may be, for example, T4 Polynucleotide kinase (T4 pnk). An enzyme configured to remove or repair a lesion (e.g., a 3′ lesion) may also be used. Examples of such repair enzyme include, but are not limed to, Apn1, Apn2 and Tpp1.

A polymerase enzyme may be a strand-displacing polymerase enzyme. The polymerase enzyme may comprise 5′ to 3′ exonuclease activity. The polymerase enzyme may be, for example, a strand-displacement (e.g., strand-displacing) polymerase enzyme and/or a polymerase enzyme with 5′ to 3′ exonuclease activity. Examples of strand displacing polymerases include, but are not limited to, Bst DNA polymerase, large fragment polymerase, and 029 polymerase. Examples of polymerases with 5′ to 3′ exonuclease activity include, but are not limited to, Taq polymerase and DNA polymerase I (non-Klenow).

A variety of polymerases can be used, many of which are commercially available. For example, a polymerase enzyme belonging to any one of families A, B and C may be used. A family A polymerase enzyme may be a single chain protein that can contain multiple enzymatic functions including polymerase, 3′ to 5′ exonuclease activity, and 5′ to 3′ exonuclease activity. A family B polymerase enzyme may have a single catalytic domain with polymerase and 3′ to 5′ exonuclease activity, as well as accessory factors. A family C polymerase enzyme may be a multi-subunit protein with polymerizing and 3′ to 5′ exonuclease activity. A family C polymerase enzyme may also have strand-displacement activity. Additional polymerases may also be used. For example, in E. coli, three types of DNA polymerases have been found, DNA polymerases I, II, and III (analogous to family A. B, and C, respectively). In eukaryotic cells, three different family B polymerases, DNA polymerases a, 8, and E are implicated in nuclear replication, and a family A polymerase, polymerase y, may be used for mitochondrial DNA replication. Other types of DNA polymerases include phage polymerases. Any such polymerase enzyme may be used in a method provided herein.

Measuring integration of a nucleotide into a nucleic acid molecule may comprise detecting the presence or absence of an incorporated labeled nucleotide, such as by detecting a signal or change in signal (e.g., fluorescent signal). The detecting may be detecting the presence or absence in an extended first strand. When the presence of an incorporated, labeled nucleotide may be detected, a presence and size or absence of a stretch of more than one base of the same type in tandem may be also detected. Detection of incorporation of multiple sequential nucleotides into a nucleic acid strand (e.g., into a homopolymeric region of a nucleic acid strand) may also be detected. The absence of a signal may be indicative of no incorporation. In some embodiments, the detecting or measuring integration of a nucleotide is sequencing of the complementary strand.

The method may further comprise washing to remove unincorporated nucleotides. The method may further comprise repeating the processes described above with a second, third, and fourth single nucleotide solution (e.g., additional nucleotide solutions comprising nucleotides including different canonical bases, including A, C, G, and T/U). These steps may be repeated until no new nucleotides are incorporated (e.g., until a nucleic acid sequence of interest is analyzed). Thus, each base may be added one at a time and incorporation may be measured, until all four bases have been added. This may be repeated until the full length of a target sequence has been sequenced. The method may comprise washing after the addition of each base, as well as, in some instances, the use of additional solutions including solutions including reagents for cleaving labels from labeled nucleotides and chase solutions including unlabeled nucleotides.

A plurality of nucleotides may be a plurality of labeled nucleotides. For example, 100% of the nucleotides in a nucleotide solution may be labeled. In another example, less than 100, 90, 80, 70, 60, 50, 40, 30, 20 or 10% of the nucleotides in a solution may be labeled. For example, fewer than 20% of the nucleotides in a nucleotide solution may be labeled. In another example, less than 100, 90, 80, 70, 60, 50, 40, 30, 20, and 10% of the nucleotides incorporated by a polymerase enzyme may be labeled nucleotides. In an example, less than 20% of the nucleotides incorporated by a polymerase enzyme may be labeled nucleotides.

Materials used in a sequencing process may to selected to optimize the sequencing process. For example, a polymerase enzyme (e.g., DNA polymerase enzyme) and labeled nucleotides that maximize incorporation efficiency and minimize incorporation variability may be used. Selection of such materials may reduce or avoid the potential for differences in the incorporation efficiency of labeled nucleotides. Various polymerases (e.g., DNA polymerases) may efficiently incorporate fluorescently-labeled nucleotides (e.g., dNTPs) along nucleic acid (e.g., DNA) templates to produce very uniform fragments independent of the sequence context. Such polymerases may be used in an automated Sanger/dideoxy-based sequencing method using a capillary-array DNA sequencer. A number of natural and engineered DNA polymerases are capable of incorporating fluorescently-labeled deoxyribonucleotides. Examples may be found in, e.g., Reeve & Fuller Nature 376, 796-97 (1995); Rosenblum et al., Nucleic Acids Res 25, 4500-04 (1997); Ramanathan et al. Anal Biochem 337, 1-11 (2005); Aksyonov et al., Anal Biochem 348, 127-138 (2006); Tabor & Richardson, J Biol Chem 265, 8322-28 (1990); Zhu et al., Nucleic Acids Res 22, 3418-22 (1994); Zhu & Waggoner Cytometry 28, 206-211 (1997); Randolph & Waggoner Nucleic Acids Res 25, 2923-29 (1997); Mitra et al., Anal Biochem 320, 55-65 (2003); Anderson et al. Biotechniques 38, 257-264 (2005), each of which is herein incorporated by reference.

A sequencing process may comprise sequencing by synthesis. Additional examples of sequencing methods are disclosed in, e.g., International Patent Applications WO2010/117804, and WO1998/044152, each of which is herein incorporated by reference in its entirety.

One or more nucleic acid molecules (e.g., double-stranded nucleic acid molecules) may be analyzed in parallel. The one or more nucleic acid molecules may be coupled to the same or different supports. The one or more nucleic acid molecules may each comprise a target sequence, which target sequence may be the same or different. For example, a plurality of double-stranded nucleic acid molecules may comprise a plurality of target sequences which may be analyzed in parallel. A plurality of double-stranded nucleic acid molecules may comprise a clonal population of nucleic acid molecules (e.g., as described herein).

Sequencing from a double-stranded nucleic acid template may reduce secondary structure dependent errors in sequence determination. A signal or change in signal produced by a nucleic acid molecule coupled to a support upon incorporation of a labeled nucleotide may be perturbed or altered based on the context of the incorporation site. Secondary structure in a target sequence can slow incorporation, reduce incorporation rate, or even repel incorporation. These types of perturbations may increase the error rate in a sequencing process even more when not all nucleotides are labeled nucleotides (e.g., when a plurality of nucleotides used in a sequencing process includes a plurality of unlabeled nucleotides). Use of double-stranded nucleic acid templates may reduce issues associated with secondary structure as the template may not anneal to itself in the same ways that a single-stranded template may.

The term “context dependence” or “context dependency,” as used herein, generally refers to signal correlations with local sequence, relative nucleotide representation, or genomic locus. Signals for a given sequence may vary due to context dependency, which may depend on the local sequence, relative nucleotide representation of the sequence, or genomic locus of the sequence.

Flow sequencing by synthesis (SBS) may comprise performing repeated nucleic acid (e.g., DNA) extension cycles, in which individual species of nucleotides and/or labeled nucleotide analogs are presented to a primer-template-polymerase complex in a so-called “flow”, which then incorporates a nucleotide if it is complementary to a next open position of a template. The product of each flow may be measured for each clonal population of templates, e.g., a bead or a colony. The resulting nucleotide incorporations may be detected and quantified by unambiguously distinguishing signals corresponding to or associated with zero, one, two, three, four, five, six, seven, eight, nine, ten, or more than ten sequential incorporations (e.g., in the case of incorporation into homopolymeric regions). Accurate quantification of such multiple sequential incorporations may comprise quantifying characteristic signals for each possible homopolymer of 0, 1, 2, . . . , N sequential nucleotides incorporated in a colony in each flow. For example, a homopolymer containing sequential A nucleotides may be represented as A. AA, AAA, . . . , up to N sequential A nucleotides. Accurate quantification of homopolymer lengths (e.g., a number of sequential identical nucleotides in a sequence) may encounter challenges owing to random and unpredictable systematic variations in signal level, which can cause errors in quantifying the homopolymer length. Accurate quantification of homopolymer lengths (e.g., a number of sequential identical nucleotides in a sequence) may also encounter challenges owing to sequence context dependent signal, which may be different for every sequence. For example, in the case of fluorescence measurements of dilute labeled nucleotides, sequence context can affect both the number of labeled analogs (variable tolerance for incorporating labeled analogs) as well as fluorescence of individual labeled analogs (e.g., quantum yield of dyes affected by local context of f5 bases, as described by (Kretschy, et al., Sequence-Dependent Fluorescence of Cy3- and Cy5-Labeled Double-Stranded DNA, Bioconjugate Chem., 27(3), pp. 840-848), which is herein incorporated by reference in its entirety). In practice, with dye-terminator Sanger cycle sequencing, substantial systematic variations in signals have been identified for 3-base contexts (e.g., as described by Zakeri, et al., “Peak height pattern in dichloro-rhodamine and energy transfer dye terminator sequencing”, Biotechniques, 25(3), pp. 406-10, which is herein incorporated by reference in its entirety).

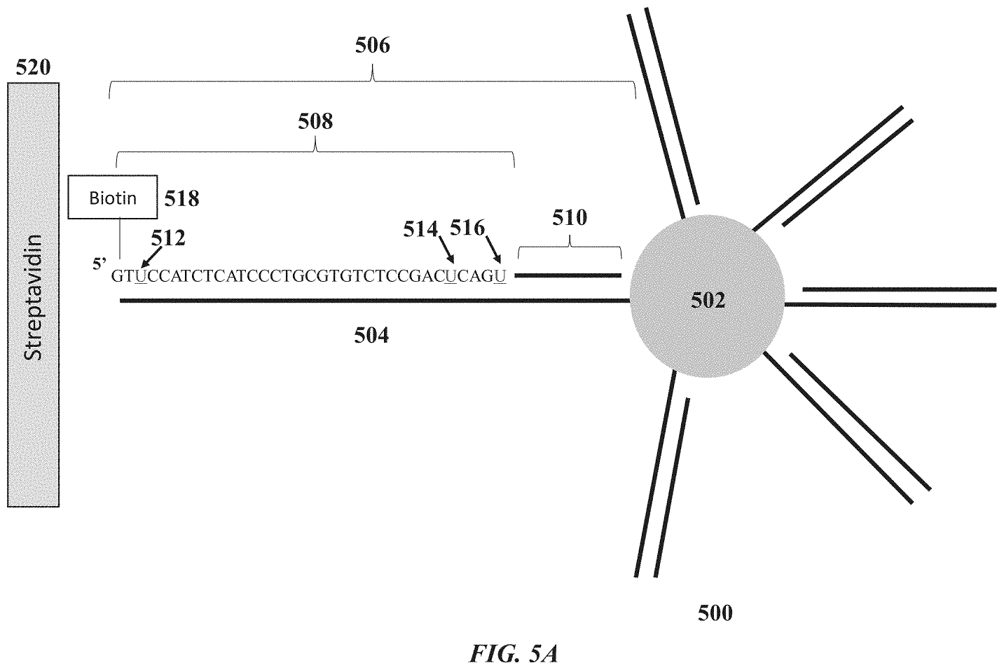

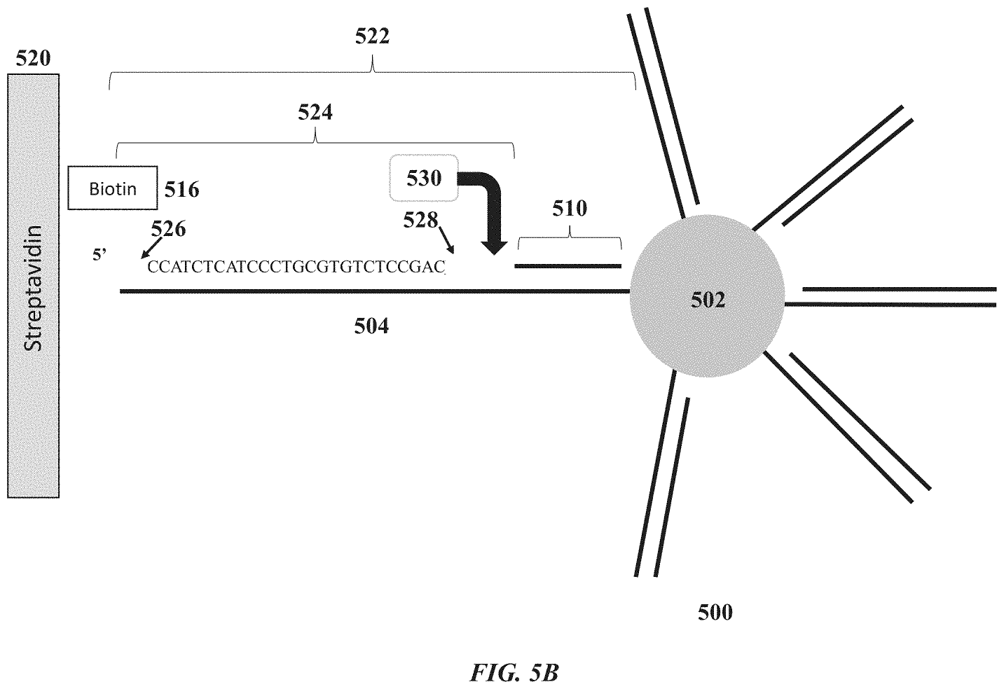

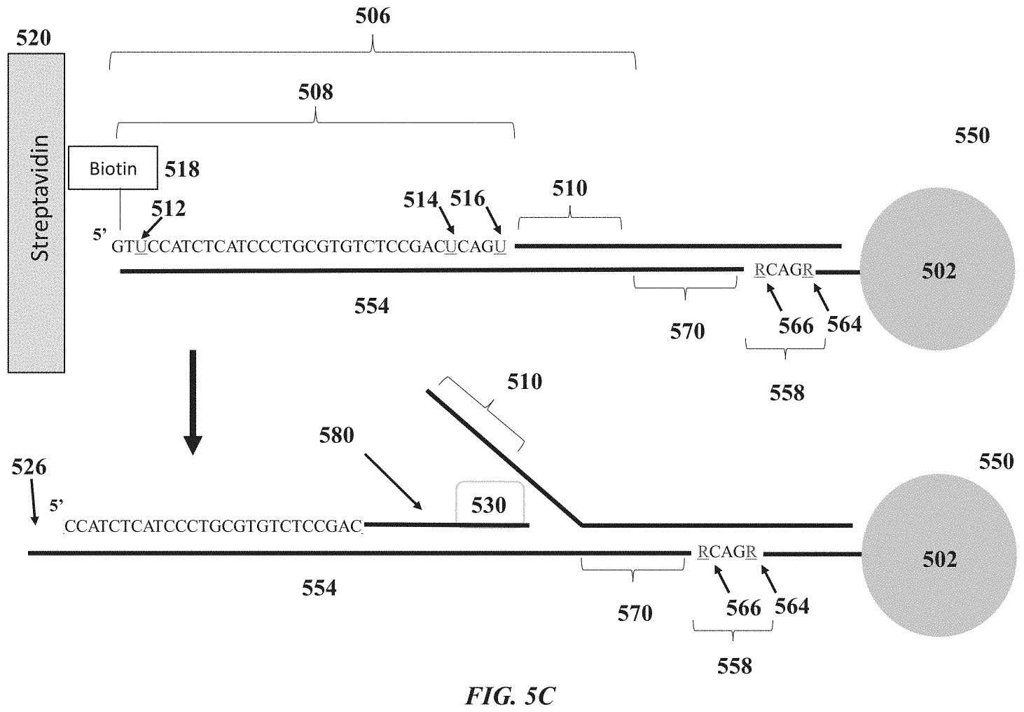

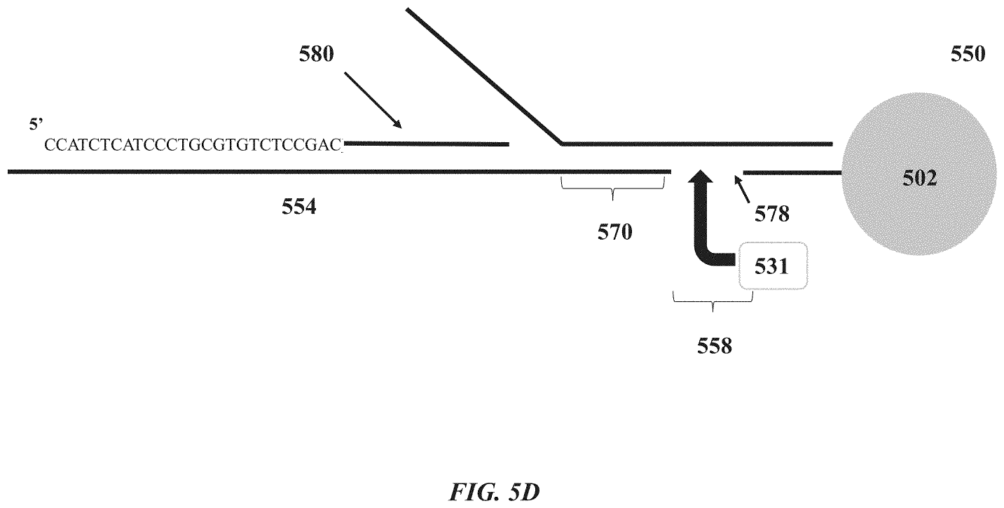

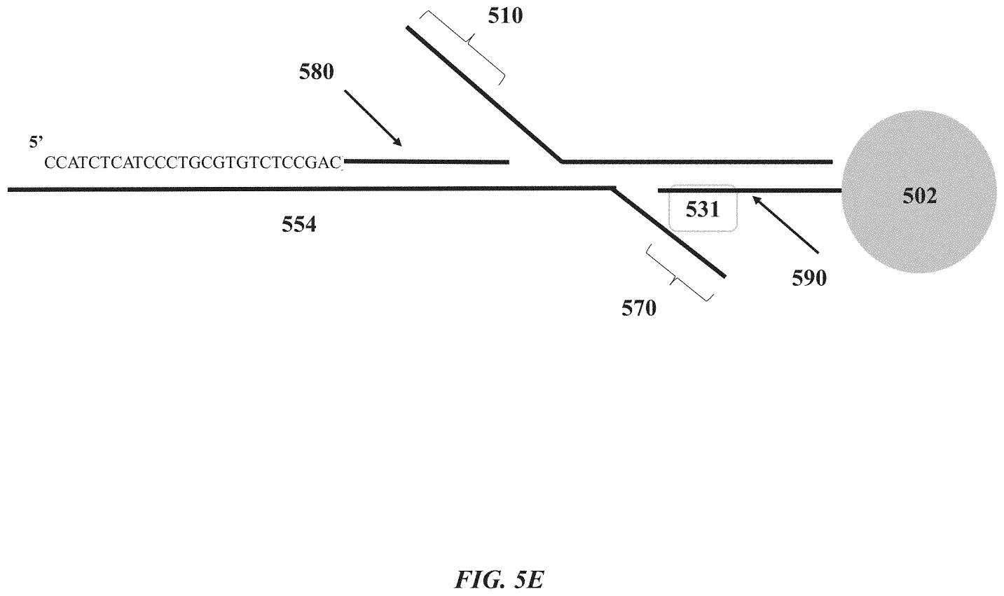

Reference is now made to FIGS. 5A-5B, which shows an exemplary sequencing method. Figure SA shows a complex 500 comprising support 502 (e.g., a bead) that has been coupled to a capturing resin 520 (e.g., streptavidin) via a 5′ linked biotin moiety 518. Many such complexes may be coupled to the resin, though for simplicity only a single complex is shown. Complex 500 comprises a plurality of nucleic acid molecules coupled thereto. One or more nucleic acid molecules of the plurality of nucleic acid molecules may include a biotin moiety and one or more cleavable or excisable moieties, though only one such example is shown. A nucleic acid molecule coupled to the support 502 comprises strand 504 and 506. Strand 506 comprises a sequence 508 including cleavable or excisable moieties 512, 514, and 516 (shown here as uracil bases). Biotin moiety 518 is coupled to a base of sequence 508 at an end of strand 506. Strand 506 also comprises a first target sequence 510. Complex 500 may be contacted with a cleaving agent such as UDG or APE (e.g., as described herein) to cleave or excise the cleavable or excisable moieties 512, 514, and 516. As shown in FIG. 5B, upon cleavage or excision of cleavable or excisable moiety 512, the additional bases at the 5′ end of strand 506 may dissociate to provide a gap region 526 at the 5′ end of sequence 524 of modified strand 522. Upon cleavage or excision of cleavable or excisable moieties 514 and 516, a gap region 528 may be generated adjacent (e.g., 5′ adjacent) to the target sequence 510. The remaining sequence 5′ to the gap region 528 may act as a primer with which polymerase enzyme 530 may interact. Polymerase enzyme 530 may begin extending from the available 3′ end of this sequence toward the target sequence 510. Sequence 510 may be dissociated from strand 504 during the extension process (e.g., if the polymerase enzyme 530 is a strand-displacement polymerase enzyme). Extension may comprise incorporation of labeled and/or unlabeled nucleotides (e.g., as described above).

Reference is now made to FIGS. 5C-5E, which show an exemplary embodiment of a pair-end sequencing method. This method is shown in combination with a pre-enrichment step, although this step is not essential. As shown in FIG. 5C, complex 550 is equivalent to complex 500 shown in FIG. 5A; the difference is that complex 550 comprises second strand 554 that comprises a sequence 558 including cleavable or excisable moieties 564 and 566 (shown here as RNA bases, “R”) which is adjacent (e.g., 5′ adjacent) to a second target sequence 570. Cleavable or excisable moieties 564 and 566 may be within the original primer conjugated to support 502 that was used to originally bind the template nucleic acid. Cleavable or excisable bases 564 and 566 in this example are cleavable by different conditions than cleavable or excisable bases 512, 514, and 516 in strand 506. Excision of cleavable or excisable bases 512, 514, and 516 is performed as described hereinabove and shown in FIG. 5B. It will be understood that cleavage of base 512 is not essential for sequencing, but it does produce gap region 526 and removes biotin moiety 518. Sequencing may be performed in the presence of the enrichment support 520 (e.g., streptavidin) or alternatively sequencing may be performed without a pre-enrichment step. Polymerase enzyme 530 begins extending from the available 3′ end of gap region 528 to produce new sequence 580 which comprises a sequence identical to first target sequence 510. When extension comprises incorporation of labeled nucleotides and incorporation of the labeled nucleotides is measured, first target sequence 510 is sequenced. Extension can be stopped by the introduction of a capped or blocked nucleotide into new sequence 580. For simplicity, only the strands being described are shown in complex 550.

Complex 550 may next be contacted with a cleaving agent such as RNase (e.g., as described herein) to cleave or excise the cleavable or excisable moieties 564, and 566. As shown in FIG. 5D, upon cleavage or excision of cleavable or excisable moieties 564 and 566, a gap region 578 may be generated adjacent (e.g., 5′ adjacent) to the second target sequence 570. The remaining sequence 5′ to the gap region 578 may act as a primer with which polymerase enzyme 531 may interact. Polymerase enzyme 531 may begin extending from the available 3′ end of this sequence toward the second target sequence 570. Polymerase enzyme 531 may be the same polymerase as polymerase enzyme 530 or it may be a different polymerase enzyme. Second target sequence 570 may be dissociated from strand 506 during the extension process (e.g., if the polymerase enzyme 530 is a strand-displacement polymerase enzyme). Extension may comprise incorporation of labeled and/or unlabeled nucleotides (e.g., as described above). As shown in FIG. 5E, polymerase enzyme 531 begins extending from the available 3′ end of gap region 528 to produce new sequence 590 which comprises a sequence identical to second target sequence 570. When extension comprises incorporation of labeled nucleotides and incorporation of the labeled nucleotides is measured, second target sequence 570 is sequenced. Extension can be stopped before generating a sequence complementary to sequence 580/510 or it can be continued to produce such a complementary sequence.

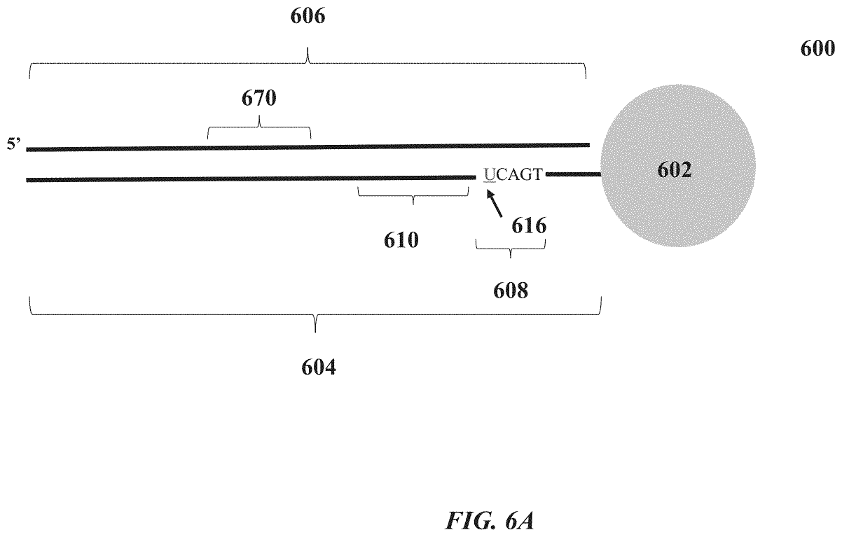

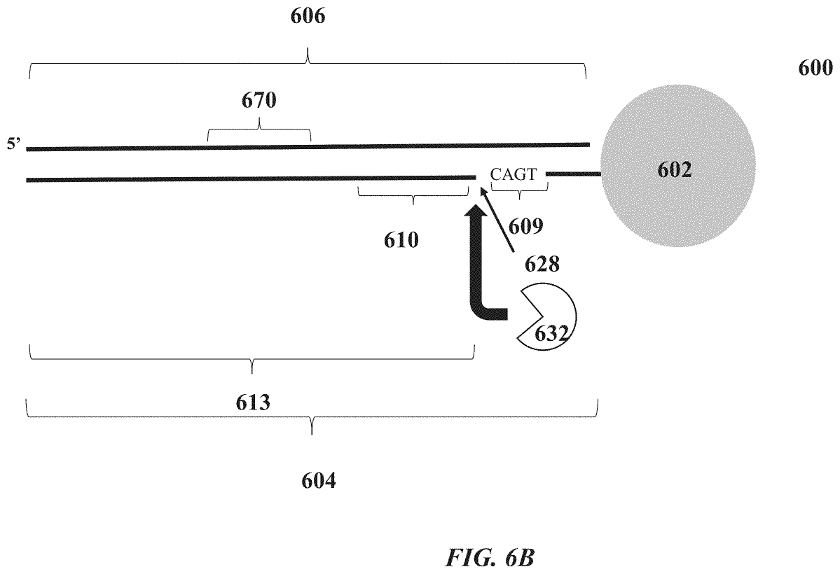

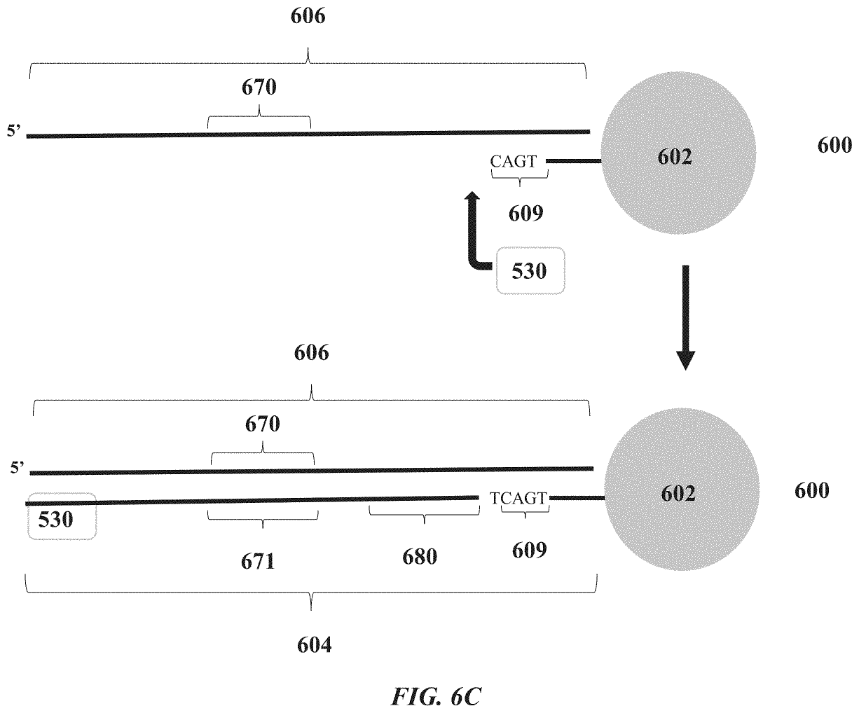

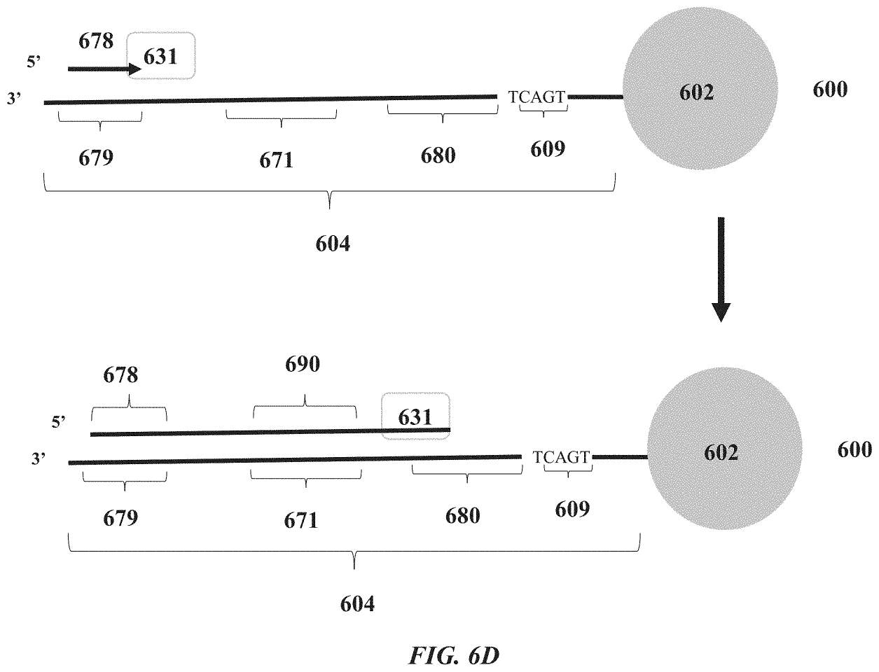

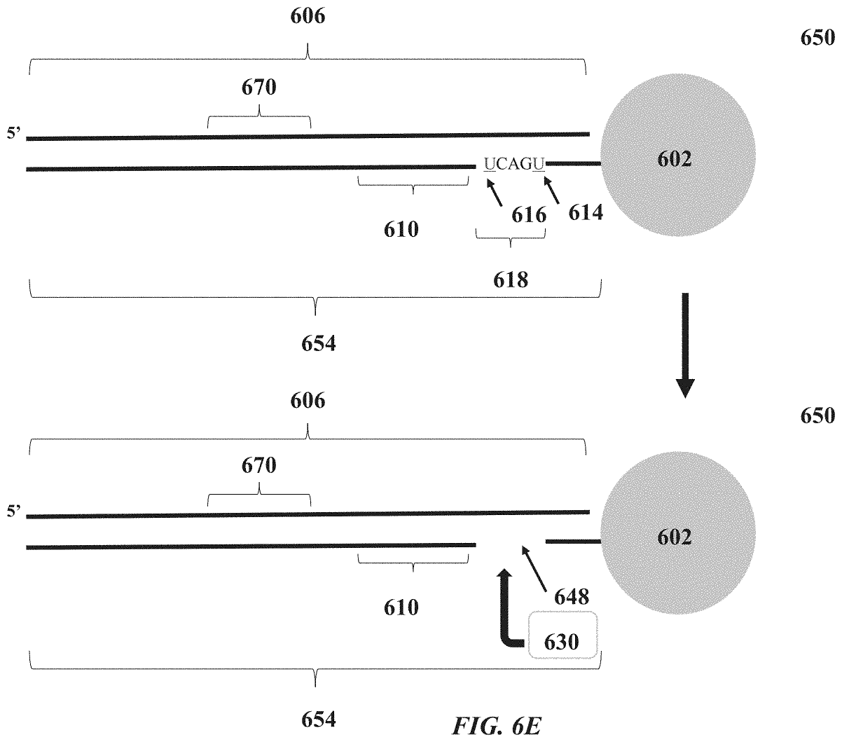

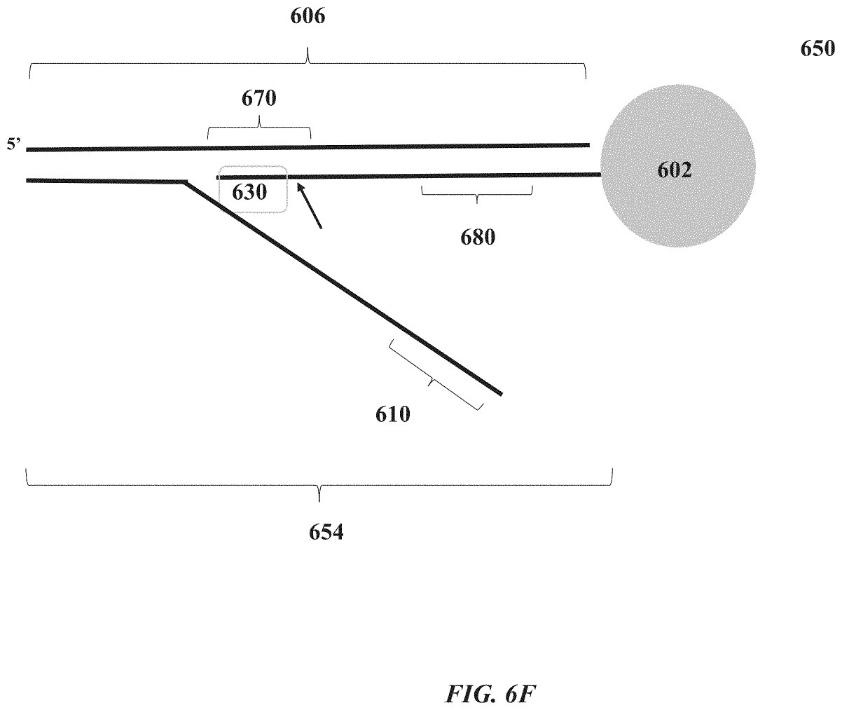

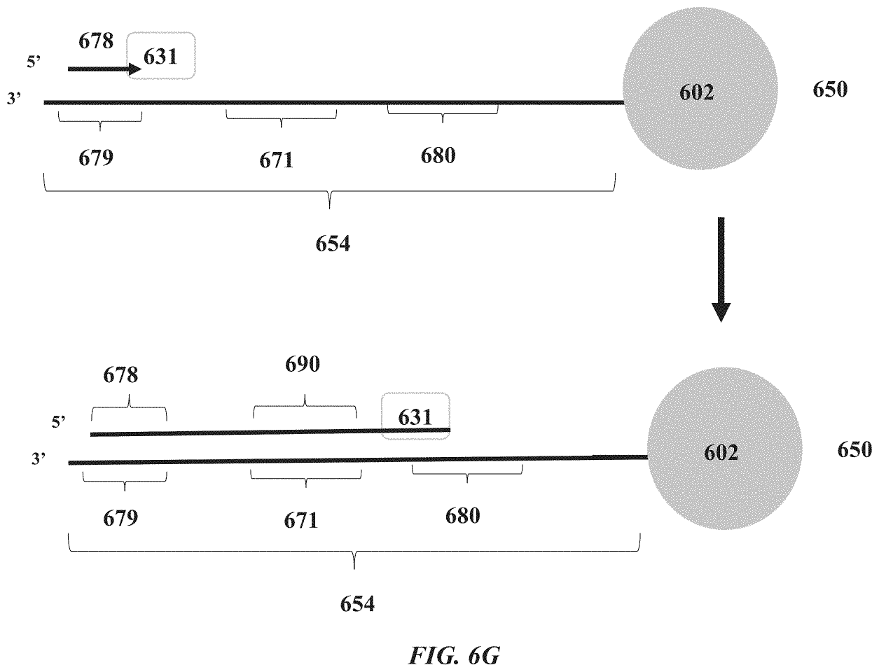

Reference is now made to FIG. 6A-6D, which show another exemplary embodiment of a pair-end sequencing method which requires only a single cleavable or excisable base within the strand conjugated to the support. This method may be combined with a method of pre-enrichment, although this is not essential. FIG. 6A shows a complex 600 comprising a plurality of nucleic acid molecules coupled thereto. One or more nucleic acid molecules of the plurality of nucleic acid molecules may include one or more cleavable or excisable moieties, though only one such example is shown. A nucleic acid molecule coupled to the support 602 comprises strand 604 and 606. Strand 604 comprises a sequence 608 including a cleavable or excisable moiety 616 (shown here as a uracil base). Strand 604 also comprises a first target sequence 610 which is adjacent (e.g., 3′ adjacent) to sequence 608. Strand 606 comprises a second target sequence 670 which is complementary to a sequence in strand 604 that is 3′ to first sequence 610. Complex 600 may be contacted with a cleaving agent such as UDG or APE (e.g., as described herein) to cleave or excise the cleavable or excisable moiety 616. In methods that include a pre-enrichment step, an additional cleavable or excisable moiety proximal to a capture entity proximal to the 5′ end of strand 606 (5′ to second target region 670) is also cleaved or excised. The capture entity is either comprised on conjugated to a base that is the additional cleavable or excisable moiety or that is 5′ to the additional cleavable or excisable moiety, such that excision of the additional cleavable or excisable moiety dissociates the capture entity from complex 600. This excision is done either by the same cleaving agent that cleaves or excises cleavable or excisable moiety 616 or by a different cleaving agent.

As shown in FIG. 6B, upon cleavage or excision of cleavable or excisable base 616, a nick 628 may be generated adjacent (e.g., 5′ adjacent) to the first target sequence 610. Sequence 609 is produced corresponding to the 5′ fragment of sequence 608 that remains after cleavage. The nick 628 contains a free 5′ end and a free 3′ end. If a plurality of cleavable or excisable moieties are employed a gap region is generated in place of a nick, 5′ to 3′ exonuclease enzyme 632 is added and digests away region 613 which is 3′ to nick 628. First target sequence 610 is degraded with digestion of region 613. Strand 606 will not be degraded as its 5′ end is protected by the presence of a phosphorothioated phosphoramidite base. As shown in FIG. 6C, sequence 609 may act as a primer with which polymerase enzyme 630 may interact. Polymerase enzyme 630 may begin extending from the available 3′ end of this sequence to produce a new sequence 680 which is identical to first target sequence 610. When extension comprises incorporation of labeled nucleotides and incorporation of the labeled nucleotides is measured, first target sequence 610 is sequenced. A new sequence 671, which is complementary to second target sequence 670, is also produced (see e.g., FIG. 63). Production of the region 3′ to new sequence 680 may be done without labeled nucleotides and may be done rapidly with a mix of nucleobases. Extension can be stopped when an entirely new strand 604 is produced or a capped or blocked nucleotide can be added to halt extension.

FIG. 6D shows sequencing of the second target sequence 670. Strand 606 is dissociated from complex 600 (e.g., by sodium hydroxide or formamide strip). Complex 600 therefore now comprises only bead (e.g., support) 602 conjugated to strand 604 which contains sequence 671 that is complementary to second target sequence 670. Sequencing primer 678, which is complementary to a sequence 679 in the 3′ end of strand 604, is added. Sequence 679 is 3′ to sequence 671. Polymerase enzyme 631 is also added. Polymerase enzyme 631 may be the same or different from polymerase enzyme 630. Primer 678 may interact with polymerase enzyme 631. Polymerase enzyme 631 may begin extending from the available 3′ end of primer 678 to produce a new sequence 690 which is identical to second target sequence 670. When extension comprises incorporation of labeled nucleotides and incorporation of the labeled nucleotides is measured, second target sequence 670 is sequenced.

Reference is now made to FIGS. 6E-6G, which show another exemplary embodiment of a pair-end sequencing method which requires a plurality of cleavable or excisable base within the strand conjugated to the support. This method may be combined with a method of pre-enrichment, although this is not essential. FIG. 6E shows a complex 650 comprising a plurality of nucleic acid molecules coupled thereto. One or more nucleic acid molecules of the plurality of nucleic acid molecules may include a plurality of cleavable or excisable moieties, though only one such example is shown. A nucleic acid molecule coupled to the support 602 comprises strand 654 and 606. Strand 654 comprises a sequence 618 including at least two cleavable or excisable moieties 614 and 616 (shown here as a uracil base). Strand 654 also comprises a first target sequence 610 which is adjacent (e.g., 3′ adjacent) to sequence 618. Strand 606 comprises a second target sequence 670 which is complementary to a sequence in strand 604 that is 3′ to first target sequence 610. Complex 650 may be contacted with a cleaving agent such as UDG or APE (e.g., as described herein) to cleave or excise the cleavable or excisable moieties 614 and 616. In methods that include a pre-enrichment step, an additional cleavable or excisable moiety proximal to a capture entity proximal to the 5′ end of strand 606 (5′ to second target region 670) is also cleaved or excised. The capture entity is either comprised on conjugated to a base that is the additional cleavable or excisable moiety or that is 5′ to the additional cleavable or excisable moiety, such that excision of the additional cleavable or excisable moiety dissociates the capture entity from complex 650. This excision is done either by the same cleaving agent that cleaves or excises cleavable or excisable moieties 614 and 616 or by a different cleaving agent. Upon cleavage or excision of cleavable or excisable bases (e.g., moieties) 614 and 616, a gap region 648 may be generated adjacent (e.g., 5′ adjacent) to the target sequence 610. The remaining sequence 5′ to the gap region 648 may act as a primer with which polymerase enzyme 630 may interact. Polymerase enzyme 630 may begin extending from the available 3′ end of this sequence toward the target sequence 610. Sequence 610 may be dissociated from strand 606 during the extension process (e.g., if the polymerase enzyme 630 is a strand-displacement polymerase enzyme). Extension may comprise incorporation of labeled and/or unlabeled nucleotides (e.g., as described above).

As can be seen in FIG. 6F, polymerase enzyme 630 may begin extending from the available 3′ end of the sequence conjugated to support 602 to produce a new sequence 680 which is identical to first target sequence 610. When extension comprises incorporation of labeled nucleotides and incorporation of the labeled nucleotides is measured, first target sequence 610 is sequenced. A new sequence 671, which is complementary to second target sequence 670, is also produced (e.g., see FIG. 6G). Production of the region 3′ to new sequence 680 may be done without labeled nucleotides and may be done rapidly with a mix of nucleobases. Extension can be stopped when an entirely new strand 654 is produced or a capped or blocked nucleotide can be added to halt extension.

FIG. 6G shows sequencing of the second target sequence 670. Strand 606 is dissociated from complex 650 (e.g., by sodium hydroxide or formamide strip). With dissociation of strand 606, the original strand displaced by polymerase enzyme 630 is also removed from complex 650. Complex 650 therefore now comprises only bead 602 conjugated to strand 654 which is essentially identical to strand 604 described hereinabove, and which contains sequence 671 that is complementary to second target sequence 670. Sequencing primer 678, which is complementary to a sequence 679 in the 3′ end of strand 654, is added. Sequence 679 is 3′ to sequence 671. Polymerase enzyme 631 is also added. Polymerase enzyme 631 may be the same or different from polymerase enzyme 630. Primer 678 may interact with polymerase enzyme 631. Polymerase enzyme 631 may begin extending from the available 3′ end of primer 678 to produce a new sequence 690 which is identical to second target sequence 670. When extension comprises incorporation of labeled nucleotides and incorporation of the labeled nucleotides is measured, second target sequence 670 is sequenced.

### Computer Systems

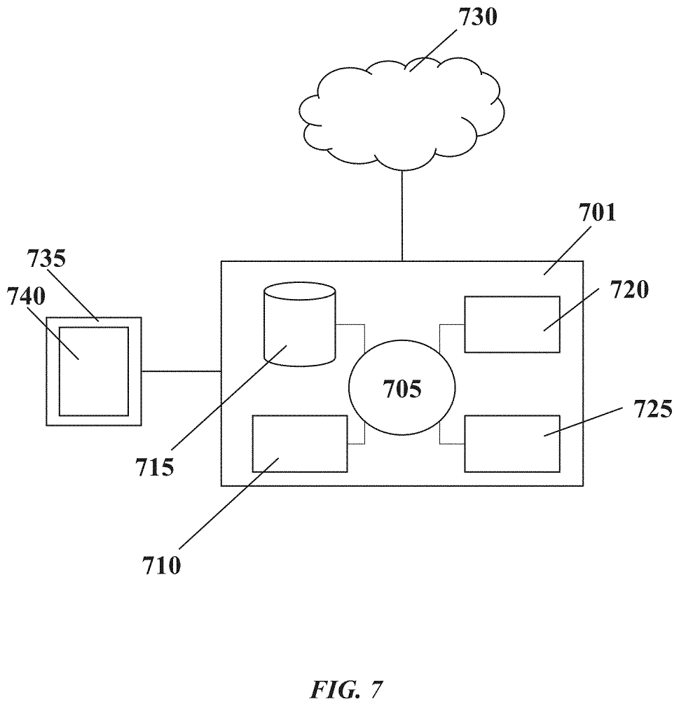

The present disclosure provides computer systems that are programmed to implement methods of the disclosure. FIG. 7 shows a computer system 701 that is programmed or otherwise configured to perform nucleic acid sequencing. The computer system 701 can determine sequence reads based at least in part on intensities of detected optical signals. The computer system 701 can regulate various aspects of the present disclosure, such as, for example, performing nucleic acid sequencing, sequence analysis, and regulating conditions of transient binding and non-transient binding (e.g., incorporation) of nucleotides. The computer system 701 can be an electronic device of a user or a computer system that is remotely located with respect to the electronic device. The electronic device can be a mobile electronic device.

The computer system 701 includes a central processing unit (CPU, also “processor” and “computer processor” herein) 705, which can be a single core or multi core processor, or a plurality of processors for parallel processing. The computer system 701 also includes memory or memory location 710 (e.g., random-access memory, read-only memory, flash memory), electronic storage unit 715 (e.g., hard disk), communication interface 720 (e.g., network adapter) for communicating with one or more other systems, and peripheral devices 725, such as cache, other memory, data storage and/or electronic display adapters. The memory 710, electronic storage unit 715, communication interface 720 and peripheral devices 725 are in communication with the CPU 705 through a communication bus (solid lines), such as a motherboard. The electronic storage unit 715 can be a data storage unit (or data repository) for storing data. The computer system 701 can be operatively coupled to a computer network (“network”) 730 with the aid of the communication interface 720. The network 730 can be the Internet, an internet and/or extranet, or an intranet and/or extranet that is in communication with the Internet. The network 730 in some cases is a telecommunication and/or data network. The network 730 can include one or more computer servers, which can enable distributed computing, such as cloud computing. The network 730, in some cases with the aid of the computer system 701, can implement a peer-to-peer network, which may enable devices coupled to the computer system 701 to behave as a client or a server.

The CPU 705 can execute a sequence of machine-readable instructions, which can be embodied in a program or software. The instructions may be stored in a memory location, such as the memory 710. The instructions can be directed to the CPU 705, which can subsequently program or otherwise configure the CPU 705 to implement methods of the present disclosure. Examples of operations performed by the CPU 705 can include fetch, decode, execute, and writeback.

The CPU 705 can be part of a circuit, such as an integrated circuit. One or more other components of the system 701 can be included in the circuit. In some cases, the circuit is an application specific integrated circuit (ASIC).

The electronic storage unit 715 can store files, such as drivers, libraries, and saved programs. The electronic storage unit 715 can store user data. e.g., user preferences and user programs. The computer system 701 in some cases can include one or more additional data storage units that are external to the computer system 701, such as located on a remote server that is in communication with the computer system 701 through an intranet or the Internet.

The computer system 701 can communicate with one or more remote computer systems through the network 730. For instance, the computer system 701 can communicate with a remote computer system of a user. Examples of remote computer systems include personal computers (e.g., portable PC), slate or tablet PC's (e.g., Apple®, iPad, Samsung® Galaxy Tab), telephones, Smart phones (e.g., Apple® iPhone. Android-enabled device, Blackberry®), or personal digital assistants. The user can access the computer system 701 via the network 730.

Methods as described herein can be implemented by way of machine (e.g., computer processor) executable code stored on an electronic storage location of the computer system 701, such as, for example, on the memory 710 or electronic storage unit 715. The machine executable or machine-readable code can be provided in the form of software. During use, the code can be executed by the processor 705. In some cases, the code can be retrieved from the electronic storage unit 715 and stored on the memory 710 for ready access by the processor 705. In some situations, the electronic storage unit 715 can be precluded, and machine-executable instructions are stored on memory 710.

The code can be pre-compiled and configured for use with a machine having a processer adapted to execute the code or can be compiled during runtime. The code can be supplied in a programming language that can be selected to enable the code to execute in a pre-compiled or as-compiled fashion.

Aspects of the systems and methods provided herein, such as the computer system 701, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 701 can include or be in communication with an electronic display 735 that comprises a user interface (UI) 740 for providing, for example, results of nucleic acid sequence and optical signal detection (e.g., sequence reads, intensity maps, etc.). Examples of UI's include, without limitation, a graphical user interface (GUI) and web-based user interface.

Methods and systems of the present disclosure can be implemented by way of one or more algorithms. An algorithm can be implemented by way of software upon execution by the central processing unit (CPU) 705. The algorithm can, for example, implement methods and systems of the present disclosure, such as determine sequence reads based at least in part on intensities of detected optical signals.

### EXAMPLES

Generally, the nomenclature used herein, and the laboratory procedures utilized in the present invention include molecular, biochemical, microbiological, and recombinant DNA techniques. Details of such techniques may be found in, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994): “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Strategies for Protein Purification and Characterization—A Laboratory Course Manual” CSHL Press (1996); all of which are incorporated by reference. Other general references are provided throughout this document.

Methods of pre-enrichment such as are described hereinabove, when performed with a single-stranded template lead to intramolecular secondary structure formation which leads to poor hybridization and enzymatic efficiency. Further, after post-enrichment beads are found to aggregate, thus making them unusable for sequencing. However, when a double-stranded template is used and released by cleavage away of the capture entity (e.g., biotin) the dropout of sequences and aggregation of beads post-enrichment are reduced. Particles with single-stranded templates and particles with double-stranded templates are pre-enriched and post-enriched and subsequently sent for sequencing. The number of clumped beads, error percentage, genome coverage, percentage of reads passing the RSQ filter, and percentage passing U-alignment are monitored. Particles with double-stranded templates are found to have less aggregation, better genome coverage and superior read quality.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1.-306. (canceled)

307. A method of processing a nucleic acid molecule, the method comprising:
a) providing a solution comprising a nucleic acid molecule coupled to a support, wherein said nucleic acid molecule comprises a first strand and a second strand, wherein (i) said first strand comprises a cleavable or excisable moiety proximal to an end of said first strand and (ii) said first strand comprises or is coupled to a capture entity at or proximal to said end;
b) bringing said solution into contact with a capturing entity under conditions sufficient to couple said capture entity and said capturing entity;
c) separating said nucleic acid molecule coupled to said capturing entity from other components of said solution; and
d) subsequent to (c), subjecting said nucleic acid molecule coupled to said capturing entity to conditions sufficient to cleave or excise said cleavable or excisable moiety, thereby uncoupling said nucleic acid molecule and said capturing entity; thereby processing the nucleic acid molecule.

308. The method of claim 307, wherein said method further comprises, prior to (a):
(i) providing said support coupled to a single-stranded nucleic acid molecule, wherein said single-stranded nucleic acid molecule comprises said second strand of said nucleic acid molecule, or a portion thereof,
(ii) providing a primer molecule comprising a nucleic acid sequence that is complementary to a nucleic acid sequence at or near an end of said single-stranded nucleic acid molecule distal to said support, wherein said primer molecule comprises said cleavable or excisable moiety and comprises or is coupled to said capture entity;
(iii) subjecting said single-stranded nucleic acid molecule and said primer molecule to conditions sufficient to hybridize said primer molecule to said single-stranded nucleic acid molecule; and
(iv) subjecting said primer molecule hybridized to said single-stranded nucleic acid molecule to conditions sufficient to extend said primer molecule to generate said first strand of said nucleic acid molecule, or a portion thereof.

309. The method of claim 308, wherein said primer molecule is coupled to a particle, and the method further comprises subjecting said primer molecule coupled to said particle to conditions sufficient to release said primer molecule from said particle.

310. The method of claim 307, wherein said method further comprises, prior to (a):
(i) providing said support coupled to a primer molecule comprising a first nucleic acid sequence;
(ii) providing a template nucleic acid molecule comprising a second nucleic acid sequence that is complementary to said first nucleic acid sequence of said primer molecule;
(iii) subjecting said template nucleic acid molecule and said primer molecule to conditions sufficient to hybridize said template nucleic acid molecule to said primer molecule; and
(iv) subjecting said primer molecule hybridized to said template nucleic acid molecule to conditions sufficient to extend said primer molecule to generate said second strand of said nucleic acid molecule, or a portion thereof.

311. The method of claim 310, further comprising (v) subjecting said second strand of said nucleic acid molecule hybridized to said template nucleic acid molecule to conditions sufficient to separate said template nucleic acid molecule and said second strand.

312. The method of claim 310, wherein said template nucleic acid molecule is coupled to a particle, and the method further comprises subjecting said template nucleic acid molecule coupled to said particle to conditions sufficient to release said template nucleic acid molecule from said particle.

313. The method of claim 311, further comprising (vi) providing an additional primer molecule comprising (A) a third nucleic acid sequence that is complementary to a fourth nucleic acid sequence of said second strand and (B) a cleavable or excisable moiety, or non-cleavable or excisable analog thereof, wherein said additional primer molecule comprises or is coupled to said capture entity; (vii) subjecting said second strand and said additional primer molecule to conditions sufficient to hybridize said additional primer molecule to said second strand; and (viii) subjecting said additional primer molecule hybridized to said second strand to conditions sufficient to extend said additional primer molecule to generate said first strand of said nucleic acid molecule, or a portion thereof.

314. The method of claim 313, wherein said additional primer molecule is coupled to a particle, and the method further comprises subjecting said additional primer molecule coupled to said particle to conditions sufficient to release said additional primer molecule from said particle.

315. The method of claim 307, wherein said solution comprises a plurality of supports coupled to a plurality of nucleic acid molecules comprising a plurality of capture entities and a plurality of cleavable or excisable moieties, wherein each nucleic acid molecule of said plurality of nucleic acid molecules comprises a respective first strand and a respective second strand, wherein (i) each said respective first strand comprises a respective cleavable or excisable moiety of said plurality of cleavable or excisable moieties proximal to a respective free end of each said respective first strand and (ii) each said respective first strand comprises or is coupled to a respective capture entity of said plurality of capture entities at or proximal to said respective free end.

316. The method of claim 315, wherein said solution further comprises a plurality of additional supports, which plurality of additional supports is not coupled to nucleic acid molecules comprising capture entities.

317. The method of claim 307, wherein said cleavable or excisable moiety is selected from the group consisting of a ribonucleic acid (RNA) base, a uracil base, an inosine base, 2,6-diamino-4-hydroxy-5-formamidopyrimidine (FapyG) base, 8-oxo-7,8- dihydroguanine (8oxoG) base, and a photocleavable base.

318. The method of claim 317, wherein said nucleic acid molecule comprises deoxyribonucleic acid (DNA) and said cleavable or excisable moiety is an RNA base, and wherein said nucleic acid molecule is devoid of RNA bases other than said cleavable or excisable moiety.

319. The method of claim 307, wherein said conditions in (d) comprise bringing said nucleic acid molecule in contact with a cleaving agent configured to cleave or excise said cleavable or excisable moiety.

320. The method of claim 319, wherein said cleaving agent is one or more members selected from the group consisting of uracil DNA glycosylase (UDG), apyrimidinic/apurinic endonuclease (APE), endonuclease, endonuclease VIII (EndoVIII), endonuclease V (EndoV), uracil-specific excision reagent (USER) enzyme, formamidopyrimidine DNA glycosylase (Fpg), 8-oxoguanine glycosylase (OGGI), RNase, RNaseH, RNaseHII, and ultraviolet light.

321. The method of claim 307, wherein said capture entity comprises one or more members selected from the group consisting of biotin, avidin, a nucleic acid sequence, a charged particle, and a magnetic particle.

322. The method of claim 321, wherein said capture entity comprises biotin.

323. The method of claim 321, wherein said capture entity comprises a nucleic acid sequence.

324. The method of claim 307, wherein said capturing entity comprises one or more members selected from the group consisting of biotin, avidin, a nucleic acid sequence, an electric field system, and a magnetic field system.

325. The method of claim 307, wherein said capturing entity is coupled to another support.

326. The method of claim 307, wherein said nucleic acid molecule comprises an additional cleavable or excisable moiety.


SEQUENCE LISTING (3 sequences)

SEQ ID NO: 1 - DNA, 34 bp, synthetic construct
  misc_feature 1..34: note = Synthetic
  source 1..34: mol_type = other DNA
  modified_base 3: mod_base = OTHER; note = uracil
  modified_base 30: mod_base = OTHER; note = uracil
  modified_base 34: mod_base = OTHER; note = uracil
gttccatctcatccctgcgtgtctccgactcagt

SEQ ID NO: 2 - DNA, 33 bp, synthetic construct
  misc_feature 1..33: note = Synthetic
  source 1..33: mol_type = other DNA
  modified_base 3: mod_base = OTHER; note = uracil
  modified_base 30: mod_base = OTHER; note = uracil
gttccatctcatccctgcgtgtctccgactcag

SEQ ID NO: 3 - DNA, 30 bp, synthetic construct
  misc_feature 1..30: note = Synthetic
  source 1..30: mol_type = other DNA
  modified_base 27: mod_base = OTHER; note = uracil
ccatctcatccctgcgtgtctccgactcag